(19) United States
(12) Patent Application Publication
(10) Pub. No.: US 2024/0336951 A1
(43) Pub. Date: Oct. 10, 2024

(54) METHODS, COMPOSITIONS, AND KITS FOR CHARACTERIZING SKINS

(71) Applicant: Mitra Bio Limited, LONDON (GB)

(72) Inventors: Cristiana Banila, LONDON (GB); Shakiba Kaveh, LONDON (GB)

(21) Appl. No.: 18/290,994

(22) PCT Filed: Sep. 7, 2022
(86) PCT No.: PCT/GB2022/052271

§ 371 Date: Jan. 22, 2024

(30) Foreign Application Priority Data
Sep. 8, 2021 (GB) 2112817.8

(51) Int. Cl. C12Q 1/6806; A61B 10/02; C12Q 1/6874

(52) U.S. Cl. CPC C12Q 1/6806; A61B 10/02; C12Q 1/6874

(57) ABSTRACT

Provided herein are methods for determining epidermal properties of a subject. The methods may comprise assaying nucleic acids in an epidermal sample of the subject. The methods also comprise identifying the genome, epigenome, and/or metagenome in the epidermal sample of the subject. Also provided are the compositions and kits for carrying out the same.

### CROSS-REFERENCE

This application claims priority to United Kingdom Patent Application No. GB2112817.8, filed Sep. 8, 2021, which is entirely incorporated herein by reference.

### BACKGROUND

Proper skin care can protect the skin, the outermost organ exposed to foreign elements. A properly maintained skin not only provides health benefits but also aesthetic interests. However, proper skin care demands proper characterization of a skin condition, which may entail a large number of epidermal samples. Obtaining such samples may require professional handling of the samples or invasive sampling methods. Currently available methodologies are also limited due to the lack of robustness and sensitivity. Current methodologies may not allow population-wide determination of epidermal properties due to a lack of facilities or professionals that can carry out the task. Invasive sampling methods may also prohibit proper characterization or determination of epidermal properties across a wide population.

### SUMMARY

Provided herein are methods for identifying epidermal properties. Kits, systems, or reagents for doing the same are also provided. The methods and reagents provided herein can allow self-sampling of an epidermal sample that does not require a professional. The methods and reagents provided herein can also allow non-invasive sampling of the epidermal sample. A subject can extract the epidermal sample for epidermal property determination by oneself without assistance of a professional. Using the methods and reagents described herein can obtain a sufficient amount of nucleic acids from the epidermal sample for downstream analysis to determine the epidermal property of the subject. The determination of the epidermal property can facilitate therapeutic regimen designs to improve the epidermal property of the subject. The methods and reagents provided herein can also allow for population-wide determination of epidermal properties.

Provided herein, are methods for determining an epidermal property of a subject, In an aspect, a method for determining an epidermal property of a subject, comprising: (a) providing an epidermal sample of the subject that has been obtained non-invasively from the subject; (b) processing at least a portion of nucleic acids derived from the epidermal sample of the subject using at least an enzyme which is capable of modifying a base of a nucleotide of the at least the portion of the nucleic acids thereby producing modified nucleic acids; and (c) determining the epidermal property of the subject based at least partially on the modified nucleic acids.

In some embodiments, the modifying in (b) comprises converting the base into a different base. In some embodiments, the enzyme comprises an enzyme of a Ten-Eleven Translocation (TET) family, an enzyme of a uridine diphosphate (UDP)-glucose:deoxyribonucleic acid (DNA) beta-D-glucosyltransferase family, or an enzyme of a deaminase family. In some embodiments, the enzyme comprises the enzyme of the TET family. In some embodiments, the enzyme of the TET family comprises TET1, TET2, TET3, or a functional equivalent derivative thereof. In some embodiments, the enzyme of the TET family comprises the TET2 or a functional equivalent derivative thereof. In some embodiments, the enzyme comprises the enzyme of the UDP-glucose:DNA beta-D-glucosyltransferase family. In some embodiments, the enzyme of the UDP-glucose:DNA beta-D-glucosyltransferase family is a UDP-glucose:DNA beta-D-glucosyltransferase from E. coli. In some embodiments, the enzyme comprises the enzyme of the deaminase family. In some embodiments, the enzyme of the deaminase family comprises Apolipoprotein B mRNA Editing Catalytic Polypeptide-like 1 (APOBEC1), Apolipoprotein B mRNA Editing Catalytic Polypeptide-like 3A (APOBEC3A), Apolipoprotein B mRNA Editing Catalytic Polypeptide-like 3B (APOBEC3B), Apolipoprotein B mRNA Editing Catalytic Polypeptide-like 3C (APOBEC3C), Apolipoprotein B mRNA Editing Catalytic Polypeptide-like 3D (APOBEC3D), Apolipoprotein B mRNA Editing Catalytic Polypeptide-like 3E (APOBEC3E), Apolipoprotein B mRNA Editing Catalytic Polypeptide-like 3F (APOBEC3F), Apolipoprotein B mRNA Editing Catalytic Polypeptide-like 3H (APOBEC3H), Apolipoprotein B mRNA Editing Catalytic Polypeptide-like 3G (APOBEC3G), Activation-induced cytidine deaminase (AICDA), Cytidine deaminase (CDA), deoxycytidylate monophosphate (dCMP) deaminase (DCTD), or a functional equivalent derivative thereof. In some embodiments, the enzyme of the deaminase family comprises the APOBEC3A or a functional equivalent derivative thereof. In some embodiments, (b) comprises processing the at least the portion of nucleic acids derived from the epidermal sample of the subject using TET2 or a functional equivalent derivative thereof, UDP-glucose:DNA beta-D-glucosyltransferase from E. coli or a functional equivalent derivative thereof, and APOBEC3A or a functional equivalent derivative thereof.

In some embodiments, the method further comprises assaying at least a portion of the modified nucleic acids to identify a genome and an epigenome of the epidermal sample, and wherein (c) further comprises determining the epidermal property of the subject based at least partially on the genome and the epigenome of the epidermal sample. In some embodiments, the epigenome comprises a methylome. In some embodiments, the method further comprises obtaining the epidermal sample non-invasively from the subject. In some embodiments, obtaining the epidermal sample non-invasively from the subject comprises applying an adhesive article to a skin area of the subject. In some embodiments, the adhesive article comprises a skin patch. In some embodiments, the epidermal sample comprises cells from stratum corneum of the subject. In some embodiments, the cells comprise primary cells. In some embodiments, the primary cells comprise skin cells. In some embodiments, the skin cells comprise keratinocytes. In some embodiments, the keratinocytes comprise corneocytes.

In some embodiments, the method further comprises determining a methylation level of at least a portion of the modified nucleic acids, and wherein (c) further comprises determining the epidermal property of the subject based at least partially on the methylation level. In some embodiments, the method further comprises determining an epidermal property of a reference subject at least in part by determining a reference methylation level of at least a portion of nucleic acids derived from an epidermal sample of the reference subject, and wherein (c) further comprises determining the epidermal property of the subject based at least partially on the reference methylation level. In some embodiments, the method further comprises comparing the methylation level with the reference methylation level, and wherein (c) further comprises determining the epidermal property of the subject based at least partially on the comparing. In some embodiments, the method further comprises determining a hypermethylation or hypomethylation of the methylation level relative to the reference methylation level, and wherein (c) further comprises determining the epidermal property of the subject based at least partially on the hypermethylation or hypomethylation of the methylation level. In some embodiments, a difference between the methylation level and the reference methylation level is indicative of a difference in the epidermal property of the subject and the epidermal property of the reference subject. In some embodiments, the method further comprises, prior to (b), deriving the nucleic acids from the epidermal sample. In some embodiments, the deriving comprises extracting the nucleic acids from the epidermal sample. In some embodiments, deriving the nucleic acids from the epidermal sample further comprises treating at least a portion of the extracted nucleic acids. In some embodiments, the treating comprises performing an amplification reaction, a reverse transcription reaction, a primer extension reaction, a ligation reaction, or a combination thereof. In some embodiments, the nucleic acids derived from the epidermal sample comprise deoxyribonucleic acid (DNA), ribonucleic acid (RNA), or a combination or variant thereof. In some embodiments, the nucleic acids derived from the epidermal sample comprise coding sequences or non-coding sequences. In some embodiments, the nucleic acids derived from the epidermal sample comprise the coding sequences. In some embodiments, the nucleic acids derived from the epidermal sample comprise the non-coding sequences. In some embodiments, the nucleic acids derived from the epidermal sample comprise the coding sequences and the non-coding sequences. In some embodiments, the method further comprises sequencing at least a portion of the modified nucleic acids to produce sequencing reads, and wherein (c) further comprises determining the epidermal property of the subject based at least partially on the sequencing reads. In some embodiments, the sequencing comprises whole-genome sequencing. In some embodiments, the sequencing comprises whole-genome methylation sequencing. In some embodiments, the sequencing comprises next-generation sequencing. In some embodiments, the sequencing comprises chain termination sequencing, high-throughput sequencing, mass spectrophotometry sequencing, massively parallel signature sequencing, Maxam-Gilbert sequencing, nanopore sequencing, primer walking, pyrosequencing, Sanger sequencing, semiconductor sequencing, sequencing-by-hybridization, sequencing-by-ligation, sequencing-by-synthesis, single-molecule sequencing, or shotgun sequencing. In some embodiments, the sequencing comprises sequencing-by-synthesis.

In some embodiments, the epidermal property comprises intrinsic epidermal age, extrinsic epidermal age, epidermal firmness and elasticity, moisture, regeneration capacity, antioxidant protection capacity, inflammatory control, extent of epidermal infection, extent of epidermal disease condition, extent of epidermal pigmentation, extent of ultraviolet (UV) exposure, extent of UV damage, or a combination thereof. In some embodiments, the epidermal property comprises a presence or an absence of a skin disease, disorder, or condition. In some embodiments, the skin disease, disorder, or condition is selected from the group consisting of a bruise, a sebaceous cyst, a skin tag, actinic keratoses, allergic contact dermatitis, allergic purpura, an allergic reaction, an infection, basal cell carcinoma, dermatitis, Dermatitis herpetiformis, erythema multiforme, erythema nodosum, hidradenitis suppurative, hives, irritant contact dermatitis, Kaposi's sarcoma, keloid, lipoma, lupus erythematosus, melanoma, Pityriasis rosea, psoriasis, seborrhea, squamous cell carcinoma, xanthelasma, or a combination thereof. In some embodiments, the method further comprises (d) administering a therapeutic regimen to the subject based on the presence of the skin disease, disorder, or condition. In some embodiments, the therapeutic regimen alleviates or improves the epidermal property. In some embodiments, the therapeutic regimen comprises an inorganic compound, an organic compound, a drug, a peptide, a protein, a nucleic acid, or a combination thereof. In some embodiments, the administering comprises topical application of the therapeutic regimen. In some embodiments, the topical application comprises a hydration mask. In some embodiments, the administering comprises an injection of the therapeutic regimen. In some embodiments, the injection comprises onabotulinumtoxinA (Botox®), deoxycholic acid, calcium hydroxylapatite, hyaluronic acid, poly-1-lactic acid, polymethylmethacrylate, bovine collagen filler, or a combination thereof. In some embodiments, the administering comprises application of a chemical peel. In some embodiments, chemical peel comprises a chemical solution that causes skin exfoliation and peeling. In some embodiments, the therapeutic regimen comprises surgery. In some embodiments, the surgery comprises cryosurgery, laser surgery, dermabrasion, liposuction, microlipoinjection, microneedling, micropigmentation, microwave thermolysis, neck lift, laser rejuvenation, body contouring, skin tightening, vein treatment, or a combination thereof. In some embodiments, the method further comprises providing a second epidermal sample from a skin area of the subject from which the epidermal sample has been obtained. In some embodiments, the method further comprises performing one or more additional iterations of (b) and (c) to monitor the epidermal property of the subject responsive to the administering.

Provided herein, are methods for determining an epidermal property of a subject, In an aspect, a method for determining an epidermal property of a subject, comprising: (a) providing an epidermal sample of the subject that has been obtained non-invasively from the subject; (b) sequencing at least a portion of nucleic acids derived from the epidermal sample of the subject to determine a transcriptome and a genome of the epidermal sample; and (c) determining the epidermal property of the subject based at least partially on the transcriptome and the genome of the epidermal sample.

In some embodiments, the genome comprises an epigenome. In some embodiments, the epigenome comprises a methylome. In some embodiments, the method further comprises obtaining the epidermal sample non-invasively from the subject. In some embodiments, obtaining the epidermal sample non-invasively from the subject comprises applying an adhesive article to a skin area of the subject. In some embodiments, the adhesive article comprises a skin patch. In some embodiments, (b) further comprises determining a metagenome of the epidermal sample, and wherein (c) further comprises determining the epidermal property of the subject based at least partially on the metagenome of the epidermal sample. In some embodiments, the metagenome comprises a bacterium, a virus, an archaeon, a fungus, a protozoon, or a combination thereof. In some embodiments, the metagenome comprises at least two of the bacterium, the virus, the archaea, the fungus, and the protozoon. In some embodiments, the metagenome comprises at least three of the bacterium, the virus, the archaea, the fungus, or the protozoon. In some embodiments, the metagenome comprises the bacterium, the virus, the archaea, the fungus, and the protozoon. In some embodiments, the epidermal sample comprises cells from stratum corneum of the subject. In some embodiments, the cells comprise primary cells. In some embodiments, the primary cells comprise skin cells. In some embodiments, the skin cells comprise keratinocytes. In some embodiments, the keratinocytes comprise corneocytes.

In some embodiments, the method further comprises, prior to (b), deriving the nucleic acids from the epidermal sample. In some embodiments, the deriving further comprises extracting the nucleic acids from the epidermal sample. In some embodiments, the deriving further comprises treating at least a portion of the extracted nucleic acids. In some embodiments, the treating comprises performing an amplification reaction, a reverse transcription reaction, a primer extension reaction, a ligation reaction, or a combination thereof. In some embodiments, the extracted nucleic acids or the nucleic acids derived from the epidermal sample comprise deoxyribonucleic acid (DNA), ribonucleic acid (RNA), or a combination or variant thereof. In some embodiments, the extracted nucleic acids or the nucleic acids derived from the epidermal sample comprise coding sequences or non-coding sequences. In some embodiments, the extracted nucleic acids or the nucleic acids derived from the epidermal sample comprise the coding sequences. In some embodiments, the extracted nucleic acids or the nucleic acids derived from the epidermal sample comprise the non-coding sequences. In some embodiments, the extracted nucleic acids or the nucleic acids derived from the epidermal sample comprise the coding sequences and the non-coding sequences. In some embodiments, the sequencing comprises whole-genome sequencing. In some embodiments, the sequencing comprises whole-genome methylation sequencing. In some embodiments, the sequencing comprises next-generation sequencing. In some embodiments, the sequencing comprises chain termination sequencing, high-throughput sequencing, mass spectrophotometry sequencing, massively parallel signature sequencing, Maxam-Gilbert sequencing, nanopore sequencing, primer walking, pyrosequencing, Sanger sequencing, semiconductor sequencing, sequencing-by-hybridization, sequencing-by-ligation, sequencing-by-synthesis, single-molecule sequencing, or shotgun sequencing. In some embodiments, the sequencing comprises sequencing-by-synthesis.

In some embodiments, the epidermal property comprises intrinsic epidermal age, extrinsic epidermal age, epidermal firmness and elasticity, moisture, regeneration capacity, antioxidant protection capacity, inflammatory control, extent of epidermal infection, extent of epidermal disease condition, extent of epidermal pigmentation, extent of ultraviolet (UV) exposure, extent of UV damage, or a combination thereof. In some embodiments, the epidermal property comprises a presence or an absence of a skin disease, disorder, or condition. In some embodiments, the skin disease, disorder, or condition is selected from the group consisting of a bruise, a sebaceous cyst, a skin tag, actinic keratoses, allergic contact dermatitis, allergic purpura, an allergic reaction, an infection, basal cell carcinoma, dermatitis, Dermatitis herpetiformis, erythema multiforme, erythema nodosum, hidradenitis suppurative, hives, irritant contact dermatitis, Kaposi's sarcoma, keloid, lipoma, lupus erythematosus, melanoma, Pityriasis rosea, psoriasis, seborrhea, squamous cell carcinoma, xanthelasma, or a combination thereof. In some embodiments, the method further comprises (d) administering a therapeutic regimen to the subject based on the presence of the skin disease, disorder, or condition. In some embodiments, the therapeutic regimen alleviates or improves the epidermal property. In some embodiments, the therapeutic regimen comprises an inorganic compound, an organic compound, a drug, a peptide, a protein, a nucleic acid, or a combination thereof. In some embodiments, the administering comprises topical application of the therapeutic regimen. In some embodiments, the topical application comprises a hydration mask. In some embodiments, the administering comprises an injection of the therapeutic regimen. In some embodiments, the injection comprises onabotulinumtoxinA (Botox®), deoxycholic acid, calcium hydroxylapatite, hyaluronic acid, poly-1-lactic acid, polymethylmethacrylate, bovine collagen filler, or a combination thereof. In some embodiments, the administering comprises application of a chemical peel. In some embodiments, chemical peel comprises a chemical solution that causes skin exfoliation and peeling. In some embodiments, the therapeutic regimen comprises surgery. In some embodiments, the surgery comprises cryosurgery, laser surgery, dermabrasion, liposuction, microlipoinjection, microneedling, micropigmentation, microwave thermolysis, neck lift, laser rejuvenation, body contouring, skin tightening, vein treatment, or a combination thereof. In some embodiments, the method further comprises providing a second epidermal sample from a skin area of the subject from which the epidermal sample has been obtained. In some embodiments, the method further comprises performing one or more additional iterations of (b) and (c) to monitor the epidermal property of the subject responsive to the administering.

Provided herein, are methods for determining an epidermal property of a subject, In an aspect, a method for determining an epidermal property of a subject, comprising: (a) providing an epidermal sample of the subject; (b) assaying nucleic acid molecules derived from the epidermal sample to identify: (i) a first set of nucleic acid sequences from the subject; and (ii) a second set of nucleic acid sequences from a source different from the subject; @ using a trained algorithm to process the first set of nucleic acid sequences and the second set of nucleic acid sequences to determine a likelihood of the epidermal sample being positive or negative for the epidermal property.

In some embodiments, the source different from the subject comprises a species different from the subject. In some embodiments, the species comprises a bacterium, a virus, an archaeon, a fungus, a protozoon, or a combination thereof. In some embodiments, the epidermal sample is obtained in an invasive, non-invasive, or semi-invasive manner. In some embodiments, the epidermal sample is obtained in a non-invasive manner. In some embodiments, the non-invasive manner comprises applying an adhesive article to a skin area of the subject. In some embodiments, the adhesive article comprises a skin patch. In some embodiments, the epidermal sample is obtained in an invasive manner. In some embodiments, the invasive manner comprises a biopsy. In some embodiments, the biopsy comprises a punch biopsy.

In some embodiments, the first set of nucleic acid sequences comprises a transcriptome, a genome, or a combination thereof. In some embodiments, the first set of nucleic acid sequences comprises the genome. In some embodiments, the genome comprises an epigenome. In some embodiments, the epigenome comprises a methylome. In some embodiments, the second set of nucleic acid sequences of the nucleic acids comprises a metagenome. In some embodiments, the epidermal sample comprises cells from stratum corneum of the subject. In some embodiments, the cells comprise primary cells. In some embodiments, the primary cells comprise skin cells. In some embodiments, the skin cells comprise keratinocytes. In some embodiments, the keratinocytes comprise corneocytes. In some embodiments, the method further comprises, prior to (b), extracting the nucleic acid molecules from the epidermal sample. In some embodiments, the method further comprises, prior to (b), processing the nucleic acid molecules extracted from the epidermal sample to form the nucleic acid molecules derived from the epidermal sample. In some embodiments, the processing comprises an amplification reaction, a reverse transcription reaction, or a combination thereof. In some embodiments, the processing comprises the amplification reaction. In some embodiments, the processing comprises the reverse transcription reaction. In some embodiments, the nucleic acid molecules derived from the epidermal sample comprise deoxyribonucleic acid (DNA), ribonucleic acid (RNA), or a combination or variant thereof. In some embodiments, the nucleic acid molecules derived from the epidermal sample comprise DNA. In some embodiments, the nucleic acid molecules derived from the epidermal sample comprise RNA. In some embodiments, the nucleic acid molecules derived from the epidermal sample comprise DNA and RNA. In some embodiments, the first set of nucleic acid sequences comprises coding sequences and/or non-coding sequences. In some embodiments, the first set of nucleic acid sequences comprises the coding sequences. In some embodiments, the first set of nucleic acid sequences comprises the non-coding sequences. In some embodiments, the first set of nucleic acid sequences comprises the coding sequences and the non-coding sequences. In some embodiments, the assaying of (b) comprises sequencing. In some embodiments, the assaying of (b) comprises whole-genome sequencing. In some embodiments, the assaying of (b) comprises whole-genome methylation sequencing. In some embodiments, the assaying of (b) comprises next-generation sequencing. In some embodiments, the assaying of (b) comprises chain termination sequencing, high-throughput sequencing, mass spectrophotometry sequencing, massively parallel signature sequencing, Maxam-Gilbert sequencing, nanopore sequencing, primer walking, pyrosequencing, Sanger sequencing, semiconductor sequencing, sequencing-by-hybridization, sequencing-by-ligation, sequencing-by-synthesis, single-molecule sequencing, or shotgun sequencing. In some embodiments, the assaying of (b) comprises sequencing-by-synthesis.

In some embodiments, the epidermal property comprises intrinsic epidermal age, extrinsic epidermal age, epidermal firmness and elasticity, moisture, regeneration capacity, antioxidant protection capacity, inflammatory control, extent of epidermal infection, extent of epidermal disease condition, extent of epidermal pigmentation, extent of UV exposure, extent of UV damage, or a combination thereof. In some embodiments, (c) further comprises determining a likelihood of occurrence or non-occurrence of the epidermal property in the subject using the trained algorithm. In some embodiments, the trained algorithm comprises supervised learning, non-supervised learning, semi-supervised learning or any combination thereof. In some embodiments, the method further comprises (d) administering a therapeutic regimen to the subject. In some embodiments, the therapeutic regimen alleviates or improves the epidermal property. In some embodiments, the method further comprises providing an additional epidermal sample of the subject. In some embodiments, the additional epidermal sample is obtained from a skin area of the subject from which the epidermal sample is obtained. In some embodiments, the method further comprises repeating steps (b)-(c) one or more times to determine the epidermal property of the subject subsequent to the subject being administered to the therapeutic regimen.

Provided herein, are methods for determining an epidermal property of a subject, In an aspect, a method for determining an epidermal property of a subject, comprising: (a) obtaining an epidermal sample non-invasively from the subject; (b) processing at least a portion of nucleic acids derived from the epidermal sample; and (c) determining the epidermal property of the subject based at least partially on the portion of the nucleic acids processed in (b), wherein $ the portion of the nucleic acids comprises a sequence of a genome of the subject defined in Table 11 or a functional equivalent thereof.

In some embodiments, (b) comprises processing the portion of nucleic acids derived from the epidermal sample using at least an enzyme which is capable of modifying a base of a nucleotide of the portion of the nucleic acids In some embodiments, the modifying the base comprises converting the base into a different base. In some embodiments, the enzyme comprises an enzyme of a Ten-Eleven Translocation (TET) family, an enzyme of a UDP-glucose:DNA beta-D-glucosyltransferase family, or an enzyme of a deaminase family. In some embodiments, the enzyme of the TET family comprises TET1, TET2, TET3, or a functional equivalent derivative thereof. In some embodiments, the enzyme of the TET family comprises the TET2 or a functional equivalent derivative thereof. In some embodiments, the enzyme of the UDP-glucose:DNA beta-D-glucosyltransferase family is a UDP-glucose:DNA beta-D-glucosyltransferase from E. coli. In some embodiments, the enzyme of the deaminase family comprises Apolipoprotein B mRNA Editing Catalytic Polypeptide-like 1 (APOBEC1), Apolipoprotein B mRNA Editing Catalytic Polypeptide-like 3A (APOBEC3A), Apolipoprotein B mRNA Editing Catalytic Polypeptide-like 3B (APOBEC3B), Apolipoprotein B mRNA Editing Catalytic Polypeptide-like 3C (APOBEC3C), Apolipoprotein B mRNA Editing Catalytic Polypeptide-like 3D (APOBEC3D), Apolipoprotein B mRNA Editing Catalytic Polypeptide-like 3E (APOBEC3E), Apolipoprotein B mRNA Editing Catalytic Polypeptide-like 3F (APOBEC3F), Apolipoprotein B mRNA Editing Catalytic Polypeptide-like 3H (APOBEC3H), Apolipoprotein B mRNA Editing Catalytic Polypeptide-like 3G (APOBEC3G), Activation-induced cytidine deaminase (AICDA), Cytidine deaminase (CDA), deoxycytidylate monophosphate (dCMP) deaminase (DCTD), or a functional equivalent derivative thereof. In some embodiments, the enzyme of the deaminase family comprises the APOBEC3A or a functional equivalent derivative thereof. In some embodiments, (b) comprises processing at least the portion of nucleic acids derived from the epidermal sample of the subject using the TET2 or any functional equivalent derivative thereof, the UDP-glucose:DNA beta-D-glucosyltransferase from E. coli or any functional equivalent derivative thereof, and the APOBEC3A or any functional equivalent derivative thereof. In some embodiments, the method further comprises, prior to (c), assaying the at least the portion of the nucleic acids to identify a genome and an epigenome of the epidermal sample. In some embodiments, the epigenome comprises a methylome.

In some embodiments, obtaining the epidermal sample non-invasively from the subject comprises applying an adhesive article to a skin area of the subject. In some embodiments, the adhesive article comprises a skin patch. In some embodiments, the epidermal sample comprises cells from stratum corneum of the subject. In some embodiments, (c) comprises determining a methylation level of the portion of the nucleic acid processed in (b). In some embodiments, the method further comprises determining an epidermal property of a reference subject, wherein In some embodiments, the method further comprises comparing the methylation level of the portion of the nucleic acid derived from the epidermal sample of the subject with the methylation level of the portion of the nucleic acid derived from the epidermal sample of the reference subject. In some embodiments, a difference in the methylation level of the portion of the nucleic acid derived from the epidermal sample of the subject and the methylation level of the portion of the nucleic acid derived from the epidermal sample of the reference subject represents a difference in the epidermal property of the subject and the epidermal property of the reference subject.

In some embodiments, the method further comprises, prior to (b), extracting the nucleic acids from the epidermal sample. In some embodiments, the method further comprises, prior to (b), treating the nucleic acids extracted from the epidermal sample to form the nucleic acids derived from the epidermal sample. In some embodiments, the treating comprises an amplification reaction, a reverse transcription reaction, or a combination thereof. In some embodiments, the nucleic acids derived from the epidermal sample comprise deoxyribonucleic acid (DNA), ribonucleic acid (RNA), or a combination or variant thereof. In some embodiments, the nucleic acids derived from the epidermal sample comprise coding sequences or non-coding sequences. In some embodiments, the nucleic acids derived from the epidermal sample comprise coding sequences. In some embodiments, the nucleic acids derived from the epidermal sample comprise non-coding sequences. In some embodiments, the nucleic acids derived from the epidermal sample comprise coding sequences and non-coding sequences. In some embodiments, the method further comprises, prior to (c), sequencing the nucleic acids derived from the epidermal sample. In some embodiments, the sequencing comprises whole-genome sequencing. In some embodiments, the sequencing comprises whole-genome methylation sequencing. In some embodiments, the sequencing comprises next-generation sequencing. In some embodiments, the sequencing comprises chain termination sequencing, high-throughput sequencing, mass spectrophotometry sequencing, massively parallel signature sequencing, Maxam-Gilbert sequencing, nanopore sequencing, primer walking, pyrosequencing, Sanger sequencing, semiconductor sequencing, sequencing-by-hybridization, sequencing-by-ligation, sequencing-by-synthesis, single-molecule sequencing, or shotgun sequencing. In some embodiments, the sequencing comprises sequencing-by-synthesis.

In some embodiments, the epidermal property comprises intrinsic epidermal age, extrinsic epidermal age, epidermal firmness and elasticity, moisture, regeneration capacity, antioxidant protection capacity, inflammatory control, extent of epidermal infection, extent of epidermal disease condition, extent of epidermal pigmentation, extent of UV exposure, extent of UV damage, or a combination thereof. In some embodiments, the method further comprises (d) administering a therapeutic regimen to the subject. In some embodiments, the therapeutic regimen alleviates or improves the epidermal property. In some embodiments, the method further comprises obtaining an additional epidermal sample from a skin area of the subject from which the epidermal sample is obtained. In some embodiments, the method further comprises repeating steps (b)-(c) one or more times to determine the epidermal property of the subject subsequent to the subject being administered to the therapeutic regimen.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (“FIG.” and “FIGs.” herein), of which:

FIG. 1 depicts a schematic flow chart for identifying an epidermal property of a subject.

FIG. 2 depicts a schematic flow chart for processing an epidermal sample of a subject and determining an epidermal property of the epidermal sample.

FIG. 3 depicts a schematic flow chart for designing a therapeutic regimen to improve or modify an epidermal property of a subject.

FIGS. 4A-4F show the total DNA yield and quality control (QC) of the DNA extraction method described herein. FIG. 4A shows a comparison of the total DNA yield using the DNA extraction method described herein and other methods. FIG. 4B shows a comparison of the total DNA yield extracted by using magnetic beads and silica column.

FIG. 4C shows the reproducibility of the total DNA yield extracted in different replicates from different subjects. FIG. 4D shows a comparison of the total DNA yield extracted when the epidermal sample is collected by a professional nurse and via a subject's self-sampling.

FIG. 4E shows the reproducibility of the total DNA yield extracted in different replicates from different body parts. FIG. 4F shows a QC analysis of fragmented DNAs extracted using the DNA extraction method described herein.

FIG. 5 shows the proportion of methylated DNA (Y-axis) versus the distance relative to the transcription start site (TSS; X-axis).

FIG. 6 shows a comparison of DNA methylation patterns obtained using bisulfite conversion derived from biopsy samples (BS), enzymatic conversion of the DNA (EMS) derived from biopsy samples, and enzymatic conversion of the DNA (nEMS) derived from skin samples obtained non-invasively.

FIG. 7 shows that the proportion of methylation in the nucleic acid (Y-axis) from the sample obtained by skin patch is comparable to those obtained by biopsy.

FIGS. 8A-8B show a comparison of the DNA methylation landscapes between biopsy and non-invasive sampling from the same individual. FIG. 8A shows a correlation plot between the levels of different methylation sites identified from the biopsy and non-invasive sampling. FIG. 8B shows the levels of methylation between the 10 samples obtained from five individuals either via invasive and non-invasive sampling.

FIG. 9 shows the reproducibility of DNA methylation patterns collected with non-invasive sampling from different body parts.

FIGS. 10A-10B show that whole-genome sequencing of epidermal sample obtained by skin patches identifies human and non-human sequences. FIG. 10A shows the proportion of sequence reads mappable and unmappable to the human genome from the epidermal sample identified by individual skin patches. FIG. 10B shows the proportion (the percentage; Y-axis) of the total sequence reads of FIG. 10A and the theoretical proportion for human genome with various percentage of GC content (X-axis). The portion of sequence reads with elevated GC content, relative to the theoretical proportion and highlighted by an oval, suggests that these reads originated in the microbiome in the sample.

FIG. 11 shows the collection of both human and microbial DNAs with non-invasive sampling.

FIG. 12 shows methylation differences between UV-exposed and UV-protected epidermal samples.

FIGS. 13A-13B show that DNA methylation patterns observed from non-invasive sampling change with UV exposure. FIG. 13A shows a principal component analysis showing separation between UV-protected and UV-exposed samples based on methylation differences. FIG. 13B shows a heatmap showing separation between UV-protected and UV-exposed samples based on methylation differences. 2 and −2 in the gradient scale depict most hypermethylated and most hypomethylated sites, respectively.

FIGS. 14A-14B show that DNA methylation patterns observed from non-invasive sampling change with age. FIG. 14A shows a principal component analysis showing variations in age based on methylation differences. FIG. 14B shows a heatmap showing variations in age based on methylation differences. 2 and −2 in the gradient scale depict most hypermethylated and most hypomethylated sites, respectively.

FIG. 15 shows methylation differences between untreated UV-exposed epidermal samples and the comparable UV-exposed epidermal samples treated with various skin products.

FIG. 16 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “deaminase” as used herein refers to a class of enzyme that catalyzes the removal of an amino group from a nucleotide of a nucleic acid.

The term “epigenetic” as used herein refers to the gene expression that is regulated, controlled, influenced, affected, or effected by non-genetic materials.

The term “epigenome” as used herein refers to a complete set or a substantial portion of a complete set of genes or genetic materials expressed that are epigenetically regulated.

The term “epidermis” or “epidermal” as used herein refers to the outermost layer of the skin.

The term “functional equivalent derivative,” as used herein when referring to a protein or polypeptide, refers to a chemical entity that can complement, compensate, or substitute the protein or polypeptide in a functional test.

The term “genome” as used herein refers to a complete set or a substantial portion of a complete set of genes or genetic material present in a cell or an organism.

The term “invasive” as used herein when referring to epidermal sampling refers to the process of extracting a sample from a subject that requires an introduction of an instrument into the body of the subject or results in the exposure of an internal body fluid or cavity.

The term “metagenome” as used herein refers to a complete set or a substantial portion of a complete set of microorganisms present in an environment.

The term “methylated DNA” as used herein refers to the nucleotide(s) in the DNA that is/are methylated. In some cases, a methylated nucleotide in a genome may be referred to as a “methylation mark” or “methylated mark.”

The term “methylome” as used herein refers to a complete set or a substantial portion of a complete set of methylated nucleic acid present in a cell or an organism. Methylome may comprise a complete set or a substantial portion of a complete set of methylated DNA present in a cell or an organism.

The term “nucleic acid” as used herein refers to a polymer of nucleotides. Unless otherwise specified, a nucleic acid may refer to a nucleic acid molecule or a portion thereof, a nucleic acid sequence or a portion thereof, a nucleic acid fragment, or a nucleic acid species.

The term “nucleic acid extraction” as used herein refers to a process to isolate and purify a nucleic acid from a cell, cell material, or organism.

The term “primary cell” as used herein refers to a cell that is isolated from its parental tissue in its parental organism.

The term “sequencing” as used herein refers to the process of determining the primary structure or the order of the arrangement of the nucleotide of a nucleic acid. The primary structure of a nucleic acid determined by sequencing may refer to a sequence or a sequence read.

The term “skin” as used herein refers to the outermost organ of an organism. In human, skin may have three layers: epidermis, dermis, and hypodermis.

The term “transcriptome” as used herein refers to a complete set or a substantial portion of a complete set of protein-coding sequences or transcripts present in a cell or an organism.

Whenever the term “at least,” “greater than,” or “greater than or equal to” precedes the first numerical value in a series of two or more numerical values, the term “at least” or “greater than” applies to each one of the numerical values in that series of numerical values.

Whenever the term “no more than,” “less than,” or “less than or equal to” precedes the first numerical value in a series of two or more numerical values, the term “no more than” or “less than” applies to each one of the numerical values in that series of numerical values.

As used herein, the singular forms “a,” “an,” and “the” include plural references unless the context clearly dictates otherwise.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, 5%, or 1%, per the practice in the art. Alternatively, particularly with respect to skin systems or processes, the term can mean within an order of magnitude, or up to 2-fold, of a value. Where particular values can be described in the application and claims, unless otherwise stated, the term “about” meaning up to an acceptable error range for the particular value should be assumed. Also, where ranges, subranges, or both, of values can be provided, the ranges or subranges can include the endpoints of the ranges or subranges. The terms “substantially,” “substantially no,” “substantially free,” and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### Methods for Identifying Skin Properties

### Methods

Provided herein are methods for determining an epidermal property of an epidermal sample. The subject may be a human subject or a non-human subject. The subject may be having or suspected of having an epidermal condition. In some cases, the subject may be healthy. In some cases, the subject may be suffering or at risk of suffering from a disease, comprising a skin disease.

The epidermal sample may be obtained from the subject invasively, or non-invasively. In some cases, one or more nucleic acids may be extracted from the epidermal sample or a portion thereof. In some cases, a method may comprise: (a) providing an epidermal sample of the subject that has been obtained non-invasively from the subject; (b) sequencing nucleic acids derived from the epidermal sample of the subject to identify a transcriptome and a genome of the epidermal sample; and (c) determining the epidermal property of the subject based at least partially on the transcriptome and the genome of the epidermal sample. In some cases, a method for determining an epidermal property of a subject may comprise: (a) providing an epidermal sample of the subject that has been obtained non-invasively from the subject; (b) sequencing nucleic acids derived from the epidermal sample of the subject to identify a transcriptome, a genome, and a metagenome of the epidermal sample; and (c) determining the epidermal property of the subject based at least partially on the transcriptome and the genome of the epidermal sample.

In some instances, the method may comprise: (a) providing an epidermal sample of the subject that has been obtained non-invasively from the subject; (b) extracting nucleic acids from the epidermal sample; (c) sequencing nucleic acids derived from the epidermal sample of the subject to identify a transcriptome and a genome of the epidermal sample; and (d) determining the epidermal property of the subject based at least partially on the transcriptome and the genome of the epidermal sample. In some cases, a method for determining an epidermal property of a subject may comprise: (a) providing an epidermal sample of the subject that has been obtained non-invasively from the subject; (b) extracting nucleic acids from the epidermal sample; (c) processing the nucleic acids extracted from the epidermal sample to form the nucleic acids derived from the epidermal sample; (d) sequencing nucleic acids derived from the epidermal sample of the subject to identify a transcriptome and a genome of the epidermal sample; and (e) determining the epidermal property of the subject based at least partially on the transcriptome and the genome of the epidermal sample.

In some instances, the method may comprise: (a) providing an epidermal sample of the subject; (b) assaying nucleic acid molecules derived from the epidermal sample to identify: (i) a first set of nucleic acid sequences from the subject; and (ii) a second set of nucleic acid sequences from a source different from the subject; (c) using a trained algorithm to process the first set of nucleic acid sequences and the second set of nucleic acid sequences to determine a likelihood of the epidermal sample being positive or negative for the epidermal property.

In some instances, the method may comprise: (a) providing an epidermal sample of the subject; (b) extracting nucleic acid molecules from the epidermal sample; (c) assaying nucleic acid molecules derived from the epidermal sample to identify: (i) a first set of nucleic acid sequences from the subject; and (ii) a second set of nucleic acid sequences from a source different from the subject; (d) using a trained algorithm to process the first set of nucleic acid sequences and the second set of nucleic acid sequences to determine a likelihood of the epidermal sample being positive or negative for the epidermal property. In some cases, a method for determining an epidermal property of a subject may comprise: (a) providing an epidermal sample of the subject; (b) extracting nucleic acid molecules from the epidermal sample; (c) processing the nucleic acid molecules extracted from the epidermal sample to form the nucleic acid molecules derived from the epidermal sample; (d) assaying nucleic acid molecules derived from the epidermal sample to identify: (i) a first set of nucleic acid sequences from the subject; and (ii) a second set of nucleic acid sequences from a source different from the subject; (e) using a trained algorithm to process the first set of nucleic acid sequences and the second set of nucleic acid sequences to determine a likelihood of the epidermal sample being positive or negative for the epidermal property. In some cases, a method may comprise using a trained algorithm to process the first set of nucleic acid sequences and the second set of nucleic acid sequences to determine a likelihood of occurrence or non-occurrence of the epidermal property in the subject. In some cases, the likelihood may comprise a probability.

In some instances, the method may comprise: (a) providing an epidermal sample of the subject that has been obtained non-invasively from the subject; (b) processing at least a portion of nucleic acids derived from the epidermal sample of the subject using at least an enzyme which is capable of modifying a base of a nucleotide of the portion of the nucleic acids; and (c) determining the epidermal property of the subject based at least partially on the portion of the nucleic acids. In some cases, a method for determining an epidermal property of a subject may comprise: (a) providing an epidermal sample of the subject that has been obtained non-invasively from the subject; (b) processing at least a portion of nucleic acids derived from the epidermal sample of the subject using at least an enzyme which is capable of modifying a base of a nucleotide of the portion of the nucleic acids; (c) assaying at least a portion of the nucleic acids to identify a genome and an epigenome of the epidermal sample; and (d) determining the epidermal property of the subject based at least partially on the portion of the nucleic acids.

In some instances, the method may comprise: (a) providing an epidermal sample of the subject that has been obtained non-invasively from the subject; (b) extracting nucleic acids from the epidermal sample; (c) processing at least a portion of nucleic acids derived from the epidermal sample of the subject using at least an enzyme which is capable of modifying a base of a nucleotide of the portion of the nucleic acids; and (d) determining the epidermal property of the subject based at least partially on the portion of the nucleic acids. In some instances, a method for determining an epidermal property of a subject may comprise: (a) providing an epidermal sample of the subject that has been obtained non-invasively from the subject; (b) extracting nucleic acids from the epidermal sample; (c) treating the nucleic acids extracted from the epidermal sample to form the nucleic acids derived from the epidermal sample; (d) processing at least a portion of nucleic acids derived from the epidermal sample of the subject using at least an enzyme which is capable of modifying a base of a nucleotide of the portion of the nucleic acids; and (e) determining the epidermal property of the subject based at least partially on the portion of the nucleic acids. In some instances, the method comprise sequencing the nucleic acids derived from the epidermal sample prior to determining the epidermal property of the subject based at least partially on the portion of the nucleic acids.

In some instances, any methods thereof may comprise administering a therapeutic regimen to the subject. In some cases, the therapeutic regimen may alleviate or improve the epidermal property. In some cases, the therapeutic regimen may alleviate the epidermal property. In some cases, the therapeutic regimen may improve the epidermal property.

In some instances, the methods thereof may also comprise obtaining an additional epidermal sample. In some cases, the methods thereof may also comprise obtaining an additional epidermal sample from a skin area of the subject from which the epidermal sample is obtained. In some cases, the skin area from which the additional epidermal sample is obtained may be the same as the area from the which the epidermal sample is obtained. In other cases, the skin area from which the additional epidermal sample is obtained may be different from the area from the which the epidermal sample is obtained.

In some instances, the methods thereof may also comprise repeating the steps of: (1) sequencing nucleic acids derived from the epidermal sample of the subject to identify a transcriptome and a genome and/or a metagenome of the epidermal sample and (2) determining the epidermal property of the subject based at least partially on the transcriptome and the genome of the epidermal sample one or more times to determine the epidermal property of the subject subsequent to the subject being administered to the therapeutic regimen. In some cases, the methods comprise performing additional iterations of step (1) and (2) to monitor the epidermal property of the subject. In some cases, the methods comprise performing additional iterations of step (1) and (2) to monitor the epidermal property of the subject subsequent responsive to the administering subject.

In some cases, the methods thereof may also comprise repeating the steps of: (1) assaying nucleic acid molecules derived from the epidermal sample to identify: (i) a first set of nucleic acid sequences from the subject; and (ii) a second set of nucleic acid sequences from a source different from the subject; (2) using a trained algorithm to process the first set of nucleic acid sequences and the second set of nucleic acid sequences to determine a likelihood of the epidermal sample being positive or negative for the epidermal property one or more times to determine the epidermal property of the subject subsequent to the subject being administered to the therapeutic regimen. In some cases, the methods comprise performing additional iterations of step (1) and (2) to monitor the epidermal property of the subject. In some cases, the methods comprise performing additional iterations of step (1) and (2) to monitor the epidermal property of the subject subsequent responsive to the administering subject.

In some cases, the methods thereof may also comprise repeating the steps of: (1) processing at least a portion of nucleic acids derived from the epidermal sample of the subject using at least an enzyme which is capable of modifying a base of a nucleotide of the portion of the nucleic acids; and (2) determining the epidermal property of the subject based at least partially on the portion of the nucleic acids one or more times to determine the epidermal property of the subject subsequent to the subject being administered to the therapeutic regimen. In some cases, the methods comprise performing additional iterations of step (1) and (2) to monitor the epidermal property of the subject. In some cases, the methods comprise performing additional iterations of step (1) and (2) to monitor the epidermal property of the subject subsequent responsive to the administering subject.

### Epidermal Sample

In some instances, an epidermal may comprise a skin sample. In some cases, an epidermal sample may comprise a primary cell from the skin. In some cases, an epidermal sample may comprise skin cells. In some cases, an epidermal sample may comprise cells from stratum basale, stratum spinosum, stratum granulosum, stratum lucidum, stratum corneum, or a combination thereof. In some cases, an epidermal sample may comprise cells from at least two of stratum basale, stratum spinosum, stratum granulosum, stratum lucidum, or stratum corneum. In some cases, an epidermal sample may comprise cells from at least three of stratum basale, stratum spinosum, stratum granulosum, stratum lucidum, or stratum corneum. In some cases, an epidermal sample may comprise cells from at least four of stratum basale, stratum spinosum, stratum granulosum, stratum lucidum, or stratum corneum.

Epidermis may comprise multiple layers: stratum basale, stratum spinosum, stratum granulosum, stratum lucidum, and stratum corneum. Epidermal cells may comprise melanocytes or keratinocytes. Epidermis may also comprise cells from various microorganisms.

In some instances, an epidermal sample may comprise a keratinocyte, a melanocyte, a Langerhans cell, a Merkel cell, or a combination thereof. In some instances, an epidermal sample may comprise at least two of a keratinocyte, a melanocyte, a Langerhans cell, or a Merkel cell. In some instances, an epidermal sample may comprise at least three of a keratinocyte, a melanocyte, a Langerhans cell, or a Merkel cell. In some cases, an epidermal sample may comprise a keratinocyte. In some cases, an epidermal sample may comprise a melanocyte. In some cases, an epidermal sample may comprise a Langerhans cell. In some cases, an epidermal sample may comprise a Merkel cell. In some cases, a keratinocyte may be differentiated. In some cases, a keratinocyte may be undifferentiated. In some cases, a differentiated keratinocyte may comprise a terminal differentiated keratinocyte. In other cases, a keratinocyte may comprise a corneocyte.

In some cases, when comparing the epidermal properties of two epidermal samples, the two epidermal samples may comprise epidermal samples from two different subjects. The two different subjects may comprise a subject and a reference subject. In some cases, a subject or a reference subject may comprise a mammal. A subject or a reference subject may comprise a human. A reference subject may have a different epidermal property of a subject. A reference subject may have a same epidermal property of a subject. When obtaining the two epidermal samples, the two epidermal samples may be derived from the same body part. In other cases, the two epidermal samples may be derived from different body parts.

In some cases, when comparing the epidermal properties of two epidermal samples, the two epidermal samples may comprise epidermal samples from two different body parts of the same subject. The two different epidermal samples may comprise an epidermal sample and a reference epidermal sample. The two different epidermal samples may have different epidermal properties. The two different epidermal samples may have a same epidermal property.

In some cases, when comparing the epidermal properties of two epidermal samples, the two epidermal samples may comprise an epidermal sample that has been treated with a therapeutic regimen and one that has not. The two different epidermal samples may have different epidermal properties. The two different epidermal samples may have a same epidermal property.

### Epidermal Sampling

In some instances, an epidermal sample may be obtained invasively, semi-invasively, or non-invasively. In some cases, an epidermal sample may be obtained at least two of invasively, semi-invasively, or non-invasively. In some cases, an epidermal sample may be obtained invasively. In some cases, an epidermal sample may be obtained semi-invasively. In some cases, an epidermal sample may be obtained non-invasively. In some cases, an epidermal sample may be obtained invasively, semi-invasively, and non-invasively.

In some instances, non-invasive epidermal sampling may facilitate sampling in non-clinical settings (e.g., such as a home, a store, or an office). In other cases, because non-invasive epidermal sampling does not cause an injury to a subject being sampled, non-invasive epidermal sampling may facilitate multiple samplings. In some cases, non-invasive epidermal sampling may reduce or minimize adverse effects in the subject being sampled. Such an adverse effect may comprise infection or injury. In some cases, non-invasive epidermal sampling extract cell types from a skin of a subject different from the cell types extracted by invasive methods. In some cases, non-invasive sampling may not extract Langerhans cell, Merkel cell, or a combination thereof from a skin of a subject. In some cases, non-invasive sampling may not extract Langerhans cell from a skin of a subject. In some cases, non-invasive sampling may not extract Merkel cell from a skin of a subject,

In some cases, non-invasive epidermal sampling may comprise using an adhesive article to remove an epidermal sample. In some cases, non-invasive epidermal sampling may comprise applying an adhesive article to a skin area of the subject. In some cases, non-invasive epidermal sampling may comprise a portion of skin tissues or cells from the skin area of the subject being applied with the adhesive article. In some cases, the adhesive article may comprise a skin patch. In some cases, the skin patch may be D-Squame™ skin patch.

In some instances, the skin patch may comprise various material. In some cases, the skin patch may comprise an acrylic-based adhesive. In some cases, a skin patch may have a peak force of about 0.01 Newtons, about 0.05 Newtons, about 0.1 Newtons, about 0.2 Newtons, about 0.3 Newtons, about 0.4 Newtons, about 0.5 Newtons, about 0.6 Newtons, about 0.7 Newtons, about 0.8 Newtons, about 0.9 Newtons, about 1 Newtons, about 1.1 Newtons, about 1.2 Newtons, about 1.3 Newtons, about 1.4 Newtons, about 1.5 Newtons, about 1.6 Newtons, about 1.7 Newtons, about 1.8 Newtons, about 1.9 Newtons, about 2 Newtons, about 2.1 Newtons, about 2.2 Newtons, about 2.3 Newtons, about 2.4 Newtons, about 2.5 Newtons, about 2.6 Newtons, about 2.7 Newtons, about 2.8 Newtons, about 2.9 Newtons, about 3 Newtons, about 3.5 Newtons, about 4 Newtons, about 4.5 Newtons, about 5 Newtons, about 5.5 Newtons, about 6 Newtons, about 6.5 Newtons, about 7 Newtons, about 7.5 Newtons, about 8 Newtons, about 8.5 Newtons, about 9 Newtons, about 9.5 Newtons, about 10 Newtons, or more. In some cases, a skin patch may have an adhesion, when measured by a texture analyzer, of about 1×10\hat{ }-7 Newton meters, about 5×10\hat{ }-7 Newton meters, about 1×10\hat{ }-6 Newton meters, about 5×10\hat{ }-6 Newton meters, about 1×10\hat{ }-5 Newton meters, about 5×10\hat{ }-5 Newton meters, about 1×10\hat{ }-4 Newton meters, about 5×10\hat{ }-4 Newton meters, about 1×10\hat{ }-3 Newton meters, about 5×10\hat{ }-3 Newton meters, about 1×10\hat{ }-2 Newton meters, about 5×10\hat{ }-2 Newton meters, about 1×10\hat{ }-1 Newton meters, about 5×10\hat{ }-1 Newton meters, or more.

In some case, the adhesive article may also comprise a rubber-based adhesive. In some case, the adhesive article may also comprise a rubber compound. In some instances, the adhesive article may comprise adhesive matrix material. In some cases, the adhesive article may comprise a latex material, a silicone material, or a combination thereof. In other cases, the adhesive article may comprise a matrix. In some cases, the adhesive article may not comprise a latex material, a silicone material, or a combination thereof. In some cases, the adhesive article comprises a polyurethane film.

In some instances, the adhesive article may comprise a flexible material. In some cases, the adhesive article may adapt the shape of a skin surface upon application of the skin surface. In some cases, at least the first collection area is flexible.

In some cases, the matrix material may be able to adhere to a skin surface. In other cases, the matrix material may not cause a break to the skin surface. In some cases, such a break may cause injury to the skin. In some instances, the adhesive matrix material may not produce any residues on the skin surface after the application of the adhesive matrix. In some instances, the adhesive matrix material may not produce any residues on the skin surface after the removal of the adhesive matrix. In some cases, the adhesive matrix material may not cause irritation to the skin surface that application of the adhesive matrix. In some cases, the adhesive matrix material may comprise a transparent material. In some cases, the skin area with an adhesive article applied may be visible.

In some cases, non-invasive epidermal sampling may comprise applying an adhesive article to a skin area of the subject. In some cases, non-invasive epidermal sampling may comprise applying at least one adhesive article to a skin area of the subject. In some cases, non-invasive epidermal sampling may comprise applying more than one adhesive article to a skin area of the subject. In some cases, non-invasive epidermal sampling may comprise applying 2 adhesive articles to a skin area of the subject. In some cases, non-invasive epidermal sampling may comprise applying 3 adhesive articles to a skin area of the subject. In some cases, non-invasive epidermal sampling may comprise applying 4 adhesive articles to a skin area of the subject. In some cases, non-invasive epidermal sampling may comprise applying 5 adhesive articles to a skin area of the subject. In some cases, non-invasive epidermal sampling may comprise applying 6 adhesive articles to a skin area of the subject. In some cases, non-invasive epidermal sampling may comprise applying 7 adhesive articles to a skin area of the subject. In some cases, non-invasive epidermal sampling may comprise applying 8 adhesive articles to a skin area of the subject. In some cases, non-invasive epidermal sampling may comprise applying 9 adhesive articles to a skin area of the subject. In some cases, non-invasive epidermal sampling may comprise applying 10 adhesive articles to a skin area of the subject. In some cases, non-invasive epidermal sampling may comprise applying 11 adhesive articles to a skin area of the subject. In some cases, non-invasive epidermal sampling may comprise applying 12 adhesive articles to a skin area of the subject. In some cases, non-invasive epidermal sampling may comprise applying 13 adhesive articles to a skin area of the subject. In some cases, non-invasive epidermal sampling may comprise applying 14 adhesive articles to a skin area of the subject. In some cases, non-invasive epidermal sampling may comprise applying 15 adhesive articles to a skin area of the subject. In some cases, non-invasive epidermal sampling may comprise applying 16 adhesive articles to a skin area of the subject. In some cases, non-invasive epidermal sampling may comprise applying 17 adhesive articles to a skin area of the subject. In some cases, non-invasive epidermal sampling may comprise applying 18 adhesive articles to a skin area of the subject. In some cases, non-invasive epidermal sampling may comprise applying 19 adhesive articles to a skin area of the subject. In some cases, non-invasive epidermal sampling may comprise applying 20 adhesive articles to a skin area of the subject. In some cases, non-invasive epidermal sampling may comprise applying at least, or more than 20 adhesive articles to a skin area of the subject. In some cases, non-invasive epidermal sampling may comprise applying from 1 to 20 adhesive articles to a skin area of the subject. In some cases, non-invasive epidermal sampling may comprise applying from 1 to 15 adhesive articles to a skin area of the subject. In some cases, non-invasive epidermal sampling may comprise applying from 1 to 10 adhesive articles to a skin area of the subject. In some cases, non-invasive epidermal sampling may comprise applying from 1 to 9 adhesive articles to a skin area of the subject. In some cases, non-invasive epidermal sampling may comprise applying from 1 to 8 adhesive articles to a skin area of the subject. In some cases, non-invasive epidermal sampling may comprise applying from 2 to 8 adhesive articles to a skin area of the subject. In some cases, non-invasive epidermal sampling may comprise applying from 3 to 8 adhesive articles to a skin area of the subject. In some cases, non-invasive epidermal sampling may comprise applying from 4 to 8 adhesive articles to a skin area of the subject. In some cases, non-invasive epidermal sampling may comprise applying from 5 to 8 adhesive articles to a skin area of the subject. In some cases, non-invasive epidermal sampling may comprise applying from 6 to 8 adhesive articles to a skin area of the subject. In some cases, non-invasive epidermal sampling may comprise applying from 7 to 8 adhesive articles to a skin area of the subject.

In some instances, an adhesive article is applied on a skin surface. In some cases, an adhesive article is applied to a skin surface located on the area comprising the abdomen, arm, back, chest, elbow, face, finger, foot, hand, hip, leg, neck, palm, shoulder, thigh, toe, or a combination thereof. In some cases, an adhesive article is applied to a skin surface located on the area comprising at least two of the abdomen, arm, back, chest, elbow, face, finger, foot, hand, hip, leg, neck, palm, shoulder, thigh, or toe. In some embodiments, the skin surface is not ulcerated, bleeding, or injured. In some cases, an adhesive article is applied to a skin surface located on the area comprising abdomen. In some cases, an adhesive article is applied to a skin surface located on the area comprising arm. In some cases, an adhesive article is applied to a skin surface located on the area comprising back. In some cases, an adhesive article is applied to a skin surface located on the area comprising chest. In some cases, an adhesive article is applied to a skin surface located on the area comprising elbow. In some cases, an adhesive article is applied to a skin surface located on the area comprising face. In some cases, an adhesive article is applied to a skin surface located on the area comprising finger. In some cases, an adhesive article is applied to a skin surface located on the area comprising foot. In some cases, an adhesive article is applied to a skin surface located on the area comprising hand. In some cases, an adhesive article is applied to a skin surface located on the area comprising hip. In some cases, an adhesive article is applied to a skin surface located on the area comprising leg. In some cases, an adhesive article is applied to a skin surface located on the area comprising neck. In some cases, an adhesive article is applied to a skin surface located on the area comprising palm. In some cases, an adhesive article is applied to a skin surface located on the area comprising shoulder. In some cases, an adhesive article is applied to a skin surface located on the area comprising thigh. In some cases, an adhesive article is applied to a skin surface located on the area comprising toe. In some embodiments, the skin surface has not been previously biopsied.

In some cases, the adhesion article is applied to a face skin surface area In some cases, a face skin surface area may comprise forehead, nose, eyebrow area, cheek, hairline, ear, jawline, chin, or a combination thereof. In some cases, a face skin surface area may comprise at least two of forehead, nose, eyebrow area, cheek, hairline, ear, jawline, or chin. In some cases, a face skin surface area may comprise cheek. In some cases, a face skin surface area may comprise chin. In some cases, a face skin surface area may comprise ear. In some cases, a face skin surface area may comprise eyebrow area. In some cases, a face skin surface area may comprise forehead. In some cases, a face skin surface area may comprise hairline. In some cases, a face skin surface area may comprise jawline. In some cases, a face skin surface area may comprise nose.

In some instances, an adhesive article may have a circular shape. In some cases, an adhesive article may have an oval shape. In some cases, an adhesive article may have a polygon shape. The polygon shape of an adhesive article may be a triangle, rectangle, square, pentagon, hexagon, heptagon, octagon, nonagon, or decagon. In some cases, the polygon shape may comprise 3, 4, 5, 6, 7, 8, 9, 10, 11, 12, 13, 14, 15, 16, 17, 18, 19, 20, or more sides or bounding outer line segments. In some cases, the length of the sides or bounding outer line segments of an adhesive article be the same or different.

In some instance, a circular shaped adhesive article may have a diameter of about 5 mm (millimeter), about 10 mm, about 15 mm, about 25 mm, about 30 mm, about 35 mm, about 40 mm, about 55 mm, about 60 mm, about 65 mm, about 70 mm, about 75 mm, about 80 mm, about 85 mm, about 90 mm, about 95 mm, about 100 mm, or greater than 100 mm. In some instance, a circular shaped adhesive article may have a diameter from about 5 mm to 50 mm, from about 10 mm to 45 mm, from about 15 mm to 40 mm, from about 25 mm to 35 mm, from about 5 mm to 50 mm, from about 10 mm to 50 mm, from about 15 mm to 50 mm, from about 20 mm to 50 mm, or from about 25 mm to 50 mm.

In some instances, the length of the sides or bound outer line segments of a polygon shaped adhesive article may be about 5 mm (millimeter), about 10 mm, about 15 mm, about 25 mm, about 30 mm, about 35 mm, about 40 mm, about 55 mm, about 60 mm, about 65 mm, about 70 mm, about 75 mm, about 80 mm, about 85 mm, about 90 mm, about 95 mm, about 100 mm, or greater than 100 mm. In some instance, a circular shaped adhesive article may have a diameter from about 5 mm to 50 mm, from about 10 mm to 45 mm, from about 15 mm to 40 mm, from about 25 mm to 35 mm, from about 5 mm to 50 mm, from about 10 mm to 50 mm, from about 15 mm to 50 mm, from about 20 mm to 50 mm, or from about 25 mm to 50 mm.

In some instances, the thickness of an adhesive article may be about 0.05 mil, about 0.1 mil, about 0.2 mil, about 0.3 mil, about 0.4 mil, about 0.5 mil, about 0.6 mil, about 0.7 mil, about 0.8 mil, about 0.9 mil, about 1 mil, about 1.5 mil, about 2 mil, about 2.5 mil, about 3 mil, about 3.5 mil, about 4 mil, about 4.5 mil, about 5 mil, about 5.5 mil, about 6 mil, about 6.5 mil, about 7 mil, about 7.5 mil, about 8 mil, about 8.5 mil, about 9 mil, about 9.5 mil, about 10 mil, or more than 10 mil. In some cases, the thickness of an adhesive article may be from about 0.05 mil to about 10 mil, from about 0.1 mil to about 9.5 mil, from about 0.2 mil to about 9 mil, from about 0.3 mil to about 8.5 mil, from about 0.4 mil to about 8 mil, from about 0.5 mil to about 7.5 mil, from about 0.6 mil to about 7 mil, from about 0.7 mil to about 6.5 mil, from about 0.8 mil to about 6 mil, from about 0.9 mil to about 5.5 mil, or from about 1 mil to about 5 mil.

In some cases, the area of the adhesive article may be from about 250 mm² to about 2000 mm², from about 250 mm² to about 1750 mm², from about 250 mm² to about 1500 mm², from about 250 mm² to about 1250 mm², from about 250 mm² to about 1000 mm², from about 500 mm² to about 1000 mm², from about 500 mm² to about 1250 mm², from about 500 mm² to about 1500 mm², from about 500 mm² to about 1750 mm², or from about 500 mm² to about 2000 mm². In some instances, the area of the adhesive article may be about 250 mm², about 500 mm², about 750 mm², about 1000 mm², about 1250 mm², about 1500 mm², about 1750 mm², about 2000 mm², or more than about 2000 mm², about 2250 mm², about 2500 mm², about 2750 mm², about 3000 mm².

In some instances, the length of the sides or bounding outer line segments of a carrier sheet may be about 50 mm, about 55 mm, about 60 mm, about 65 mm, about 70 mm, about 75 mm, about 80 mm, about 85 mm, about 90 mm, about 95 mm, about 100 mm, about 105 mm, about 110 mm, about 115 mm, about 120 mm, about 125 mm, about 130 mm, about 135 mm, about 140 mm, about 145 mm, about 150 mm, about 155 mm, about 160 mm, about 165 mm, about 170 mm, about 175 mm, about 180 mm, about 185 mm, about 190 mm, about 195 mm, about 200 mm, or more than 200 mm.

In some instances, an adhesive article may be deposited on top of a carrier sheet. In some instances, the outer boundary of the adhesive article may be surrounded or bound by a carrier sheet.

In some cases, a carrier sheet may be composed of a polymer. In some cases, a carrier sheet may have a polygon shape. The polygon shape of a carrier sheet may be a triangle, rectangle, square, pentagon, hexagon, heptagon, octagon, nonagon, or decagon. In some cases, the polygon shape of a carrier sheet may comprise 3, 4, 5, 6, 7, 8, 9, 10, 11, 12, 13, 14, 15, 16, 17, 18, 19, 20, or more sides or bounding outer line segments. In some cases, the length of the sides or bounding outer line segments of a carrier sheet be the same or different. In some cases, the carrier sheet may be circular shaped or oval shaped.

In some instances, the carrier sheet may function as a tab. In some cases, the tab may be used for applying and removing the adhesive article attached to the carrier sheet. In some cases, the tab may be sufficient in size so that while applying the adhesive patch to a skin surface, the applicant does not come in contact with the adhesive tape. In some cases, the adhesive may not contain a carrier sheet.

In some cases, the area of a carrier sheet may be about 4000 mm², about 5000 mm², about 6000 mm², about 7000 mm², about 8000 mm², about 9000 mm², about 10000 mm², about 11000 mm², about 12000 mm², about 13000 mm², about 14000 mm², about 15000 mm², about 16000 mm², about 17000 mm², about 18000 mm², about 19000 mm², about 20000 mm², or more than 20000 mm². In some cases, the area of a carrier sheet may be from about 7000 mm² to about 15000 mm², from about 8000 mm² to about 15000 mm², from about 9000 mm² to about 15000 mm², from about 10000 mm² to about 15000 mm², from about 11000 mm² to about 15000 mm², from about 12000 mm² to about 15000 mm², from about 12000 mm² to about 16000 mm², from about 12000 mm² to about 17000 mm², from about 12000 mm² to about 18000 mm², from about 12000 mm² to about 19000 mm², or from about 12000 mm² to about 20000 mm².

In some instances, non-invasive epidermal sampling may yield about 0.1 nanograms (ng), about 0.2 ng, about 0.3 ng, about 0.4 ng, about 0.5 ng, about 0.6 ng, about 0.7 ng, about 0.8 ng, about 0.9 ng, about 1 ng, about 2 ng, about 3 ng, about 4 ng, about 5 ng, about 6 ng, about 7 ng, about 8 ng, about 9 ng, about 10 ng, about 15 ng, about 20 ng, about 25 ng, about 30 ng, about 35 ng, about 40 ng, about 45 ng, about 50 ng, about 55 ng, about 60 ng, about 65 ng, about 70 ng, about 75 ng, about 80 ng, about 85 ng, about 90 ng, about 95 ng, about 100 ng, about 500 ng, about 1000 ng, or more than 1000 ng nucleic acids. In some cases, non-invasive epidermal sampling may yield from about 0.1 ng to about 1000 ng, from about 0.5 ng to about 500 ng, from about 1 ng to about 100 ng, from about 5 ng to about 90 ng, from about 10 ng to about 80 ng, from about 15 ng to about 70 ng, from about 20 ng to about 60 ng, from about 20 ng to about 55 ng, from about 21 ng to about 54 ng, from about 22 ng to about 53 ng, from about 23 ng to about 52 ng, from about 24 ng to about 51 ng, or from about 25 ng to about 50 ng nucleic acids.

In some instances, non-invasive epidermal sampling may yield about 0.1 nanograms (ng), about 0.2 ng, about 0.3 ng, about 0.4 ng, about 0.5 ng, about 0.6 ng, about 0.7 ng, about 0.8 ng, about 0.9 ng, about 1 ng, about 2 ng, about 3 ng, about 4 ng, about 5 ng, about 6 ng, about 7 ng, about 8 ng, about 9 ng, about 10 ng, about 15 ng, about 20 ng, about 25 ng, about 30 ng, about 35 ng, about 40 ng, about 45 ng, about 50 ng, about 55 ng, about 60 ng, about 65 ng, about 70 ng, about 75 ng, about 80 ng, about 85 ng, about 90 ng, about 95 ng, about 100 ng, about 500 ng, about 1000 ng, or more than 1000 ng DNA or RNA. In some cases, non-invasive epidermal sampling may yield from about 0.1 ng to about 1000 ng, from about 0.5 ng to about 500 ng, from about 1 ng to about 100 ng, from about 5 ng to about 90 ng, from about 10 ng to about 80 ng, from about 15 ng to about 70 ng, from about 20 ng to about 60 ng, from about 20 ng to about 55 ng, from about 21 ng to about 54 ng, from about 22 ng to about 53 ng, from about 23 ng to about 52 ng, from about 24 ng to about 51 ng, or from about 25 ng to about 50 ng DNA or RNA.

In some instances, non-invasive epidermal sampling may yield about 0.1 nanograms (ng), about 0.2 ng, about 0.3 ng, about 0.4 ng, about 0.5 ng, about 0.6 ng, about 0.7 ng, about 0.8 ng, about 0.9 ng, about 1 ng, about 2 ng, about 3 ng, about 4 ng, about 5 ng, about 6 ng, about 7 ng, about 8 ng, about 9 ng, about 10 ng, about 15 ng, about 20 ng, about 25 ng, about 30 ng, about 35 ng, about 40 ng, about 45 ng, about 50 ng, about 55 ng, about 60 ng, about 65 ng, about 70 ng, about 75 ng, about 80 ng, about 85 ng, about 90 ng, about 95 ng, about 100 ng, about 500 ng, about 1000 ng, or more than 1000 ng DNA. In some cases, non-invasive epidermal sampling may yield from about 0.1 ng to about 1000 ng, from about 0.5 ng to about 500 ng, from about 1 ng to about 100 ng, from about 5 ng to about 90 ng, from about 10 ng to about 80 ng, from about 15 ng to about 70 ng, from about 20 ng to about 60 ng, from about 20 ng to about 55 ng, from about 21 ng to about 54 ng, from about 22 ng to about 53 ng, from about 23 ng to about 52 ng, from about 24 ng to about 51 ng, or from about 25 ng to about 50 ng DNA.

In some instances, non-invasive epidermal sampling may yield about 0.1 nanograms (ng), about 0.2 ng, about 0.3 ng, about 0.4 ng, about 0.5 ng, about 0.6 ng, about 0.7 ng, about 0.8 ng, about 0.9 ng, about 1 ng, about 2 ng, about 3 ng, about 4 ng, about 5 ng, about 6 ng, about 7 ng, about 8 ng, about 9 ng, about 10 ng, about 15 ng, about 20 ng, about 25 ng, about 30 ng, about 35 ng, about 40 ng, about 45 ng, about 50 ng, about 55 ng, about 60 ng, about 65 ng, about 70 ng, about 75 ng, about 80 ng, about 85 ng, about 90 ng, about 95 ng, about 100 ng, about 500 ng, about 1000 ng, or more than 1000 ng RNA. In some cases, non-invasive epidermal sampling may yield from about 0.1 ng to about 1000 ng, from about 0.5 ng to about 500 ng, from about 1 ng to about 100 ng, from about 5 ng to about 90 ng, from about 10 ng to about 80 ng, from about 15 ng to about 70 ng, from about 20 ng to about 60 ng, from about 20 ng to about 55 ng, from about 21 ng to about 54 ng, from about 22 ng to about 53 ng, from about 23 ng to about 52 ng, from about 24 ng to about 51 ng, or from about 25 ng to about 50 ng RNA.

In some instances, non-invasive epidermal sampling may yield about 0.1 nanograms (ng), about 0.2 ng, about 0.3 ng, about 0.4 ng, about 0.5 ng, about 0.6 ng, about 0.7 ng, about 0.8 ng, about 0.9 ng, about 1 ng, about 2 ng, about 3 ng, about 4 ng, about 5 ng, about 6 ng, about 7 ng, about 8 ng, about 9 ng, about 10 ng, about 15 ng, about 20 ng, about 25 ng, about 30 ng, about 35 ng, about 40 ng, about 45 ng, about 50 ng, about 55 ng, about 60 ng, about 65 ng, about 70 ng, about 75 ng, about 80 ng, about 85 ng, about 90 ng, about 95 ng, about 100 ng, about 500 ng, about 1000 ng, or more than 1000 ng DNA and RNA. In some cases, non-invasive epidermal sampling may yield from about 0.1 ng to about 1000 ng, from about 0.5 ng to about 500 ng, from about 1 ng to about 100 ng, from about 5 ng to about 90 ng, from about 10 ng to about 80 ng, from about 15 ng to about 70 ng, from about 20 ng to about 60 ng, from about 20 ng to about 55 ng, from about 21 ng to about 54 ng, from about 22 ng to about 53 ng, from about 23 ng to about 52 ng, from about 24 ng to about 51 ng, or from about 25 ng to about 50 DNA and RNA.

In some instances, invasive epidermal sampling may comprise a biopsy. In some cases, invasive epidermal sampling may comprise a punch biopsy. In some instances, invasive epidermal sampling may yield about 0.1 ng, 0.5 ng, 1 ng, 5 ng, 10 ng, 50 ng, 100 ng, 500 ng, 1000 ng, 5000 ng, 10000 ng, 50000 ng, 100000 ng, 500000 ng, 1000000 ng, or 1000000 ng nucleic acids.

### Epidermal Sampling Kit

In some instances, the adhesive article is provided on a detachable release sheet in an epidermal sampling kit. In some cases, the adhesive article is provided on a removable release sheet in an epidermal sampling kit. In some cases, the adhesive article is provided on a strippable release sheet in an epidermal sampling kit.

In some instances, the adhesive article in an epidermal sampling kit may be stable at a temperature of about −80° C., about −70° C., about −60° C., about −50° C., about −40° C., about-30° C., about −20° C., about −10° C., about 0° C., about 10° C., about 20° C., or about 30° C. In some cases, the adhesive article in an epidermal sampling kit may be stable at a temperature of about −80° C. In some cases, the adhesive article in an epidermal sampling kit may be stable at a temperature of about −70° C. In some cases, the adhesive article in an epidermal sampling kit may be stable at a temperature of about −60° C. In some cases, the adhesive article in an epidermal sampling kit may be stable at a temperature of about −50° C. In some cases, the adhesive article in an epidermal sampling kit may be stable at a temperature of about −40° C. In some cases, the adhesive article in an epidermal sampling kit may be stable at a temperature of about −30° C. In some cases, the adhesive article in an epidermal sampling kit may be stable at a temperature of about −20° C. In some cases, the adhesive article in an epidermal sampling kit may be stable at a temperature of about −10° C. In some cases, the adhesive article in an epidermal sampling kit may be stable at a temperature of about 0° C. In some cases, the adhesive article in an epidermal sampling kit may be stable at a temperature of about 10° C. In some cases, the adhesive article in an epidermal sampling kit may be stable at a temperature of about 20° C. In some cases, the adhesive article in an epidermal sampling kit may be stable at a temperature of about 30° C.

In some cases, the adhesive article in an epidermal sampling kit may be stable at a temperature from about −80° C. to about 30° C. In some cases, the adhesive article in an epidermal sampling kit may be stable at a temperature of about −70° C. to about 30° C. In some cases, the adhesive article in an epidermal sampling kit may be stable at a temperature of about −60° C. to about 30° C. In some cases, the adhesive article in an epidermal sampling kit may be stable at a temperature of about −50° C. to about 30° C. In some cases, the adhesive article in an epidermal sampling kit may be stable at a temperature of about −40° C. to about 30° C. In some cases, the adhesive article in an epidermal sampling kit may be stable at a temperature of about −30° C. to about 30° C. In some cases, the adhesive article in an epidermal sampling kit may be stable at a temperature of about −20° C. to about 30° C. In some cases, the adhesive article in an epidermal sampling kit may be stable at a temperature of about −10° C. to about 30° C. In some cases, the adhesive article in an epidermal sampling kit may be stable at a temperature of about 0° C. to about 30° C. In some cases, the adhesive article in an epidermal sampling kit may be stable at a temperature of about 4° C. to about 30° C. In some cases, the adhesive article in an epidermal sampling kit may be stable at a temperature of about 10° C. to about 30° C.

In some instances, the adhesive article in an epidermal sampling kit may be stable for at least about 0.5 years, about 1 years, about 2 years, about 3 years, about 4 years, about 5 years, about 6 years, about 7 years, about 8 years, about 9 years, about 10 years, about 11 years, about 12 years, about 13 years, about 14 years, or about 15 years.

In some instances, an epidermal sampling kit may comprise at least about 1 adhesive article, about 2 adhesive articles, about 3 adhesive articles, about 4 adhesive articles, about 5 adhesive articles, about 6 adhesive articles, about 7 adhesive articles, about 8 adhesive articles, about 9 adhesive articles, about 10 adhesive articles, about 11 adhesive articles, about 12 adhesive articles, about 13 adhesive articles, about 14 adhesive articles, about 15 adhesive articles, about 16 adhesive articles, about 17 adhesive articles, about 18 adhesive articles, about 19 adhesive articles, about 20 adhesive articles, about 21 adhesive articles, about 22 adhesive articles, about 23 adhesive articles, about 24 adhesive articles, about 25 adhesive articles, about 26 adhesive articles, about 27 adhesive articles, about 28 adhesive articles, about 29 adhesive articles, about 30 adhesive articles, about 31 adhesive articles, about 32 adhesive articles, about 33 adhesive articles, about 34 adhesive articles, about 35 adhesive articles, about 36 adhesive articles, about 37 adhesive articles, about 38 adhesive articles, about 39 adhesive articles, about 40 adhesive articles, about 41 adhesive articles, about 42 adhesive articles, about 43 adhesive articles, about 44 adhesive articles, about 45 adhesive articles, about 46 adhesive articles, about 47 adhesive articles, about 48 adhesive articles, about 49 adhesive articles, about 50 adhesive articles, about 60 adhesive articles, about 70 adhesive articles, about 80 adhesive articles, about 90 adhesive articles, about 100 adhesive articles, or more than 100 adhesive articles.

In some cases, an epidermal sampling kit may comprise at least about 1 adhesive articles. In some cases, an epidermal sampling kit may comprise at least about 2 adhesive articles. In some cases, an epidermal sampling kit may comprise at least about 3 adhesive articles. In some cases, an epidermal sampling kit may comprise at least about 4 adhesive articles. In some cases, an epidermal sampling kit may comprise at least about 5 adhesive articles. In some cases, an epidermal sampling kit may comprise at least about 6 adhesive articles. In some cases, an epidermal sampling kit may comprise at least about 7 adhesive articles. In some cases, an epidermal sampling kit may comprise at least about 8 adhesive articles. In some cases, an epidermal sampling kit may comprise at least about 9 adhesive articles. In some cases, an epidermal sampling kit may comprise at least about 10 adhesive articles. In some cases, an epidermal sampling kit may comprise at least about 11 adhesive articles. In some cases, an epidermal sampling kit may comprise at least about 12 adhesive articles. In some cases, an epidermal sampling kit may comprise at least about 13 adhesive articles. In some cases, an epidermal sampling kit may comprise at least about 14 adhesive articles. In some cases, an epidermal sampling kit may comprise at least about 15 adhesive articles. In some cases, an epidermal sampling kit may comprise at least about 16 adhesive articles. In some cases, an epidermal sampling kit may comprise at least about 17 adhesive articles. In some cases, an epidermal sampling kit may comprise at least about 18 adhesive articles. In some cases, an epidermal sampling kit may comprise at least about 19 adhesive articles. In some cases, an epidermal sampling kit may comprise at least about 20 adhesive articles. In some cases, an epidermal sampling kit may comprise at least about 21 adhesive articles. In some cases, an epidermal sampling kit may comprise at least about 22 adhesive articles. In some cases, an epidermal sampling kit may comprise at least about 23 adhesive articles. In some cases, an epidermal sampling kit may comprise at least about 24 adhesive articles. In some cases, an epidermal sampling kit may comprise at least about 25 adhesive articles. In some cases, an epidermal sampling kit may comprise at least about 26 adhesive articles. In some cases, an epidermal sampling kit may comprise at least about 27 adhesive articles. In some cases, an epidermal sampling kit may comprise at least about 28 adhesive articles. In some cases, an epidermal sampling kit may comprise at least about 29 adhesive articles. In some cases, an epidermal sampling kit may comprise at least about 30 adhesive articles. In some cases, an epidermal sampling kit may comprise at least about 31 adhesive articles. In some cases, an epidermal sampling kit may comprise at least about 32 adhesive articles. In some cases, an epidermal sampling kit may comprise at least about 33 adhesive articles. In some cases, an epidermal sampling kit may comprise at least about 34 adhesive articles. In some cases, an epidermal sampling kit may comprise at least about 35 adhesive articles. In some cases, an epidermal sampling kit may comprise at least about 36 adhesive articles. In some cases, an epidermal sampling kit may comprise at least about 37 adhesive articles. In some cases, an epidermal sampling kit may comprise at least about 38 adhesive articles. In some cases, an epidermal sampling kit may comprise at least about 39 adhesive articles. In some cases, an epidermal sampling kit may comprise at least about 40 adhesive articles. In some cases, an epidermal sampling kit may comprise at least about 41 adhesive articles. In some cases, an epidermal sampling kit may comprise at least about 42 adhesive articles. In some cases, an epidermal sampling kit may comprise at least about 43 adhesive articles. In some cases, an epidermal sampling kit may comprise at least about 44 adhesive articles. In some cases, an epidermal sampling kit may comprise at least about 45 adhesive articles. In some cases, an epidermal sampling kit may comprise at least about 46 adhesive articles. In some cases, an epidermal sampling kit may comprise at least about 47 adhesive articles. In some cases, an epidermal sampling kit may comprise at least about 48 adhesive articles. In some cases, an epidermal sampling kit may comprise at least about 49 adhesive articles. In some cases, an epidermal sampling kit may comprise at least about 50 adhesive articles. In some cases, an epidermal sampling kit may comprise at least about 60 adhesive articles. In some cases, an epidermal sampling kit may comprise at least about 70 adhesive articles. In some cases, an epidermal sampling kit may comprise at least about 80 adhesive articles. In some cases, an epidermal sampling kit may comprise at least about 90 adhesive articles. In some cases, an epidermal sampling kit may comprise at least about 100 adhesive articles.

In some cases, an epidermal sampling kit may comprise at most about 1 adhesive articles. In some cases, an epidermal sampling kit may comprise at most about 2 adhesive articles. In some cases, an epidermal sampling kit may comprise at most about 3 adhesive articles. In some cases, an epidermal sampling kit may comprise at most about 4 adhesive articles. In some cases, an epidermal sampling kit may comprise at most about 5 adhesive articles. In some cases, an epidermal sampling kit may comprise at most about 6 adhesive articles. In some cases, an epidermal sampling kit may comprise at most about 7 adhesive articles. In some cases, an epidermal sampling kit may comprise at most about 8 adhesive articles. In some cases, an epidermal sampling kit may comprise at most about 9 adhesive articles. In some cases, an epidermal sampling kit may comprise at most about 10 adhesive articles. In some cases, an epidermal sampling kit may comprise at most about 11 adhesive articles. In some cases, an epidermal sampling kit may comprise at most about 12 adhesive articles. In some cases, an epidermal sampling kit may comprise at most about 13 adhesive articles. In some cases, an epidermal sampling kit may comprise at most about 14 adhesive articles. In some cases, an epidermal sampling kit may comprise at most about 15 adhesive articles. In some cases, an epidermal sampling kit may comprise at most about 16 adhesive articles. In some cases, an epidermal sampling kit may comprise at most about 17 adhesive articles. In some cases, an epidermal sampling kit may comprise at most about 18 adhesive articles. In some cases, an epidermal sampling kit may comprise at most about 19 adhesive articles. In some cases, an epidermal sampling kit may comprise at most about 20 adhesive articles. In some cases, an epidermal sampling kit may comprise at most about 21 adhesive articles. In some cases, an epidermal sampling kit may comprise at most about 22 adhesive articles. In some cases, an epidermal sampling kit may comprise at most about 23 adhesive articles. In some cases, an epidermal sampling kit may comprise at most about 24 adhesive articles. In some cases, an epidermal sampling kit may comprise at most about 25 adhesive articles. In some cases, an epidermal sampling kit may comprise at most about 26 adhesive articles. In some cases, an epidermal sampling kit may comprise at most about 27 adhesive articles. In some cases, an epidermal sampling kit may comprise at most about 28 adhesive articles. In some cases, an epidermal sampling kit may comprise at most about 29 adhesive articles. In some cases, an epidermal sampling kit may comprise at most about 30 adhesive articles. In some cases, an epidermal sampling kit may comprise at most about 31 adhesive articles. In some cases, an epidermal sampling kit may comprise at most about 32 adhesive articles. In some cases, an epidermal sampling kit may comprise at most about 33 adhesive articles. In some cases, an epidermal sampling kit may comprise at most about 34 adhesive articles. In some cases, an epidermal sampling kit may comprise at most about 35 adhesive articles. In some cases, an epidermal sampling kit may comprise at most about 36 adhesive articles. In some cases, an epidermal sampling kit may comprise at most about 37 adhesive articles. In some cases, an epidermal sampling kit may comprise at most about 38 adhesive articles. In some cases, an epidermal sampling kit may comprise at most about 39 adhesive articles. In some cases, an epidermal sampling kit may comprise at most about 40 adhesive articles. In some cases, an epidermal sampling kit may comprise at most about 41 adhesive articles. In some cases, an epidermal sampling kit may comprise at most about 42 adhesive articles. In some cases, an epidermal sampling kit may comprise at most about 43 adhesive articles. In some cases, an epidermal sampling kit may comprise at most about 44 adhesive articles. In some cases, an epidermal sampling kit may comprise at most about 45 adhesive articles. In some cases, an epidermal sampling kit may comprise at most about 46 adhesive articles. In some cases, an epidermal sampling kit may comprise at most about 47 adhesive articles. In some cases, an epidermal sampling kit may comprise at most about 48 adhesive articles. In some cases, an epidermal sampling kit may comprise at most about 49 adhesive articles. In some cases, an epidermal sampling kit may comprise at most about 50 adhesive articles. In some cases, an epidermal sampling kit may comprise at most about 60 adhesive articles. In some cases, an epidermal sampling kit may comprise at most about 70 adhesive articles. In some cases, an epidermal sampling kit may comprise at most about 80 adhesive articles. In some cases, an epidermal sampling kit may comprise at most about 90 adhesive articles. In some cases, an epidermal sampling kit may comprise at most about 100 adhesive articles.

In some cases, an epidermal sampling kit may comprise from about 1 to about 16 adhesive articles, from about 2 to about 15 adhesive articles, from about 3 to about 14 adhesive articles, from about 4 to about 13 adhesive articles, from about 5 to about 12 adhesive articles, from about 6 to about 11 adhesive articles, from about 7 to about 10 adhesive articles, or from about 8 to about 9 adhesive articles. In some cases, an epidermal sampling kit may comprise from about 1 to about 16 adhesive articles. In some cases, an epidermal sampling kit may comprise from about 2 to about 15 adhesive articles. In some cases, an epidermal sampling kit may comprise from about 3 to about 14 adhesive articles. In some cases, an epidermal sampling kit may comprise from about 4 to about 13 adhesive articles. In some cases, an epidermal sampling kit may comprise from about 5 to about 12 adhesive articles. In some cases, an epidermal sampling kit may comprise from about 6 to about 11 adhesive articles. In some cases, an epidermal sampling kit may comprise from about 7 to about 10 adhesive articles. In some cases, an epidermal sampling kit may comprise from about 8 to about 9 adhesive articles.

In some instances, an adhesive article may have an epidermal sampling area. In some cases, an epidermal sampling area may be the area of the adhesive article that can contact a skin. In some cases, an epidermal sampling area may be the area of the adhesive article that contacts a skin. In some cases, an epidermal sampling area may be the area of the adhesive article that can collect an epidermal sample from a skin. In some cases, an epidermal sampling area may be the area of the adhesive article that collects an epidermal sample from a skin. In some cases, an epidermal sampling area may be the area of the adhesive article that can extract an epidermal sample from a skin. In some cases, an epidermal sampling area may be the area of the adhesive article that extracts an epidermal sample from a skin. In some instances, an adhesive article may have a handling area. In some cases, a handling area may not contact a skin. In some cases, a handling area may not collect an epidermal sample. In some cases, a handling area may not extract an epidermal sample. In some instances, an epidermal sampling area and a handling area may be jointed. In some instances, an epidermal sampling area and a handling area may be separable. In some instances, an epidermal sampling area and a handling area may be jointed and separable. In some cases, an epidermal sampling area may be separated from the handling area by peeling, cutting, tearing or trimming between the epidermal sampling area and handling area.

In some instances, the epidermal sampling kit may comprise a storing envelope. In some cases, a storing envelope may store the adhesive article after the adhesive article has been applied to the skin surface and has extracted an epidermal sample. In some cases, the adhesive article may be stored inside the storing envelope until nucleic acid is extracted from the adhesive article. In some cases, the adhesive article inside the storing envelope may transported from one location to another location. In some cases, the adhesive article may be: (1) applied to the skin area and obtain an epidermal sample, (2) stored inside the storing envelope, and (3) transported from the location where the epidermal sample is obtained to a second location, wherein at the second location, the nucleic acid may be extracted from the epidermal sample.

In some instances, a storing envelope may store at least about 1 adhesive article, about 2 adhesive articles, about 3 adhesive articles, about 4 adhesive articles, about 5 adhesive articles, about 6 adhesive articles, about 7 adhesive articles, about 8 adhesive articles, about 9 adhesive articles, about 10 adhesive articles, about 11 adhesive articles, about 12 adhesive articles, about 13 adhesive articles, about 14 adhesive articles, about 15 adhesive articles, about 16 adhesive articles, about 17 adhesive articles, about 18 adhesive articles, about 19 adhesive articles, about 20 adhesive articles, about 21 adhesive articles, about 22 adhesive articles, about 23 adhesive articles, about 24 adhesive articles, about 25 adhesive articles, about 26 adhesive articles, about 27 adhesive articles, about 28 adhesive articles, about 29 adhesive articles, about 30 adhesive articles, about 31 adhesive articles, about 32 adhesive articles, about 33 adhesive articles, about 34 adhesive articles, about 35 adhesive articles, about 36 adhesive articles, about 37 adhesive articles, about 38 adhesive articles, about 39 adhesive articles, about 40 adhesive articles, about 41 adhesive articles, about 42 adhesive articles, about 43 adhesive articles, about 44 adhesive articles, about 45 adhesive articles, about 46 adhesive articles, about 47 adhesive articles, about 48 adhesive articles, about 49 adhesive articles, about 50 adhesive articles, about 60 adhesive articles, about 70 adhesive articles, about 80 adhesive articles, about 90 adhesive articles, about 100 adhesive articles, or more than 100 adhesive articles.

In some cases, a storing envelope may store at least about 1 adhesive articles. In some cases, a storing envelope may store at least about 2 adhesive articles. In some cases, a storing envelope may store at least about 3 adhesive articles. In some cases, a storing envelope may store at least about 4 adhesive articles. In some cases, a storing envelope may store at least about 5 adhesive articles. In some cases, a storing envelope may store at least about 6 adhesive articles. In some cases, a storing envelope may store at least about 7 adhesive articles. In some cases, a storing envelope may store at least about 8 adhesive articles. In some cases, a storing envelope may store 8 adhesive articles. In some cases, a storing envelope may store at most 8 adhesive articles. In some cases, a storing envelope may store at least about 9 adhesive articles. In some cases, a storing envelope may store at least about 10 adhesive articles. In some cases, a storing envelope may store at least about 11 adhesive articles. In some cases, a storing envelope may store at least about 12 adhesive articles. In some cases, a storing envelope may store at least about 13 adhesive articles. In some cases, a storing envelope may store at least about 14 adhesive articles. In some cases, a storing envelope may store at least about 15 adhesive articles. In some cases, a storing envelope may store at least about 16 adhesive articles. In some cases, a storing envelope may store at least about 17 adhesive articles. In some cases, a storing envelope may store at least about 18 adhesive articles. In some cases, a storing envelope may store at least about 19 adhesive articles. In some cases, a storing envelope may store at least about 20 adhesive articles. In some cases, a storing envelope may store at least about 21 adhesive articles. In some cases, a storing envelope may store at least about 22 adhesive articles. In some cases, a storing envelope may store at least about 23 adhesive articles. In some cases, a storing envelope may store at least about 24 adhesive articles. In some cases, a storing envelope may store at least about 25 adhesive articles. In some cases, a storing envelope may store at least about 26 adhesive articles. In some cases, a storing envelope may store at least about 27 adhesive articles. In some cases, a storing envelope may store at least about 28 adhesive articles. In some cases, a storing envelope may store at least about 29 adhesive articles. In some cases, a storing envelope may store at least about 30 adhesive articles. In some cases, a storing envelope may store at least about 31 adhesive articles. In some cases, a storing envelope may store at least about 32 adhesive articles. In some cases, a storing envelope may store at least about 33 adhesive articles. In some cases, a storing envelope may store at least about 34 adhesive articles. In some cases, a storing envelope may store at least about 35 adhesive articles. In some cases, a storing envelope may store at least about 36 adhesive articles. In some cases, a storing envelope may store at least about 37 adhesive articles. In some cases, a storing envelope may store at least about 38 adhesive articles. In some cases, a storing envelope may store at least about 39 adhesive articles. In some cases, a storing envelope may store at least about 40 adhesive articles. In some cases, a storing envelope may store at least about 41 adhesive articles. In some cases, a storing envelope may store at least about 42 adhesive articles. In some cases, a storing envelope may store at least about 43 adhesive articles. In some cases, a storing envelope may store at least about 44 adhesive articles. In some cases, a storing envelope may store at least about 45 adhesive articles. In some cases, a storing envelope may store at least about 46 adhesive articles. In some cases, a storing envelope may store at least about 47 adhesive articles. In some cases, a storing envelope may store at least about 48 adhesive articles. In some cases, a storing envelope may store at least about 49 adhesive articles. In some cases, a storing envelope may store at least about 50 adhesive articles. In some cases, a storing envelope may store at least about 60 adhesive articles. In some cases, a storing envelope may store at least about 70 adhesive articles. In some cases, a storing envelope may store at least about 80 adhesive articles. In some cases, a storing envelope may store at least about 90 adhesive articles. In some cases, a storing envelope may store at least about 100 adhesive articles.

In some instances, the adhesive article stored in a storing envelope may be stable at a temperature of about −80° C., about −70° C., about −60° C., about −50° C., about −40° C., about −30° C., about −20° C., about −10° C., about 0° C., about 10° C., about 20° C., or about 30° C. In some cases, the adhesive article stored in a storing envelope may be stable at a temperature of about −80° C. In some cases, the adhesive article stored in a storing envelope may be stable at a temperature of about −70° C. In some cases, the adhesive article stored in a storing envelope may be stable at a temperature of about −60° C. In some cases, the adhesive article stored in a storing envelope may be stable at a temperature of about −50° C. In some cases, the adhesive article stored in a storing envelope may be stable at a temperature of about −40° C. In some cases, the adhesive article stored in a storing envelope may be stable at a temperature of about −30° C. In some cases, the adhesive article stored in a storing envelope may be stable at a temperature of about −20° C. In some cases, the adhesive article stored in a storing envelope may be stable at a temperature of about −10° C. In some cases, the adhesive article stored in a storing envelope may be stable at a temperature of about 0° C. In some cases, the adhesive article stored in a storing envelope may be stable at a temperature of about 10° C. In some cases, the adhesive article stored in a storing envelope may be stable at a temperature of about 20° C. In some cases, the adhesive article stored in a storing envelope may be stable at a temperature of about 30° C.

In some cases, the adhesive article stored in a storing envelope may be stable at a temperature from about −80° C. to about 30° C. In some cases, the adhesive article stored in a storing envelope may be stable at a temperature of about −70° C. to about 30° C. In some cases, the adhesive article stored in a storing envelope may be stable at a temperature of about −60° C. to about 30° C. In some cases, the adhesive article stored in a storing envelope may be stable at a temperature of about −50° C. to about 30° C. In some cases, the adhesive article stored in a storing envelope may be stable at a temperature of about −40° C. to about 30° C. In some cases, the adhesive article stored in a storing envelope may be stable at a temperature of about −30° C. to about 30° C. In some cases, the adhesive article stored in a storing envelope may be stable at a temperature of about −20° C. to about 30° C. In some cases, the adhesive article stored in a storing envelope may be stable at a temperature of about −10° C. to about 30° C. In some cases, the adhesive article stored in a storing envelope may be stable at a temperature of about 0° C. to about 30° C. In some cases, the adhesive article stored in a storing envelope may be stable at a temperature of about 4° C. to about 30° C. In some cases, the adhesive article stored in a storing envelope may be stable at a temperature of about 10° C. to about 30° C.

In some instances, the adhesive article stored in a storing envelope may be stable for at least about 1 day, about 2 days, about 3 days, about 4 days, about 5 days, about 6 days, about 7 days, about 2 weeks, about 3 weeks, about 4 weeks, about 2 months, about 3 months, about 4 months, about 5 months, about 6 months, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years, about 11 years, about 12 years, about 13 years, about 14 years, or about 15 years before any cellular materials comprising any nucleic acids of the epidermal sample degrades.

In some instances, the epidermal sampling kit may comprise a liner, a collection container, or a combination thereof. In some cases, the epidermal sampling kit may comprise a collection container. A collection container may comprise a centrifuge tube. In some case, the centrifuge tube may comprise an Eppendorf tube. A collection container may comprise a glass container. A collection container may comprise a plastic container. In some cases, a collection container may have a volume of at least about 5 microliter (μL), 10 μL, 20 μL, 30 μL, 40 μL, 50 μL, 100 μL, 150 μL, 200 μL, 250 μL, 300 μL, 400 μL, 500 μL, 1000 μL, 1.5 milliliter (mL), 2 mL, 5 mL, 10 mL or more. In some cases, a collection container may have a volume of at most about 5 μL, 10 μL, 20 μL, 30 μL, 40 μL, 50 μL, 100 μL, 150 μL, 200 μL, 250 μL, 300 μL, 400 μL, 500 μL, 1000 μL, 1.5 mL, 2 mL, 5 mL, or 10 mL. A collection container may be configured to be sealed. A collection container may not be sealed.

The collection container may comprise an anti-microbial substance. The anti-microbial substance may comprise an anti-bacterial substance, an anti-fungal substance, an anti-parasitic substance, an anti-viral substance, or a combination thereof. The anti-microbial substance may comprise an anti-bacterial substance. The anti-microbial substance may comprise an anti-fungal substance. The anti-microbial substance may comprise an anti-parasitic substance. The anti-microbial substance may comprise an anti-viral substance. The anti-microbial substance may eliminate, kill, or inhibit the growth of a microorganism. The microorganism may comprise a microorganism derived from a skin of a subject. The microorganism may comprise a microorganism not derived from a skin of a subject. For example, the microorganism may be a contaminant not derived from the skin of a subject.

The epidermal sampling kit may comprise a liner. The liner comprise anti-microbial substance. In some cases, a liner may store the adhesive article after the adhesive article has been applied to the skin surface and has extracted an epidermal sample. In some cases, the adhesive article may be stored inside the liner until nucleic acid is extracted from the adhesive article. In some cases, the adhesive article inside the liner may transported from one location to another location. In some cases, a liner may comprise a paper. In other cases, a liner may comprise a material that does not degrade or chemically interact with a substance of an epidermal sample of an adhesive article. In some cases, the adhesive article may be: (1) applied to the skin area and obtain an epidermal sample, (2) stored inside the liner, and (3) transported from the location where the epidermal sample is obtained to a second location, wherein at the second location, the nucleic acid may be extracted from the epidermal sample.

In some instances, storing an adhesive article inside a liner may comprise enclosing the adhesive article with a liner. In some cases, a linear can be folded to enclose an adhesive article. In some cases, a linear can be sealed. In some cases, a linear can be sealed to enclose an adhesive article.

In some instances, a liner may store at least about 1 adhesive article, about 2 adhesive articles, about 3 adhesive articles, about 4 adhesive articles, about 5 adhesive articles, about 6 adhesive articles, about 7 adhesive articles, about 8 adhesive articles, about 9 adhesive articles, about 10 adhesive articles, about 11 adhesive articles, about 12 adhesive articles, about 13 adhesive articles, about 14 adhesive articles, about 15 adhesive articles, about 16 adhesive articles, about 17 adhesive articles, about 18 adhesive articles, about 19 adhesive articles, about 20 adhesive articles, about 21 adhesive articles, about 22 adhesive articles, about 23 adhesive articles, about 24 adhesive articles, about 25 adhesive articles, about 26 adhesive articles, about 27 adhesive articles, about 28 adhesive articles, about 29 adhesive articles, about 30 adhesive articles, about 31 adhesive articles, about 32 adhesive articles, about 33 adhesive articles, about 34 adhesive articles, about 35 adhesive articles, about 36 adhesive articles, about 37 adhesive articles, about 38 adhesive articles, about 39 adhesive articles, about 40 adhesive articles, about 41 adhesive articles, about 42 adhesive articles, about 43 adhesive articles, about 44 adhesive articles, about 45 adhesive articles, about 46 adhesive articles, about 47 adhesive articles, about 48 adhesive articles, about 49 adhesive articles, about 50 adhesive articles, about 60 adhesive articles, about 70 adhesive articles, about 80 adhesive articles, about 90 adhesive articles, about 100 adhesive articles, or more than 100 adhesive articles.

In some cases, a liner may store at least about 1 adhesive articles. In some cases, a liner may store at least about 2 adhesive articles. In some cases, a liner may store at least about 3 adhesive articles. In some cases, a liner may store at least about 4 adhesive articles. In some cases, a liner may store at least about 5 adhesive articles. In some cases, a liner may store at least about 6 adhesive articles. In some cases, a liner may store at least about 7 adhesive articles. In some cases, a liner may store at least about 8 adhesive articles. In some cases, a liner may store 8 adhesive articles. In some cases, a liner may store at most 8 adhesive articles. In some cases, a liner may store at least about 9 adhesive articles. In some cases, a liner may store at least about 10 adhesive articles. In some cases, a liner may store at least about 11 adhesive articles. In some cases, a liner may store at least about 12 adhesive articles. In some cases, a liner may store at least about 13 adhesive articles. In some cases, a liner may store at least about 14 adhesive articles. In some cases, a liner may store at least about 15 adhesive articles. In some cases, a liner may store at least about 16 adhesive articles. In some cases, a liner may store at least about 17 adhesive articles. In some cases, a liner may store at least about 18 adhesive articles. In some cases, a liner may store at least about 19 adhesive articles. In some cases, a liner may store at least about 20 adhesive articles. In some cases, a liner may store at least about 21 adhesive articles. In some cases, a liner may store at least about 22 adhesive articles. In some cases, a liner may store at least about 23 adhesive articles. In some cases, a liner may store at least about 24 adhesive articles. In some cases, a liner may store at least about 25 adhesive articles. In some cases, a liner may store at least about 26 adhesive articles. In some cases, a liner may store at least about 27 adhesive articles. In some cases, a liner may store at least about 28 adhesive articles. In some cases, a liner may store at least about 29 adhesive articles. In some cases, a liner may store at least about 30 adhesive articles. In some cases, a liner may store at least about 31 adhesive articles. In some cases, a liner may store at least about 32 adhesive articles. In some cases, a liner may store at least about 33 adhesive articles. In some cases, a liner may store at least about 34 adhesive articles. In some cases, a liner may store at least about 35 adhesive articles. In some cases, a liner may store at least about 36 adhesive articles. In some cases, a liner may store at least about 37 adhesive articles. In some cases, a liner may store at least about 38 adhesive articles. In some cases, a liner may store at least about 39 adhesive articles. In some cases, a liner may store at least about 40 adhesive articles. In some cases, a liner may store at least about 41 adhesive articles. In some cases, a liner may store at least about 42 adhesive articles. In some cases, a liner may store at least about 43 adhesive articles. In some cases, a liner may store at least about 44 adhesive articles. In some cases, a liner may store at least about 45 adhesive articles. In some cases, a liner may store at least about 46 adhesive articles. In some cases, a liner may store at least about 47 adhesive articles. In some cases, a liner may store at least about 48 adhesive articles. In some cases, a liner may store at least about 49 adhesive articles. In some cases, a liner may store at least about 50 adhesive articles. In some cases, a liner may store at least about 60 adhesive articles. In some cases, a liner may store at least about 70 adhesive articles. In some cases, a liner may store at least about 80 adhesive articles. In some cases, a liner may store at least about 90 adhesive articles. In some cases, a liner may store at least about 100 adhesive articles.

In some instances, the adhesive article stored in a liner may be stable at a temperature of about −80° C., about −70° C., about −60° C., about −50° C., about −40° C., about −30° C., about −20° C., about −10° C., about 0° C., about 10° C., about 20° C., or about 30° C. In some cases, the adhesive article stored in a liner may be stable at a temperature of about −80° C. In some cases, the adhesive article stored in a liner may be stable at a temperature of about −70° C. In some cases, the adhesive article stored in a liner may be stable at a temperature of about −60° C. In some cases, the adhesive article stored in a liner may be stable at a temperature of about −50° C. In some cases, the adhesive article stored in a liner may be stable at a temperature of about −40° C. In some cases, the adhesive article stored in a liner may be stable at a temperature of about −30° C. In some cases, the adhesive article stored in a liner may be stable at a temperature of about −20° C. In some cases, the adhesive article stored in a liner may be stable at a temperature of about −10° C. In some cases, the adhesive article stored in a liner may be stable at a temperature of about 0° C. In some cases, the adhesive article stored in a liner may be stable at a temperature of about 10° C. In some cases, the adhesive article stored in a liner may be stable at a temperature of about 20° C. In some cases, the adhesive article stored in a liner may be stable at a temperature of about 30° C.

In some cases, the adhesive article stored in a liner may be stable at a temperature from about −80° C. to about 30° C. In some cases, the adhesive article stored in a liner may be stable at a temperature of about −70° C. to about 30° C. In some cases, the adhesive article stored in a liner may be stable at a temperature of about −60° C. to about 30° C. In some cases, the adhesive article stored in a liner may be stable at a temperature of about −50° C. to about 30° C. In some cases, the adhesive article stored in a liner may be stable at a temperature of about −40° C. to about 30° C. In some cases, the adhesive article stored in a liner may be stable at a temperature of about −30° C. to about 30° C. In some cases, the adhesive article stored in a liner may be stable at a temperature of about −20° C. to about 30° C. In some cases, the adhesive article stored in a liner may be stable at a temperature of about −10° C. to about 30° C. In some cases, the adhesive article stored in a liner may be stable at a temperature of about 0° C. to about 30° C. In some cases, the adhesive article stored in a liner may be stable at a temperature of about 4° C. to about 30° C. In some cases, the adhesive article stored in a liner may be stable at a temperature of about 10° C. to about 30° C.

In some instances, the adhesive article stored in a liner may be stable for at least about 1 day, about 2 days, about 3 days, about 4 days, about 5 days, about 6 days, about 7 days, about 2 weeks, about 3 weeks, about 4 weeks, about 2 months, about 3 months, about 4 months, about 5 months, about 6 months, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years, about 11 years, about 12 years, about 13 years, about 14 years, or about 15 years before any cellular materials comprising any nucleic acids of the epidermal sample degrades.

In some cases, epidermal sampling with an epidermal sampling kit may comprise (1) applying an adhesive article to a skin, (2) removing the adhesive article that comprises an epidermal sample, (3) storing the adhesive article inside a storing envelope. In some cases, epidermal sampling with an epidermal sampling kit may comprise (1) applying an adhesive article to a skin, (2) removing the adhesive article that comprises an epidermal sample, (3) enclosing the adhesive article with a liner, (4) storing the adhesive article enclosed by the liner inside a storing envelope.

In some instance, an epidermal sampling kit may comprise at least one glove. In some cases, an epidermal sampling kit may comprise at least two gloves. In some cases, an epidermal sampling kit may comprise at least three gloves. In some cases, an epidermal sampling kit may comprise at least four gloves. In some cases, an epidermal sampling kit may comprise at least five gloves. In some instance, an epidermal sampling kit may comprise at least one, at least two, at least three, at least four, at least five or more gloves. In some cases, a glove is worn by an individual that applies an adhesive article on a skin of a subject. In some cases, a glove is worn by an individual that detaches, removes, or strips an adhesive article off a skin of a subject. In some cases, a glove is worn by an individual that handles an adhesive article comprising an epidermal sample of a subject.

### Nucleic Acid

In some instances, nucleic acids derived from epidermal samples may comprise deoxyribonucleic acid (DNA), ribonucleic acid (RNA), a variant thereof, or a combination thereof. In some cases, nucleic acids derived from epidermal samples may comprise DNA. In some cases, nucleic acids derived from epidermal samples may comprise RNA. In some cases, nucleic acids derived from epidermal samples may comprise DNA and RNA. In some cases, DNA derived from epidermal samples may comprise single-stranded DNA or double-stranded DNA. In some cases, DNA derived from epidermal samples may comprise single-stranded DNA. In some cases, DNA derived from epidermal samples may comprise double-stranded DNA. In some cases, DNA derived from epidermal samples may comprise complementary DNA (cDNA).

In some cases, RNA derived from epidermal samples may comprise single-stranded or double-stranded RNA. In some cases, RNA derived from epidermal samples may comprise single-stranded RNA. In some cases, RNA derived from epidermal samples may comprise double-stranded RNA. In some instances, RNA may comprise a messenger RNA (mRNA).

Nucleic acid may be pure or substantially pure after nucleic acid extraction. Nucleic acid may need to be purified after extraction to become pure or substantially pure. A pure or substantially pure nucleic acid in a sample may comprise at least about 50%, 60%, 70%, 80%, 85%, 90%, 95%, 99% or greater amount (mole, weight, or volume) of nucleic acid in the sample. A pure or substantially pure nucleic acid in a sample may also comprise less than about 40%, 30%, 20%, 15%, 14%, 13%, 12%, 11%, 10%, 9%, 8%, 7%, 6%, 5%, 4%, 3%, 2%, 1% or less amount (mole, weight, or volume) of the nucleic acid present in the sample.

A species of nucleic acid may have a specific sequence or be chemically defined nucleic acid.

In some instances, nucleic acid derived from epidermal samples may be processed, treated, or converted from nucleic acids extracted from epidermal samples. In some cases, conversions of nucleic acids may comprise an amplification reaction, a reverse transcription reaction, a primer extension reaction, a ligation reaction, or a combination thereof. In some cases, conversions of nucleic acids may comprise an amplification reaction. In some cases, conversions of nucleic acids may comprise a reverse transcription reaction. In some cases, conversions of nucleic acids may comprise a primer extension reaction. In some cases, conversions of nucleic acids may comprise a ligation reaction.

In some instances, an amplification reaction may comprise an asymmetric amplification reaction, a helicase-dependent amplification (HDA), a ligase chain reaction (LCR), a loop mediated isothermal amplification (LAMP), a multiple displacement amplification (MDA), a nucleic acid sequence based amplification (NASBA), a polymerase chain reaction (PCR), a primer extension, a recombinase polymerase amplification (RPA), a rolling circle amplification (RCA), a self-sustained sequence replication (3SR), or a strand displacement amplification (SDA). In some cases, an amplification reaction may comprise an asymmetric amplification reaction. In some cases, an amplification reaction may comprise a helicase-dependent amplification (HDA). In some cases, an amplification reaction may comprise a ligase chain reaction (LCR). In some cases, an amplification reaction may comprise a loop mediated isothermal amplification (LAMP). In some cases, an amplification reaction may comprise a multiple displacement amplification (MDA). In some cases, an amplification reaction may comprise a nucleic acid sequence based amplification (NASBA). In some cases, an amplification reaction may comprise a polymerase chain reaction (PCR). In some cases, an amplification reaction may comprise a primer extension. In some cases, an amplification reaction may comprise a recombinase polymerase amplification (RPA). In some cases, an amplification reaction may comprise a rolling circle amplification (RCA). In some cases, an amplification reaction may comprise a self-sustained sequence replication (3SR). In some cases, an amplification reaction may comprise a strand displacement amplification (SDA). In some cases, an amplification reaction may comprise the derivatives or combinations of any amplifications thereof.

In some instances, a reverse transcription reaction may convert an RNA to a DNA. In some cases, a reverse transcription reaction may convert an RNA to a complementary DNA (cDNA). In some cases, an RNA may be converted to a cDNA using a reverse transcriptase. A reverse transcription (RT), in some instance, may comprise priming an RNA template with an oligonucleotide. An example of an oligonucleotide may comprise an RT primer. In some cases, an RT primer may comprise DNA. In some cases, an RT primer may comprise a single-stranded sequence of deoxythymine, a primer with a random nucleotide sequence, or a gene-specific primer with a gene-specific nucleotide sequence. In one case, an RT primer may be single-stranded. Once primed with an RT primer, a reverse transcriptase may synthesize a DNA sequence using the complementarity of the RNA template nucleotide sequence. In some cases, a RT may be a linear synthesis reaction. In other cases, a RT may be a non-linear synthesis reaction.

In some cases, conversions of nucleic acids may comprise a ligation reaction. In some cases, a ligation reaction may ligate a nucleotide molecule/sequence to an RNA or a DNA. In some cases, a nucleotide molecule/sequence may comprise a nucleic acid adaptor. In other cases, a nucleotide molecule/sequence may comprise an identifiable nucleotide sequence. One such identifiable nucleotide sequence may comprise a barcode sequence. A nucleic acid adaptor or a barcode sequence may be used in for assaying, identifying, or sequencing nucleic acids.

In some cases, nucleic acids extracted from epidermal samples may comprise deoxyribonucleic acid (DNA), ribonucleic acid (RNA), or a combination thereof. In some cases, nucleic acids extracted from epidermal samples may comprise DNA. In some cases, nucleic acids extracted from epidermal samples may comprise RNA. In some cases, nucleic acids extracted from epidermal samples may comprise DNA and RNA. In some cases, DNA extracted from epidermal samples may comprise single-stranded DNA or double-stranded DNA. In some cases, DNA derived from epidermal samples may comprise single-stranded DNA or double-stranded DNA. In some cases, DNA extracted from epidermal samples may comprise single-stranded DNA. In some cases, DNA extracted from epidermal samples may comprise double-stranded DNA. In some cases, RNA extracted from epidermal samples may comprise single-stranded or double-stranded RNA. In some cases, RNA extracted from epidermal samples may comprise single-stranded RNA. In some cases, RNA extracted from epidermal samples may comprise double-stranded RNA. In some cases, the nucleic acid, DNA, or RNA extracted from an epidermal sample may be cell-free.

In some instances, a nucleic acid may comprise coding or non-coding sequence. In some cases, a nucleic acid may comprise coding sequence. In some cases, a nucleic acid may comprise non-coding sequence. In some cases, a nucleic acid may comprise coding and non-coding sequence.

In some instances, a set of nucleic acid sequences comprises a transcriptome, a genome, or a combination thereof.

### Genome

A genome may comprise a human genome. In some cases, identifying a genome may comprise identifying the sequences of the genome. In other cases, identifying a genome may comprise identifying the mutation, variation, and/or abundance in the sequence of the genome relative to a reference genome.

In some instances, a genome may comprise a substantial portion of the cellular DNA that comprises genetic information. In some cases, a genome may comprise at least about 50%, 60%, 70%, 75%, 80%, 85%, 90%, 91%, 92%, 93%, 94%, 95%, 96%, 97%, 98%, 99%, or 100% of the cellular DNA that comprises genetic information. In some cases, a genome may comprise at least about 50% of the cellular DNA that comprises genetic information. In some cases, a genome may comprise at least about 60% of the cellular DNA that comprises genetic information. In some cases, a genome may comprise at least about 70% of the cellular DNA that comprises genetic information. In some cases, a genome may comprise at least about 75% of the cellular DNA that comprises genetic information. In some cases, a genome may comprise at least about 80% of the cellular DNA that comprises genetic information. In some cases, a genome may comprise at least about 85% of the cellular DNA that comprises genetic information. In some cases, a genome may comprise at least about 90% of the cellular DNA that comprises genetic information. In some cases, a genome may comprise at least about 91% of the cellular DNA that comprises genetic information. In some cases, a genome may comprise at least about 92% of the cellular DNA that comprises genetic information. In some cases, a genome may comprise at least about 93% of the cellular DNA that comprises genetic information. In some cases, a genome may comprise at least about 94% of the cellular DNA that comprises genetic information. In some cases, a genome may comprise at least about 95% of the cellular DNA that comprises genetic information. In some cases, a genome may comprise at least about 96% of the cellular DNA that comprises genetic information. In some cases, a genome may comprise at least about 97% of the cellular DNA that comprises genetic information. In some cases, a genome may comprise at least about 98% of the cellular DNA that comprises genetic information. In some cases, a genome may comprise at least about 99% of the cellular DNA that comprises genetic information. In some cases, a genome may comprise about 100% of the cellular DNA that comprises genetic information. In some cases, genomic DNA may not be chemically modified. In other cases, genomic DNA may be chemically modified. Chemical modification of DNA, in some cases, may comprise any methylation or epigenetic modification described in this disclosure.

### Transcriptome

In some instances, a transcriptome may comprise a substantial portion of the cellular transcripts. In some cases, a transcriptome may comprise at least about 50%, 60%, 70%, 75%, 80%, 85%, 90%, 91%, 92%, 93%, 94%, 95%, 96%, 97%, 98%, 99%, or 100% of the cellular transcripts. In some cases, a transcriptome may comprise at least about 50% of the cellular transcripts. In some cases, a transcriptome may comprise at least about 60% of the cellular transcripts. In some cases, a transcriptome may comprise at least about 70% of the cellular transcripts. In some cases, a transcriptome may comprise at least about 75% of the cellular transcripts. In some cases, a transcriptome may comprise at least about 80% of the cellular transcripts. In some cases, a transcriptome may comprise at least about 85% of the cellular transcripts. In some cases, a transcriptome may comprise at least about 90% of the cellular transcripts. In some cases, a transcriptome may comprise at least about 91% of the cellular transcripts. In some cases, a transcriptome may comprise at least about 92% of the cellular transcripts. In some cases, a transcriptome may comprise at least about 93% of the cellular transcripts. In some cases, a transcriptome may comprise at least about 94% of the cellular transcripts. In some cases, a transcriptome may comprise at least about 95% of the cellular transcripts. In some cases, a transcriptome may comprise at least about 96% of the cellular transcripts. In some cases, a transcriptome may comprise at least about 97% of the cellular transcripts. In some cases, a transcriptome may comprise at least about 98% of the cellular transcripts. In some cases, a transcriptome may comprise at least about 99% of the cellular transcripts. In some cases, a transcriptome may comprise about 100% of the cellular transcripts.

In some instances, a transcriptome may comprise a substantial portion of the cellular mRNAs. In some cases, a transcriptome may comprise at least about 50%, 60%, 70%, 75%, 80%, 85%, 90%, 91%, 92%, 93%, 94%, 95%, 96%, 97%, 98%, 99%, or 100% of the cellular mRNAs. In some cases, a transcriptome may comprise at least about 50% of the cellular mRNAs. In some cases, a transcriptome may comprise at least about 60% of the cellular mRNAs. In some cases, a transcriptome may comprise at least about 70% of the cellular mRNAs. In some cases, a transcriptome may comprise at least about 75% of the cellular mRNAs. In some cases, a transcriptome may comprise at least about 80% of the cellular mRNAs. In some cases, a transcriptome may comprise at least about 85% of the cellular mRNAs. In some cases, a transcriptome may comprise at least about 90% of the cellular mRNAs. In some cases, a transcriptome may comprise at least about 91% of the cellular mRNAs. In some cases, a transcriptome may comprise at least about 92% of the cellular mRNAs. In some cases, a transcriptome may comprise at least about 93% of the cellular mRNAs. In some cases, a transcriptome may comprise at least about 94% of the cellular mRNAs. In some cases, a transcriptome may comprise at least about 95% of the cellular mRNAs. In some cases, a transcriptome may comprise at least about 96% of the cellular mRNAs. In some cases, a transcriptome may comprise at least about 97% of the cellular mRNAs. In some cases, a transcriptome may comprise at least about 98% of the cellular mRNAs. In some cases, a transcriptome may comprise at least about 99% of the cellular mRNAs. In some cases, a transcriptome may comprise about 100% of the cellular mRNAs.

In some instances, a transcriptome may comprise a substantial portion of the cellular mRNA, tRNA, rRNA, or noncoding RNA molecules. In some cases, a transcriptome may comprise at least about 50%, 60%, 70%, 75%, 80%, 85%, 90%, 91%, 92%, 93%, 94%, 95%, 96%, 97%, 98%, 99%, or 100% of the cellular mRNA, tRNA, rRNA, or noncoding RNA molecules. In some cases, a transcriptome may comprise at least about 50% of the cellular mRNA, tRNA, rRNA, or noncoding RNA molecules. In some cases, a transcriptome may comprise at least about 60% of the cellular mRNA, tRNA, rRNA, or noncoding RNA molecules. In some cases, a transcriptome may comprise at least about 70% of the cellular mRNA, tRNA, rRNA, or noncoding RNA molecules. In some cases, a transcriptome may comprise at least about 75% of the cellular mRNA, tRNA, rRNA, or noncoding RNA molecules. In some cases, a transcriptome may comprise at least about 80% of the cellular mRNA, tRNA, rRNA, or noncoding RNA molecules. In some cases, a transcriptome may comprise at least about 85% of the cellular mRNA, tRNA, rRNA, or noncoding RNA molecules. In some cases, a transcriptome may comprise at least about 90% of the cellular mRNA, tRNA, rRNA, or noncoding RNA molecules. In some cases, a transcriptome may comprise at least about 91% of the cellular mRNA, tRNA, rRNA, or noncoding RNA molecules. In some cases, a transcriptome may comprise at least about 92% of the cellular mRNA, tRNA, rRNA, or noncoding RNA molecules. In some cases, a transcriptome may comprise at least about 93% of the cellular mRNA, tRNA, rRNA, or noncoding RNA molecules. In some cases, a transcriptome may comprise at least about 94% of the cellular mRNA, tRNA, rRNA, or noncoding RNA molecules. In some cases, a transcriptome may comprise at least about 95% of the cellular mRNA, tRNA, rRNA, or noncoding RNA molecules. In some cases, a transcriptome may comprise at least about 96% of the cellular mRNA, tRNA, rRNA, or noncoding RNA molecules. In some cases, a transcriptome may comprise at least about 97% of the cellular mRNA, tRNA, rRNA, or noncoding RNA molecules. In some cases, a transcriptome may comprise at least about 98% of the cellular mRNA, RNA, rRNA, or noncoding RNA molecules. In some cases, a transcriptome may comprise at least about 99% of the cellular mRNA, tRNA, rRNA, or noncoding RNA molecules. In some cases, a transcriptome may comprise about 100% of the cellular mRNA, tRNA, rRNA, or noncoding RNA molecules.

### Epigenetic Regulation Epigenome

Epigenetic may comprise DNA modification, histone modification, and/or regulation by non-coding RNAs. The term “epigenetic mark” may comprise the site, sequence, location of a genetic material that is under the regulation or modification by epigenetic. For example, an epigenetic mark may comprise methylated DNA or nucleotide comprising cytosine (C). In some case, the methylated C may be followed by a guanine (G).

In some instances, epigenetic regulation may comprise DNA modification, histone modification, or non-coding RNA. In some cases, epigenetic regulation may comprise DNA modification. In some instances, DNA may be modified. In some case, a DNA modification may comprise a chemical modification at a base of the DNA. In some cases, DNA modification may comprise DNA methylation. In some cases, cytosine in the DNA may be methylated. In some cases, methylated DNA may occur in a cytosine followed by a guanine (i.e., CpG site). A CpG site may comprise be comprise 5′-C-phosphate-G-3′. A CpG site on a genome may be the cytosine and guanine appearing consecutively on the same strand of nucleic acid (the p may comprise the phosphodiester bond joining the two nucleotides). In some cases, when CpG sites occur frequently in a region of a genome, the region may be referred to as a CpG island. The region may appear frequently at the start of promoters in eukaryotes and generally can indicate the presence of a coding region. In some cases, a sequence to be methylated may be CG-rich. A CG-rich sequence may comprise at least about 50%, 60%, 70%, 75%, 80%, 85%, 90%, 91%, 92%, 93%, 94%, 95%, 96%, 97%, 98%, 99%, or 100% of nucleotides that are either a C or a G. In some cases, a sequence comprising a CG dinucleotide an at least an extra nucleotide. The extra nucleotide may comprise a C or a G. In other cases, the extra nucleotide may also comprise an A or a T. In some cases, a CpG site may comprise about 1, 2, 3, 4, 5, 6, 7, 8, 9, 10, 20, 30, 40, 50, 60, 70, 80, 90, 100 or more nucleotides. In some cases, a CpG site may comprise about 1, 2, 3, 4, 5, 6, 7, 8, 9, 10, 20, 30, 40, 50, 60, 70, 80, 90, 100 or more C or G nucleotides. In some cases, cytosine of DNA may be modified. In some cases, cytosine of DNA may be modified by a methylation. In some cases, a methylated cytosine (5mC) may be further modified. In some cases, a 5mC may be reverted to an unmethylated cytosine. In some cases, a 5mC may be modified by oxidation. In some cases, a 5mC may be oxidized to a 5-hydroxymethylcytosine (5hmC). In some cases, a 5hmC may be oxidized to a 5-formylcytosine (5fC). In some cases, a 5fC may be oxidized to a 5-carboxylcytosine (5caC). In some cases, a modified cytosine may comprise a 5mC, a 5hmC, a 5fC, a 5caC, or any combinations thereof. In other cases, an adenine of DNA may be methylated, i.e., N⁶-methyladenine (6mA).

In some instances, an epigenome may comprise a substantial portion of the DNA that has undergone epigenetic modification. In some cases, an epigenome may comprise at least about 50%, 60%, 70%, 75%, 80%, 85%, 90%, 91%, 92%, 93%, 94%, 95%, 96%, 97%, 98%, 99%, or 100% of the DNA that are epigenetically modified. In some cases, an epigenome may comprise at least about 50% of the DNA that are epigenetically modified. In some cases, an epigenome may comprise at least about 60% of the DNA that are epigenetically modified. In some cases, an epigenome may comprise at least about 70% of the DNA that are epigenetically modified. In some cases, an epigenome may comprise at least about 75% of the DNA that are epigenetically modified. In some cases, an epigenome may comprise at least about 80% of the DNA that are epigenetically modified. In some cases, an epigenome may comprise at least about 85% of the DNA that are epigenetically modified. In some cases, an epigenome may comprise at least about 90% of the DNA that are epigenetically modified. In some cases, an epigenome may comprise at least about 91% of the DNA that are epigenetically modified. In some cases, an epigenome may comprise at least about 92% of the DNA that are epigenetically modified. In some cases, an epigenome may comprise at least about 93% of the DNA that are epigenetically modified. In some cases, an epigenome may comprise at least about 94% of the DNA that are epigenetically modified. In some cases, an epigenome may comprise at least about 95% of the DNA that are epigenetically modified. In some cases, an epigenome may comprise at least about 96% of the DNA that are epigenetically modified. In some cases, an epigenome may comprise at least about 97% of the DNA that are epigenetically modified. In some cases, an epigenome may comprise at least about 98% of the DNA that are epigenetically modified. In some cases, an epigenome may comprise at least about 99% of the DNA that are epigenetically modified. In some cases, an epigenome may comprise about 100% of the DNA that are epigenetically modified.

In some instances, a methylome may comprise a substantial portion of the DNA that has undergone methylation. In some cases, a methylome may comprise at least about 50%, 60%, 70%, 75%, 80%, 85%, 90%, 91%, 92%, 93%, 94%, 95%, 96%, 97%, 98%, 99%, or 100% of the DNA that are methylated. In some cases, a methylome may comprise at least about 50% of the DNA that are methylated. In some cases, a methylome may comprise at least about 60% of the DNA that are methylated. In some cases, a methylome may comprise at least about 70% of the DNA that are methylated. In some cases, a methylome may comprise at least about 75% of the DNA that are methylated. In some cases, a methylome may comprise at least about 80% of the DNA that are methylated. In some cases, a methylome may comprise at least about 85% of the DNA that are methylated. In some cases, a methylome may comprise at least about 90% of the DNA that are methylated. In some cases, a methylome may comprise at least about 91% of the DNA that are methylated. In some cases, a methylome may comprise at least about 92% of the DNA that are methylated. In some cases, a methylome may comprise at least about 93% of the DNA that are methylated. In some cases, a methylome may comprise at least about 94% of the DNA that are methylated. In some cases, a methylome may comprise at least about 95% of the DNA that are methylated. In some cases, a methylome may comprise at least about 96% of the DNA that are methylated. In some cases, a methylome may comprise at least about 97% of the DNA that are methylated. In some cases, a methylome may comprise at least about 98% of the DNA that are methylated. In some cases, a methylome may comprise at least about 99% of the DNA that are methylated. In some cases, a methylome may comprise about 100% of the DNA that are methylated.

### Metagenome

When referring to the skin or epidermis, metagenome refers to a complete set or a substantial portion of a complete set of microorganisms present the skin or epidermis, respectively. Metagenome may also refer to microbiome.

In some instances, a metagenome of a subject may comprise genomic DNA from a source different from the subject. In some instances, a source different from a subject comprise a species different from the subject. In some cases, the species different from the subject may comprise a bacterium, a virus, an archaeon, a fungus, a protozoon, or a combination thereof. In some cases, the species different from the subject may comprise at least two of a bacterium, a virus, an archaeon, a fungus, or a protozoon. In some cases, the species different from the subject may comprise at least three of a bacterium, a virus, an archaeon, a fungus, or a protozoon. In some cases, the species different from the subject may comprise at least four of a bacterium, a virus, an archaeon, a fungus, or a protozoon. In some cases, the species different from the subject may comprise a bacterium. In some cases, the species different from the subject may comprise a virus. In some cases, the species different from the subject may comprise an archaeon. In some cases, the species different from the subject may comprise a fungus. In some cases, the species different from the subject may comprise a protozoon.

In some instances, a metagenome may comprise at least about 1×10\hat{ }1 different numbers of species of organisms. In some cases, a metagenome may comprise at least about 1×10\hat{ }2 different numbers of species of organisms. In some cases, a metagenome may comprise at least about 1×10\hat{ }3 different numbers of species of organisms. In some cases, a metagenome may comprise at least about 1×10\hat{ }4 different numbers of species of organisms. In some cases, a metagenome may comprise at least about 1×10\hat{ }5 different numbers of species of organisms. In some cases, a metagenome may comprise at least about 1×10\hat{ }6 different numbers of species of organisms. In some cases, a metagenome may comprise at least about 1×10\hat{ }7 different numbers of species of organisms. In some cases, a metagenome may comprise at least about 1×10\hat{ }8 different numbers of species of organisms. In some cases, a metagenome may comprise at least about 1×10\hat{ }9 different numbers of species of organisms. In some cases, a metagenome may comprise at least about 1×10\hat{ }10 different numbers of species of organisms. In some cases, a metagenome may comprise at least about 1×10\hat{ }11 different numbers of species of organisms. In some cases, a metagenome may comprise at least about 1×10\hat{ }12 different numbers of species of organisms. In some cases, a metagenome may comprise at least about 1×10\hat{ }13 different numbers of species of organisms. In some cases, a metagenome may comprise at least about 1×10\hat{ }14 different numbers of species of organisms. In some cases, a metagenome may comprise at least about 1×10\hat{ }15 different numbers of species of organisms. In some cases, a metagenome may comprise at least about 1×10\hat{ }16 different numbers of species of organisms. In some cases, a metagenome may comprise at least about 1×10\hat{ }17 different numbers of species of organisms. In some cases, a metagenome may comprise at least about 1×10\hat{ }18 different numbers of species of organisms. In some cases, a metagenome may comprise at least about 1×10\hat{ }19 different numbers of species of organisms. In some cases, a metagenome may comprise at least about 1×10\hat{ }20 different numbers of species of organisms.

In some instances, a metagenome may comprise at least about 1×10\hat{ }1 different numbers of organisms. In some cases, a metagenome may comprise at least about 1×10\hat{ }2 different numbers of organisms. In some cases, a metagenome may comprise at least about 1×10\hat{ }5 different numbers of organisms. In some cases, a metagenome may comprise at least about 1×10\hat{ }10 different numbers of organisms. In some cases, a metagenome may comprise at least about 1×10\hat{ }20 different numbers of organisms. In some cases, a metagenome may comprise at least about 1×10\hat{ }50 different numbers of organisms. In some cases, a metagenome may comprise at least about 1×10\hat{ }100 different numbers of organisms. In some cases, a metagenome may comprise at least about 1×10\hat{ }200 different numbers of organisms. In some cases, a metagenome may comprise at least about 1×10\hat{ }500 different numbers of organisms. In some cases, a metagenome may comprise at least about 1×10\hat{ }1000 different numbers of organisms. In some cases, a metagenome may comprise at least about 1×10\hat{ }2000 different numbers of organisms. In some cases, a metagenome may comprise at least about 1×10\hat{ }5000 different numbers of organisms. In some cases, a metagenome may comprise at least about 1×10\hat{ }10000 different numbers of organisms. In some cases, a metagenome may comprise at least about 1×10\hat{ }20000 different numbers of organisms. In some cases, a metagenome may comprise at least about 1×10\hat{ }50000 different numbers of organisms.

In some instances, a metagenome may comprise at least about 5×10\hat{ }1 different numbers of genes In some cases, a metagenome may comprise at least about 5×10\hat{ }2 different numbers of genes In some cases, a metagenome may comprise at least about 5×10\hat{ }3 different numbers of genes In some cases, a metagenome may comprise at least about 5×10\hat{ }4 different numbers of genes In some cases, a metagenome may comprise at least about 5×10\hat{ }5 different numbers of genes In some cases, a metagenome may comprise at least about 5×10\hat{ }6 different numbers of genes In some cases, a metagenome may comprise at least about 5×10\hat{ }7 different numbers of genes In some cases, a metagenome may comprise at least about 5×10\hat{ }8 different numbers of genes In some cases, a metagenome may comprise at least about 5×10\hat{ }9 different numbers of genes In some cases, a metagenome may comprise at least about 5×10\hat{ }10 different numbers of genes.

In some instances, a metagenome may comprise at least about 0.2 kilograms of nucleic acids. In some cases, a metagenome may comprise at least about 0.4 kilograms of nucleic acids. In some cases, a metagenome may comprise at least about 0.6 kilograms of nucleic acids. In some cases, a metagenome may comprise at least about 0.8 kilograms of nucleic acids. In some cases, a metagenome may comprise at least about 1 kilograms of nucleic acids. In some cases, a metagenome may comprise at least about 1.2 kilograms of nucleic acids. In some cases, a metagenome may comprise at least about 1.4 kilograms of nucleic acids. In some cases, a metagenome may comprise at least about 1.6 kilograms of nucleic acids. In some cases, a metagenome may comprise at least about 1.8 kilograms of nucleic acids. In some cases, a metagenome may comprise at least about 2 kilograms of nucleic acids. In some cases, a metagenome may comprise at least about 2.2 kilograms of nucleic acids. In some cases, a metagenome may comprise at least about 2.4 kilograms of nucleic acids. In some cases, a metagenome may comprise at least about 2.6 kilograms of nucleic acids. In some cases, a metagenome may comprise at least about 2.8 kilograms of nucleic acids. In some cases, a metagenome may comprise at least about 3 kilograms of nucleic acids. In some cases, a metagenome may comprise at least about 3.2 kilograms of nucleic acids. In some cases, a metagenome may comprise at least about 3.4 kilograms of nucleic acids. In some cases, a metagenome may comprise at least about 3.6 kilograms of nucleic acids. In some cases, a metagenome may comprise at least about 3.8 kilograms of nucleic acids. In some cases, a metagenome may comprise at least about 4 kilograms of nucleic acids.

In some instances, a metagenome of a subject may comprise a substantial portion of the genomic DNA that is different source/species the subject. In some cases, a metagenome of a subject may comprise at least about 50%, 60%, 70%, 75%, 80%, 85%, 90%, 91%, 92%, 93%, 94%, 95%, 96%, 97%, 98%, 99%, or 100% of the genomic DNA that is different source/species the subject. In some cases, a metagenome of a subject may comprise at least about 50% of the genomic DNA that is different source/species the subject. In some cases, a metagenome of a subject may comprise at least about 60% of the genomic DNA that is different source/species the subject. In some cases, a metagenome of a subject may comprise at least about 70% of the genomic DNA that is different source/species the subject. In some cases, a metagenome of a subject may comprise at least about 75% of the genomic DNA that is different source/species the subject. In some cases, a metagenome of a subject may comprise at least about 80% of the genomic DNA that is different source/species the subject. In some cases, a metagenome of a subject may comprise at least about 85% of the genomic DNA that is different source/species the subject. In some cases, a metagenome of a subject may comprise at least about 90% of the genomic DNA that is different source/species the subject. In some cases, a metagenome of a subject may comprise at least about 91% of the genomic DNA that is different source/species the subject. In some cases, a metagenome of a subject may comprise at least about 92% of the genomic DNA that is different source/species the subject. In some cases, a metagenome of a subject may comprise at least about 93% of the genomic DNA that is different source/species the subject. In some cases, a metagenome of a subject may comprise at least about 94% of the genomic DNA that is different source/species the subject. In some cases, a metagenome of a subject may comprise at least about 95% of the genomic DNA that is different source/species the subject. In some cases, a metagenome of a subject may comprise at least about 96% of the genomic DNA that is different source/species the subject. In some cases, a metagenome of a subject may comprise at least about 97% of the genomic DNA that is different source/species the subject. In some cases, a metagenome of a subject may comprise at least about 98% of the genomic DNA that is different source/species the subject. In some cases, a metagenome of a subject may comprise at least about 99% of the genomic DNA that is different source/species the subject. In some cases, a metagenome of a subject may comprise about 100% of the genomic DNA that is different source/species the subject.

### Nucleic Acid Extraction

In some instances, nucleic acid may be extracted from an epidermal sample. In some cases, DNA or RNA may be extracted from an epidermal sample. In some cases, DNA may be extracted from an epidermal sample. In some cases, RNA may be extracted from an epidermal sample. In some cases, DNA and RNA may be extracted from an epidermal sample. In some instances, a nucleic acid may be extracted from lysed cells or cellular materials in an epidermal sample.

In some instances, extracting nucleic acid may comprise using a solid support. In some cases, a solid support for extracting nucleic acid may comprise a bead. Such a bead, in some cases, may comprise a magnetic bead. In some cases, a magnetic bead may comprise a particle with superparamagnetic properties. In the presence of an external magnetic field, the magnetic bead may exhibit magnetic behaviors, forming clumps or aggregates with each other, separating from other non-magnetic molecules. In some cases, the magnetic beads may be configured to bind any nucleic acid molecules described herein. In some cases, the magnetic beads may bind DNA or RNA. In some cases, the magnetic beads may bind DNA. In some cases, the magnetic beads may bind RNA. In some cases, the magnetic beads may bind DNA and RNA. In some cases, the magnetic beads may bind nucleic acid, DNA, or RNA via a nucleic acid binding moiety. In some cases, a nucleic acid binding moiety that can bind nucleic acid may comprise an oligonucleotide, glass, silica, latex, or a polymeric material. In some instances, a bead may be coated by a material. In some cases, a bead may be coated by silica. In some cases, a bead may comprise silica-coated magnetic beads. In some a bead may have a diameter of about at least about 0.1 micrometer (μm), at least about 0.2 μm, at least about 0.3 μm, at least about 0.4 μm, at least about 0.5 μm, at least about 0.6 μm, at least about 0.7 μm, at least about 0.8 μm, at least about 0.9 μm, at least about 1 μm, at least about 1.5 μm, at least about 2 μm, at least about 2.5 μm, at least about 3 μm, at least about 3.5 μm, at least about 4 μm, at least about 4.5 μm, at least about 5 μm, at least about 5.5 μm, at least about 6 μm, at least about 6.5 μm, at least about 7 μm, at least about 7.5 μm, at least about 8 μm, at least about 8.5 μm, at least about 9 μm, at least about 9.5 μm, at least about 10 μm, or more than 10 μm.

In some instances, extracting a nucleic acid from an epidermal sample may comprise alcohol precipitation using ethanol, methanol or isopropyl alcohol. In some cases, extracting a nucleic acid from an epidermal sample may comprise phenol, chloroform, or any combination thereof. In some cases, extracting a nucleic acid from an epidermal sample may comprise cesium chloride. In some cases, extracting a nucleic acid from an epidermal sample may comprise sodium, potassium or ammonium acetate or any other salt.

In some instances, extracting a nucleic acid from an epidermal sample may also comprise kits comprising RNeasy™ (Qiagen, Valencia, Calif.) or TriReagent™ (Molecular Research Center, Inc, Cincinnati, Ohio).

In some instances, a nucleic acid may be stored in water, Tris buffer, or Tris-EDTA buffer after extraction from an epidermal sample. In some cases, a nucleic acid may be stored at a temperature of about −100° C., about −80° C., about −50° C., about −40° C., about −30° C., about −20° C., about −10° C., about −5° C., about −4° C., about −3° C., about −2° C., about −1° C., about 0° C., about 1° C., about 2° C., about 3° C., about 4° C., about 5° C., about 10° C., about 15° C., about 20° C., about 25° C., about 30° C., or higher than about 30° C. In some cases, a nucleic acid may be stored for less than about 1 day, at least about 1 day, at least about 2 days, at least about 3 days, at least about 4 days, at least about 5 days, at least about 6 days, at least about 7 days, at least about 2 weeks, at least about 3 weeks, at least about 4 weeks, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 2 years, at least about 3 years, at least about 4 years, at least about 5 years, at least about 6 years, at least about 7 years, at least about 8 years, at least about 9 years, at least about 10 years, at least about 11 years, at least about 12 years, at least about 13 years, at least about 14 years, at least about 15 years, at least about 16 years, at least about 17 years, at least about 18 years, at least about 19 years, or at least about 20 years.

### Sequencing

In some cases, assaying a nucleic acid may comprise sequencing the nucleic acid or any derivative thereof. In other cases, assaying a nucleic acid may comprise identifying the nucleic acid or the derivative thereof. In other cases, assaying a nucleic acid may comprise identifying the sequence of the nucleic acid or the derivative thereof. In some cases, identifying the sequence of the nucleic acid or the derivative thereof may comprise identifying the mutation or variation of the nucleic acid or the derivative thereof, relative to the wildtype sequence of the nucleic acid. In some cases, assaying a nucleic acid may comprise identifying the modification of the nucleic acid sequence (e.g., an epigenetic modification). In other cases, assaying a nucleic acid may comprise identifying the sequence of the nucleic acid or the derivative thereof. In other cases, assaying a nucleic acid may comprise identifying the expression level of the nucleic acid or the derivative thereof. In some cases, assaying or identifying a nucleic acid may also comprise sequencing the nucleic acid or a derivative thereof.

In some instances, sequencing may comprise whole-genome sequencing. In some cases, sequencing may comprise whole-genome methylation sequencing. In some cases, sequencing may comprise whole-genome sequencing or whole-genome methylation sequencing. In some cases, sequencing may comprise whole-genome sequencing and whole-genome methylation sequencing. In some cases, sequencing may comprise next-generation sequencing. In some cases, sequencing may comprise 2nd generation sequencing. In some cases, sequencing may comprise 3^{rd} generation sequencing. In some cases, sequencing may comprise 4^{th} generation sequencing.

In some instances, sequencing may comprise chain termination sequencing, high-throughput sequencing, mass spectrophotometry sequencing, massively parallel signature sequencing, Maxam-Gilbert sequencing, nanopore sequencing, primer walking, pyrosequencing, Sanger sequencing, semiconductor sequencing, sequencing-by-hybridization, sequencing-by-ligation, sequencing-by-synthesis, single-molecule sequencing, shotgun sequencing, bisulfite sequencing, or any combination thereof. In some cases, sequencing may comprise chain termination sequencing. In some cases, sequencing may comprise high-throughput sequencing. In some cases, sequencing may comprise mass spectrophotometry sequencing. In some cases, sequencing may comprise massively parallel signature sequencing. In some cases, sequencing may comprise Maxam-Gilbert sequencing. In some cases, sequencing may comprise nanopore sequencing. In some cases, sequencing may comprise primer walking. In some cases, sequencing may comprise pyrosequencing. In some cases, sequencing may comprise Sanger sequencing. In some cases, sequencing may comprise semiconductor sequencing. In some cases, sequencing may comprise sequencing-by-hybridization. In some cases, sequencing may comprise sequencing-by-ligation. In some cases, sequencing may comprise sequencing-by-synthesis. In some cases, sequencing may comprise single-molecule sequencing. In some cases, sequencing may comprise shotgun sequencing.

In some instances, identifying a nucleic acid may comprise sequencing, a PCR, a microarray analysis, or fluorescent hybridization. In some cases, identifying a nucleic acid may comprise a PCR. In some cases, identifying a nucleic acid may comprise a microarray analysis. In some cases, identifying a nucleic acid may comprise fluorescent hybridization. In some cases, a PCR may comprise an allele-specific PCR, an assembly PCR, an asymmetric PCR, a co-amplification at lower denaturation temperature-PCR, a dial-out PCR, a digital PCR, an emulsion PCR, a gene-specific PCR, a helicase-dependent PCR, a hot start PCR, an inverse PCR, a Klenow-based PCR, a ligation-mediated PCR, a methylation-specific PCR, a miniprimer PCR, a multiplex PCR, a nested PCR, a nested PCR, an overlap-extension PCR, a quantitative PCR, a real-time PCR, a thermal asymmetric interlaced PCR and touchdown PCR, a touchdown PCR, or a two-tailed PCR.

### Enzymatic Conversion for Methylated Nucleic Acid Sequencing

In some instances, sequencing may identify a methylated nucleotide of a nucleic acid. In some cases, sequencing that can identify a methylated nucleotide of a nucleic acid may comprise Enzymatic Methyl sequencing. In some cases, sequencing that can identify a methylated nucleotide of a nucleic acid may processing a nucleic acid with at least one enzyme that can modify a base of a nucleotide of the nucleic acid. In some cases, the enzyme may convert a base of a nucleotide to another base. In some cases, the enzyme may convert a hydroxylation of 5-methylcytosine (5mC) to a 5-hydroxymethylcytosine (5hmC). In some cases, the enzyme may convert a 5hmC to a 5-formylcytosine (5fC). In some cases, an enzyme may convert a 5fC to a 5-carboxycytosine (5caC). In some cases, a 5hmC may also be converted to a 5(beta-glucosyloxymethyl) cytosine (5gmC). In some cases, a cytosine may be converted to an uracil. In some cases, a 5mC may be converted to a thymine (T). In some cases, a 5hmC may be converted to a 5-Hydroxymethyluracil (5hmU).

In some instances, an enzyme that can modify a base or convert a base to a different base may comprise a family of methylcytosine dioxygenase enzymes, an enzyme of a uridine diphosphate (UDP)-glucose: deoxyribonucleic acid (DNA) (UDP-glucose:DNA) beta-D-glucosyltransferase family, an enzyme of a deaminase family, or a combination thereof. In some cases, an enzyme that can modify a base or convert a base to a different base may comprise at least two of a family of methylcytosine dioxygenase enzymes, an enzyme of a UDP-glucose:DNA beta-D-glucosyltransferase family, or an enzyme of a deaminase family. In some cases, an enzyme that can modify a base or convert a base to a different base may comprise an enzyme of a family of methylcytosine dioxygenase enzymes, an enzyme of a UDP-glucose:DNA beta-D-glucosyltransferase family, and an enzyme of a deaminase family.

In some instances, an enzyme that can modify a base or convert a base to a different base may comprise an enzyme of a Ten-Eleven Translocation (TET) family, an enzyme of a UDP-glucose:DNA beta-D-glucosyltransferase family, an enzyme of a deaminase family, or a combination thereof. In some cases, an enzyme that can modify a base or convert a base to a different base may comprise at least two of an enzyme of a TET family, an enzyme of a UDP-glucose:DNA beta-D-glucosyltransferase family, or an enzyme of a deaminase family. In some cases, an enzyme that can modify a base or convert a base to a different base may comprise an enzyme of a TET family, an enzyme of a UDP-glucose:DNA beta-D-glucosyltransferase family, and an enzyme of a deaminase family.

In some instances, methylcytosine dioxygenase enzymes may methylate cytosine. In some cases, methylcytosine dioxygenase enzymes may comprise the family of ten-eleven translocation (TET) family. In some cases, TET enzymes may catalyze the hydroxylation of 5-methylcytosine (5mC) to 5-hydroxymethylcytosine (5hmC). In some cases, TET enzymes may also catalyze the oxidation of 5hmC to 5-formylcytosine (5fC). In some cases, TET enzymes may also catalyze the oxidation of 5fC to 5-carboxycytosine (5caC).

In some cases, the enzyme of the TET family may comprise TET1, TET2, TET3, or a functional equivalent derivative thereof. In some cases, the enzyme of the TET family may comprise at least two of TET1, TET2, or TET3; or any derivatives thereof. In some cases, the enzyme of the TET family may comprise TET1, TET2, and TET3; or any derivatives thereof. In some cases, the enzyme of the TET family may comprise TET1, or a functional equivalent derivative thereof. In some cases, the enzyme of the TET family may comprise TET2 or a functional equivalent derivative thereof. In some cases, the enzyme of the TET family may comprise TET3 or a functional equivalent derivative thereof.

The functional test may be context-dependent and may depend on the identity of the protein or polypeptide. In some cases, the functional test may comprise an enzymatic activity or a chemical-binding activity.

In some instances, UDP-glucose:DNA beta-D-glucosyltransferase may comprise a class of enzyme that catalyzes the transfer of a beta-D-glucosyl residue from UDP-glucose to a glucosylhydroxymethylcytosine residue in DNA. In some cases, an UDP-glucose:DNA beta-D-glucosyltransferase may convert a 5-hydroxymethylcytosine (5hmC) to a 5(beta-glucosyloxymethyl) cytosine (5gmC).

In some cases, the enzyme of a UDP-glucose:DNA beta-D-glucosyltransferase family may be from a bacterium, or the derivative thereof. In some cases, the enzyme of a UDP-glucose:DNA beta-D-glucosyltransferase family may be from a microorganism, or the derivative thereof. In some cases, the enzyme of a UDP-glucose:DNA beta-D-glucosyltransferase family may be from E. coli; or the derivative thereof.

In some instances, a deaminase may comprise a cytosine deaminase. In some cases, a deaminase may comprise an adenine deaminase. In some instances, a deaminase may also comprise a guanine deaminase. In some cases, deamination of a nucleotide may convert a nucleotide to a different nucleotide. For example, deamination may convert a cytosine to an uracil, a 5-methylcytosine (5hmC) to a thymine, a guanine to a xanthine, an adenine (A) to a hypoxanthine. In some cases, both DNA and RNA may be deaminated.

In some cases, an enzyme of the deaminase family may comprise Apolipoprotein B mRNA Editing Catalytic Polypeptide-like 1 (APOBEC1), Apolipoprotein B mRNA Editing Catalytic Polypeptide-like 3A (APOBEC3A), Apolipoprotein B mRNA Editing Catalytic Polypeptide-like 3B (APOBEC3B), Apolipoprotein B mRNA Editing Catalytic Polypeptide-like 3C (APOBEC3C), Apolipoprotein B mRNA Editing Catalytic Polypeptide-like 3D (APOBEC3D), Apolipoprotein B mRNA Editing Catalytic Polypeptide-like 3E (APOBEC3E), Apolipoprotein B mRNA Editing Catalytic Polypeptide-like 3F (APOBEC3F), Apolipoprotein B mRNA Editing Catalytic Polypeptide-like 3H (APOBEC3H), Apolipoprotein B mRNA Editing Catalytic Polypeptide-like 3G (APOBEC3G), affects HIV Activation-induced cytidine deaminase (AICDA), Cytidine deaminase (CDA), dCMP deaminase (DCTD), or a functional equivalent derivative thereof. In some cases, an enzyme of the deaminase family may comprise APOBEC1 or a functional derivative thereof. In some cases, an enzyme of the deaminase family may comprise APOBEC3A or a functional derivative thereof. In some cases, an enzyme of the deaminase family may comprise APOBEC3B or a functional derivative thereof. In some cases, an enzyme of the deaminase family may comprise APOBEC3C or a functional derivative thereof. In some cases, an enzyme of the deaminase family may comprise APOBEC3D or a functional derivative thereof. In some cases, an enzyme of the deaminase family may comprise APOBEC3E or a functional derivative thereof. In some cases, an enzyme of the deaminase family may comprise APOBEC3F or a functional derivative thereof. In some cases, an enzyme of the deaminase family may comprise APOBEC3H or a functional derivative thereof. In some cases, an enzyme of the deaminase family may comprise APOBEC3G or a functional derivative thereof. In some cases, an enzyme of the deaminase family may comprise affects HIV Activation-induced cytidine deaminase (AICDA) or a functional derivative thereof. In some cases, an enzyme of the deaminase family may comprise Cytidine deaminase (CDA) or a functional derivative thereof. In some cases, an enzyme of the deaminase family may comprise dCMP deaminase (DCTD) or a functional derivative thereof.

In some instances, enzymes that can modify a base or convert a base to a different base may comprise TET2 or a functional equivalent derivative thereof; the UDP-glucose:DNA beta-D-glucosyltransferase from E. coli or a functional equivalent derivative thereof; or an APOBEC3A or a functional equivalent derivative thereof. In some instances, enzymes that can modify a base or convert a base to a different base may comprise at least two of TET2 or a functional equivalent derivative thereof; the UDP-glucose:DNA beta-D-glucosyltransferase from E. coli or a functional equivalent derivative thereof; or an APOBEC3A or a functional equivalent derivative thereof. In some instances, enzymes that can modify a base or convert a base to a different base may comprise TET2 or a functional equivalent derivative thereof; the UDP-glucose:DNA beta-D-glucosyltransferase from E. coli or a functional equivalent derivative thereof; and an APOBEC3A or a functional equivalent derivative thereof.

In some instances, sequencing that can identify a methylated nucleotide of a nucleic acid may comprise sequencing the modified nucleic acid or a derivative thereof and unmodified nucleic acid or a derivative thereof and comparing the sequence reads. In some cases, Enzymatic Methyl sequencing may comprise (1) modifying a nucleic acid with enzymes that can modify a base or convert a base to a different base described thereof and (2) sequencing the modified nucleic acid or a derivative thereof and unmodified version of the nucleic acid or a derivative thereof and comparing the sequence reads. In some cases, a methylated C may be read as a C in the modified nucleic thereof. In some cases, a unmethylated C may be read as a T in the modified nucleic thereof. In some cases, a methylated C may be read as a C in the modified nucleic thereof, and a unmethylated C may be read as a T in the modified nucleic thereof.

### Enzymatic and Chemical Conversion for Methylated Nucleic Acid Sequencing

In some instances, sequencing that can identify a methylated nucleotide of a nucleic acid may comprise TET-assisted pyridine borane sequencing (TAPS) sequencing. In some cases, sequencing that can identify a methylated nucleotide of a nucleic acid may comprise processing a nucleic acid with at least one enzyme that can modify a base of a nucleotide of the nucleic acid. In some cases, the enzyme may convert a base of a nucleotide to another base. In some cases, the enzyme may convert a 5mC to a 5hmC. In some cases, the enzyme may convert a 5hmC to a 5fC. In some cases, an enzyme may convert a 5fC to a 5caC. In some instances, an enzyme that can modify a base or convert a base to a different base may comprise a family of methylcytosine dioxygenase enzymes. In some instances, methylcytosine dioxygenase enzymes may methylate cytosine. In some instances, an enzyme that can modify a base or convert a base to a different base may comprise an enzyme from the TET family. In some cases, methylcytosine dioxygenase enzymes may comprise the TET family. In some cases, an enzyme of the TET family may comprise any enzymes from the TET family described elsewhere in this disclosure.

In some instances, sequencing that can identify a methylated nucleotide of a nucleic acid may comprise a chemical reaction of a nucleotide. In some cases, sequencing that can identify a methylated nucleotide of a nucleic acid may comprise a reduction reaction of a nucleotide. In some cases, sequencing that can identify a methylated nucleotide of a nucleic acid may comprise a reduction reaction of a nucleotide using a chemical. In some cases, a chemical used in sequencing that can identify a methylated nucleotide of a nucleic acid may comprise pyridine borane or a derivative thereof. In some cases, a chemical used in sequencing that can identify a methylated nucleotide of a nucleic acid may comprise pyridine borane. In some cases, a chemical used in sequencing that can identify a methylated nucleotide of a nucleic acid may comprise a derivative of pyridine borane.

In some instances, sequencing that can identify a methylated nucleotide of a nucleic acid may comprise reducing a 5caC to a dihydrouracil (DHU). In some cases, a chemical may reduce a 5caC to a DHU. In some cases, pyridine borane or a derivative thereof may reduce a 5caC to a DHU. In some cases, pyridine borane may reduce a 5caC to a DHU. In some cases, a derivative of pyridine borane or a derivative thereof may reduce a 5caC to a DHU.

In some instances, sequencing that can identify a methylated nucleotide of a nucleic acid may comprise an amplification reaction. In some cases, sequencing that can identify a methylated nucleotide of a nucleic acid may comprise a PCR. In some cases, an amplification reaction may modify a base of a nucleotide. In some cases, a PCR may modify a base of a nucleotide. In some cases, an amplification reaction may convert a base of a nucleotide to another base. In some cases, a PCR may convert a base of a nucleotide to another base. In some cases, an amplification reaction may convert a DHU to a T. In some cases, a PCR may convert a DHU to a T.

In some instances, TAPS sequencing may comprise (1) modifying a nucleic acid with an enzyme that can modify a base or convert a base to a different base described thereof; (2) modifying the nucleic acid subsequent to (1) with a chemical that can modify a base or convert a base to a different base described thereof; (3) subject the modified nucleic acid subsequent to (1) and (2) an amplification reaction described thereof; (4) sequencing the modified nucleic acid or a derivative thereof and unmodified version of the nucleic acid or a derivative thereof and comparing the sequence reads. In some cases, a methylated C may be read as a T in the modified nucleic thereof. In some cases, a unmethylated C may be read as a C in the modified nucleic thereof. In some cases, a methylated C may be read as a T in the modified nucleic thereof, and a unmethylated C may be read as a C in the modified nucleic thereof.

### Epidermal Properties

In some instances, an epidermal property may comprise intrinsic epidermal age, extrinsic epidermal age, epidermal firmness and elasticity, moisture, regeneration capacity, antioxidant protection capacity, inflammatory control, extent of epidermal infection, extent of epidermal disease condition, extent of epidermal pigmentation, extent of ultraviolet (UV) exposure, extent of UV damage, or a combination thereof. In some cases, an epidermal property may comprise intrinsic epidermal age. In some cases, an epidermal property may comprise extrinsic epidermal age. In some cases, an epidermal property may comprise epidermal firmness and elasticity. In some cases, an epidermal property may comprise moisture. In some cases, an epidermal property may comprise regeneration capacity. In some cases, an epidermal property may comprise antioxidant protection capacity. In some cases, an epidermal property may comprise inflammatory control. In some cases, an epidermal property may comprise extent of epidermal infection. In some cases, an epidermal property may comprise extent of epidermal disease condition. In some cases, an epidermal property may comprise extent of epidermal pigmentation. In some cases, an epidermal property may comprise extent of UV exposure. In some cases, an epidermal property may comprise extent of UV damage.

In some instances, an epidermal property may comprise the presence or absence of a skin disease. In some cases, an epidermal property may comprise the risk or probability of developing or suffering from a skin disease. In some cases, a skin disease may comprise a bruise, a sebaceous cyst, a skin tag, actinic keratoses, allergic contact dermatitis, allergic purpura, an allergic reaction, an infection, basal cell carcinoma, dermatitis, Dermatitis herpetiformis, erythema multiforme, erythema nodosum, hidradenitis suppurative, hives, irritant contact dermatitis, Kaposi's sarcoma, keloid, lipoma, lupus erythematosus, melanoma, Pityriasis rosea, psoriasis, seborrhea, squamous cell carcinoma, xanthelasma, or any combination thereof.

In some instances, an epidermal property may be identified or determined by assaying an epidermal sample. In some cases, an epidermal property may be identified or determined by assaying the nucleic acid of the epidermal sample. In some cases, an epidermal property may be identified or determined by assaying the genetic materials, genomes, transcripts, transcriptomes, epigenetic marks, epigenomes, methylated DNAs, methylomes, microorganisms, microbiomes, metagenomes, or a combination thereof of a skin sample. In some cases, because different genetic materials, genomes, transcripts, transcriptomes, epigenetic marks, epigenomes, methylated DNAs, methylomes, microorganisms, microbiome, metagenome, or any combination thereof may affect the gene expression profile of a skin cell, different epidermal properties may have different or unique signatures of the genetic materials, genomes, transcripts, transcriptomes, epigenetic marks, epigenomes, methylated DNAs, methylomes, microorganisms, microbiomes, metagenomes, or a combination. Different genetic materials, genomes, transcripts, transcriptomes, epigenetic marks, epigenomes, methylated DNAs, methylomes, microorganisms, microbiome, metagenome, or any combination thereof may identify a different/unique skin property thereof. In some instance, determining an epidermal property of an epidermal sample may comprise analyzing the nucleic acids of the epidermal sample. In some cases, determining an epidermal property of an epidermal sample may comprise analyzing the sequences of the nucleic acids of the epidermal sample. In some cases, analyzing the sequences of the nucleic acids of the epidermal sample may comprise determining the expression level or sequences of the nucleic acids of the epidermal sample. In some cases, analyzing the sequences of the nucleic acids of the epidermal sample may comprise determining the methylation level or methylated sequences of the nucleic acids of the epidermal sample. In some cases, analyzing the sequences of the nucleic acids of the epidermal sample may comprise determining the level or sequences of the microbial nucleic acids of the epidermal sample.

In some cases, an epidermal property may be represented by the expression level of a nucleic acid or a portion thereof. In some cases, a difference in the epidermal properties of two epidermal samples may be represented by a difference in the expression levels of a nucleic acid or a portion thereof in the two epidermal samples. The difference in the expression levels of the nucleic acid or a portion thereof in the two epidermal samples can reflect a difference of at least about 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, or 100% between the epidermal properties of the two epidermal samples. The difference in the expression levels of the nucleic acid or a portion thereof in the two epidermal samples can reflect a difference of at most about 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, or 100% between the epidermal properties of the two epidermal samples.

In some cases, an epidermal property may be represented by the methylation level of a nucleic acid or a portion thereof. In some cases, a difference in the epidermal properties of two epidermal samples may be represented by a difference in the methylation level of a nucleic acid or a portion thereof in the two epidermal samples. The difference of the methylation levels of the nucleic acid or a portion thereof in the two epidermal samples can reflect a difference of at least about 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, or 100% between the epidermal properties of the two epidermal samples. The difference of the methylation levels of the nucleic acid or a portion thereof in the two epidermal samples can reflect a difference of at most about 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, or 100% between the epidermal properties of the two epidermal samples.

A nucleic acid or a portion thereof for assaying the epidermal property may comprise any one of sites described in Table 11 below.

TABLE 11
Selected Chromosomal Locations

| | Chromosomal locations |
|---|---|
| NO | (GRCh38 human reference genome) |
| 1 | chr1:3268173-3268175 |
| 2 | chr1:3268184-3268186 |
| 3 | chr1:3268194-3268196 |
| 4 | chr1:3268200-3268202 |
| 5 | chr1:3268208-3268210 |
| 6 | chr1:3268210-3268212 |
| 7 | chr1:3268226-3268228 |
| 8 | chr1:114099884-114099886 |
| 9 | chr1:114099966-114099968 |
| 10 | chr1:244163961-244163963 |
| 11 | chr10:1725623-1725625 |
| 12 | chr10:1725681-1725683 |
| 13 | chr10:1725687-1725689 |
| 14 | chr10:2714614-2714616 |
| 15 | chr10:2734649-2734651 |
| 16 | chr10:2734797-2734799 |
| 17 | chr10:2734828-2734830 |
| 18 | chr10:2734838-2734840 |
| 19 | chr10:2734848-2734850 |
| 20 | chr10:2734850-2734852 |
| 21 | chr10:2734872-2734874 |
| 22 | chr10:2734911-2734913 |
| 23 | chr10:2734939-2734941 |
| 24 | chr10:2734983-2734985 |
| 25 | chr10:99233398-99233400 |
| 26 | chr10:129884230-129884232 |
| 27 | chr10:129884234-129884236 |
| 28 | chr10:132810171-132810173 |
| 29 | chr10:132810180-132810182 |
| 30 | chr10:132810187-132810189 |
| 31 | chr10:132810189-132810191 |
| 32 | chr10:132981858-132981860 |
| 33 | chr10:132981876-132981878 |
| 34 | chr10:132981878-132981880 |
| 35 | chr10:132981918-132981920 |
| 36 | chr11:59426361-59426363 |
| 37 | chr12:7711479-7711481 |
| 38 | chr13:35476505-35476507 |
| 39 | chr14:29389951-29389953 |
| 40 | chr14:29390108-29390110 |
| 41 | chr14:95072037-95072039 |
| 42 | chr15:96471117-96471119 |
| 43 | chr16:1026234-1026236 |
| 44 | chr16:51149561-51149563 |
| 45 | chr16:55091619-55091621 |
| 46 | chr16:76194435-76194437 |
| 47 | chr16:85956123-85956125 |
| 48 | chr16:86878972-86878974 |
| 49 | chr16:87647581-87647583 |
| 50 | chr16:89159342-89159344 |
| 51 | chr16:89159344-89159346 |
| 52 | chr17:74351946-74351948 |
| 53 | chr17:79210458-79210460 |
| 54 | chr19:53851971-53851973 |
| 55 | chr19:53851999-53852001 |
| 56 | chr2:52880703-52880705 |
| 57 | chr2:63052293-63052295 |
| 58 | chr2:63052946-63052948 |
| 59 | chr2:63052961-63052963 |
| 60 | chr2:63052964-63052966 |
| 61 | chr2:63053028-63053030 |
| 62 | chr2:63053240-63053242 |
| 63 | chr2:66579003-66579005 |
| 64 | chr2:66583491-66583493 |
| 65 | chr2:181694454-181694456 |
| 66 | chr2:181694456-181694458 |
| 67 | chr2:181694458-181694460 |
| 68 | chr20:20289457-20289459 |
| 69 | chr21:39336863-39336865 |
| 70 | chr21:39336880-39336882 |
| 71 | chr21:39336885-39336887 |
| 72 | chr5:2889714-2889716 |
| 73 | chr5:2890142-2890144 |
| 74 | chr5:2890153-2890155 |
| 75 | chr5:2890161-2890163 |
| 76 | chr5:52149859-52149861 |
| 77 | chr5:76953253-76953255 |
| 78 | chr6:129947530-129947532 |
| 79 | chr6:164099707-164099709 |
| 80 | chr6:164099736-164099738 |
| 81 | chr7:1256270-1256272 |
| 82 | chr7:2932700-2932702 |
| 83 | chr7:2932707-2932709 |
| 84 | chr7:2932725-2932727 |
| 85 | chr7:2932741-2932743 |
| 86 | chr7:2932757-2932759 |
| 87 | chr7:25854237-25854239 |
| 88 | chr7:82805692-82805694 |
| 89 | chr7:101498873-101498875 |
| 90 | chr7:101498875-101498877 |
| 91 | chr7:157541203-157541205 |
| 92 | chr7:157541274-157541276 |
| 93 | chr8:1688112-1688114 |
| 94 | chr8:98949263-98949265 |
| 95 | chr9:77016708-77016710 |
| 96 | chr9:77016710-77016712 |
| 97 | chr9:77016726-77016728 |
| 98 | chr9:77016964-77016966 |
| 99 | chr9:77017922-77017924 |
| 100 | chr9:80564795-80564797 |

A site defined in Table 11 may comprise a CpG site and a 5′ upstream sequence, a 3′ downstream sequence, a complement thereof, or a combination thereof. In an illustrative example, the site defined in chr1: 3268173-3268175 of Table 11 may comprise a CpG site located between the 3268173^{rd} and 3268176^{th} nucleotides on Chromosome 1 of a GRCh38 human reference genome. Such definition may also comprise the 5′ upstream sequence (any sequence upstream or 5′ sequence of the 3268173^{rd} nucleotide on Chromosome 1 of the GRCh38 human reference genome), 3′ downstream sequence (any sequence downstream or 3′ sequence of the 3268175^{h} nucleotide on Chromosome 1 of the GRCh38 human reference genome), a complement thereof, or a combination thereof of the CpG site located at chr1: 3268173-3268175. In some cases, when defining a site for assaying the epidermal property of an epidermal sample, a 5′ upstream sequence of the site may comprise at least about 1, 2, 3, 4, 5, 6, 7, 8, 9, 10, 20, 30, 40, 50, 60, 70, 80, 90, 100, 500, 1000 or more nucleotides 5′ of the site. In some cases, when defining a site for assaying the epidermal property of an epidermal sample, a 3′ downstream sequence of the site may comprise at least about 1, 2, 3, 4, 5, 6, 7, 8, 9, 10, 20, 30, 40, 50, 60, 70, 80, 90, 100, 500, 1000 or more nucleotides 3′ of the site. In some cases, a site for assaying the epidermal property of an epidermal sample may comprise a functional equivalent of the site. The functional equivalent of a site in a reference genome may comprise a comparable site in another reference genome. In some cases, a site and the functional equivalent thereof (or the comparable site) may share at least about 30%, 40%, 50%, 60%, 70%, 75%, 80%, 85%, 90%, 91%, 92%, 93%, 94%, 95%, 96%, 97%, 98%, 99%, or 100% sequence identity. In some cases, a site and the functional equivalent thereof (or the comparable site) may share at least about 30%, 40%, 50%, 60%, 70%, 75%, 80%, 85%, 90%, 91%, 92%, 93%, 94%, 95%, 96%, 97%, 98%, 99%, or 100% sequence identity in their 5′ upstream sequence, 3′ downstream sequence, or a combination thereof. Similar definitions may apply to other sites defined in Table 11, or any sequences or sites used for assaying the epidermal property of an epidermal sample.

In some cases, analyzing a sequence of the nucleic acid of the epidermal sample may comprise enriching the sequence of the nucleic acid from a plurality of nucleic acids. The enriching may comprise amplifying the sequence of the nucleic acid from the plurality of nucleic acids. Such amplifying may comprise any amplification reaction described elsewhere in this disclosure. The enriching may also comprise fluorescent hybridization using a probe that is specific to the sequence of the nucleic acid being enriched. The sequence of the nucleic acid being enriched may comprise any one of the sites in Table 11. The sequence of the nucleic acid being enriched can also comprise any other sites or chromosomal locations of a reference genome. The reference genome may comprise a GRCh38 human reference genome. The reference genome may comprise a microbial genome or a microbiome. A site may be selected for determining the epidermal property may using the methods described in EXAMPLE 5 or elsewhere in this disclosure.

An epidermal property may be represented by the methylation patterns of a plurality of nucleic acids. In some cases, a difference in the epidermal properties of two epidermal samples may be represented by a difference in the methylation patterns of a plurality of nucleic acids of the two epidermal samples. The difference of the methylation patterns may comprise a plurality of nucleic acids that exhibit differences in the methylation levels. The difference of the methylation patterns of a plurality of nucleic acids in the two epidermal samples can reflect a difference of at least about 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, or 100% between the epidermal properties of the two epidermal samples. The difference of the methylation patterns of the nucleic acid or a portion thereof in the two epidermal samples can reflect a difference of at least about 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, or 100% between the epidermal properties of the two epidermal samples.

In some cases, an epidermal property may be represented by the expression level of a microbial nucleic acid or a portion thereof. In some cases, a difference in the epidermal properties of two epidermal samples may be represented by a difference in the expression levels of a microbial nucleic acid or a portion thereof in the two epidermal samples. The difference of the expression levels of the microbial nucleic acid or a portion thereof in the two epidermal samples can reflect a difference of at least about 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, or 100% between the epidermal properties of the two epidermal samples. The difference of the expression levels of the microbial nucleic acid or a portion thereof in the two epidermal samples can reflect a difference of at most about 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, or 100% between the epidermal properties of the two epidermal samples.

An epidermal property may be represented by the metagenome patterns of a plurality of microbial nucleic acids. In some cases, a difference in the epidermal properties of two epidermal samples may be represented by a difference in metagenome patterns of a plurality of microbial nucleic acids of the two epidermal samples. The difference of the metagenome patterns may comprise a number of microbial nucleic acids that exhibits a difference in the expression level. The difference in the metagenome patterns of a plurality of microbial nucleic acids of the two epidermal samples can reflect a difference of at least about 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, or 100% between the epidermal properties of the two epidermal samples. The difference in the patterns of the microbial nucleic acid or a portion thereof of the two epidermal samples t can reflect a difference of at least about 1%, 2%, 3%, 4%, 5%, 6%, 7%, 8%, 9%, 10%, 15%, 20%, 30%, 40%, 50%, 60%, 70%, 75%, 80%, 85%, 90%, 91%, 92%, 93%, 94%, 95%, 96%, 97%, 98%, 99%, or 100% between the epidermal properties of the two epidermal samples.

Other sites may also be used to determine the epidermal property. The site can comprise any other sites or chromosomal locations of a reference genome. The reference genome may comprise GRCh38 human reference genome. The reference genome may comprise a microbial genome or a microbiome. A site may be selected for determining the epidermal property by using the methods described in EXAMPLE 5 or elsewhere in this disclosure.

In some instances, analyzing the sequences of the nucleic acids of the epidermal sample may comprise subjecting the sequences, genetic materials, genomes, transcripts, transcriptomes, epigenetic marks, epigenomes, methylated DNAs, methylomes, microorganisms, microbiomes, metagenomes, or any combination thereof to a trained algorithm. In some cases, a trained algorithm may be trained using reference data. In some cases, the reference data may comprise the genetic materials, genomes, transcripts, transcriptomes, epigenetic marks, epigenomes, methylated DNAs, methylomes, microorganisms, microbiomes, metagenomes, or any combination thereof in reference samples that have known epidermal properties. In some cases, the trained algorithm may associate any genetic materials, genomes, transcripts, transcriptomes, epigenetic marks, epigenomes, methylated DNAs, methylomes, microorganisms, microbiomes, metagenomes, or any combination thereof to any specific epidermal properties. When presented with input variables comprising the sequences, genetic materials, genomes, transcripts, transcriptomes, epigenetic marks, epigenomes, methylated DNAs, methylomes, microorganisms, microbiomes, metagenomes, or any combination thereof of the epidermal sample form a subject, the algorithm may associate any of the input variables to any of epidermal properties.

In some instances, an algorithm for determining an epidermal property may comprise a supervised learning algorithm, a semi-supervised learning algorithm, or a non-supervised learning algorithm. In some cases, an algorithm for determining an epidermal property may comprise at least two of supervised learning algorithm, a semi-supervised learning algorithm, or a non-supervised learning algorithm. In some cases, an algorithm for determining an epidermal property may comprise a supervised learning algorithm. In some cases, an algorithm for determining an epidermal property may comprise a semi-supervised learning algorithm. In some cases, an algorithm for determining an epidermal property may comprise a non-supervised learning algorithm.

In some instances, an algorithm for determining an epidermal property may comprise artificial neural network, Bayes classifier, blind source separation, decision tree, eigenmatrices, Gaussian radical basis function, joint-approximate diagonalization, kernel and polynomial kernel analysis, linear and nonlinear independent component analysis (ICA), natural gradient maximum likelihood estimation, non-Gaussianity analysis, principal component analysis (PCA), linear regression model, sequential floating forward selection, support vector machine, or a combination thereof. In some instances, an algorithm for determining an epidermal property may comprise artificial neural network. In some cases, an algorithm for determining an epidermal property may comprise Bayes classifier. In some cases, an algorithm for determining an epidermal property may comprise blind source separation. In some cases, an algorithm for determining an epidermal property may comprise decision tree. In some cases, an algorithm for determining an epidermal property may comprise eigenmatrices. In some cases, an algorithm for determining an epidermal property may comprise Gaussian radical basis function. In some cases, an algorithm for determining an epidermal property may comprise joint-approximate diagonalization. In some cases, an algorithm for determining an epidermal property may comprise kernel and polynomial kernel analysis. In some cases, an algorithm for determining an epidermal property may comprise linear and nonlinear independent component analysis (ICA). In some cases, an algorithm for determining an epidermal property may comprise natural gradient maximum likelihood estimation. In some cases, an algorithm for determining an epidermal property may comprise non-Gaussianity analysis. In some cases, an algorithm for determining an epidermal property may comprise principal component analysis (PCA). In some cases, an algorithm for determining an epidermal property may comprise a regression model. A regression model may comprise a linear regression model or a non-linear regression model. A regression model may comprise a linear regression model. A regression model may comprise a non-linear regression model. In some cases, an algorithm for determining an epidermal property may comprise sequential floating forward selection. In some cases, an algorithm for determining an epidermal property may comprise support vector machine.

In some cases, an algorithm for determining an epidermal property may comprise at least two of artificial neural network, Bayes classifier, blind source separation, decision tree, eigenmatrices, Gaussian radical basis function, joint-approximate diagonalization, kernel and polynomial kernel analysis, linear and nonlinear independent component analysis (ICA), natural gradient maximum likelihood estimation, non-Gaussianity analysis, principal component analysis (PCA), linear regression model, sequential floating forward selection, or support vector machine. In some cases, an algorithm for determining an epidermal property may comprise PCA and a regression model. In some cases, an algorithm for determining an epidermal property may comprise PCA and a linear regression model. In some cases, an algorithm for determining an epidermal property may comprise PCA and a non-linear regression model.

In some cases, an algorithm for determining an epidermal property may comprise at least three of artificial neural network, Bayes classifier, blind source separation, decision tree, eigenmatrices, Gaussian radical basis function, joint-approximate diagonalization, kernel and polynomial kernel analysis, linear and nonlinear independent component analysis (ICA), natural gradient maximum likelihood estimation, non-Gaussianity analysis, principal component analysis (PCA), sequential floating forward selection, or support vector machine.

### Therapeutic Regimens

In some instances, a therapeutic regimen may comprise administration of a therapeutically effective amount of an agent that can improve or alleviate an epidermal property disclosed thereof. In some cases, a therapeutic regime may comprise various measures or procedures that are employed to a subject to treat, halt, prevent, alleviate, or improve the occurrence or manifestation of a condition of the subject.

In some cases, a therapeutic regimen may comprise administration of a therapeutically effective amount of an agent that can treat prevent a skin condition disclosed thereof. In some cases, a therapeutic regimen for administration may comprise an inorganic compound, an organic compound, a drug, a peptide, a protein, a nucleic acid, or any combination thereof.

In some cases, a therapeutic regimen may comprise application of a therapeutically effective amount of an agent that can treat prevent a skin condition disclosed thereof. In some cases, a therapeutic regimen for application may comprise an inorganic compound, an organic compound, a drug, a peptide, a protein, a nucleic acid, or any combination thereof. In some cases, an application therapeutic regimen may comprise a topical application. In some cases, a topical application may comprise a hydration mask on the skin. In some cases, an application may comprise an injection. In some cases, an injection may comprise a Botox injection. In some cases, an injection may comprise injecting deoxycholic acid, calcium hydroxylapatite, hyaluronic acid, poly-1-lactic acid, polymethylmethacrylate, bovine collagen filler, or a combination thereof. In some cases, an application may comprise chemical peel. In some cases, chemical peel may comprise applying a chemical solution to the skin that causes the skin to exfoliate and eventually peel off.

In some instances, a therapeutic regimen may comprise a physical exercise. In some cases, a physical exercise may comprise a massage of the skin. In some instances, a therapeutic regimen may comprise avoidance of exposure of an element. In some cases, an element to avoid may comprise exposure to UV light, microorganisms, food substance, wind, water, high or low temperature, or any combination thereof.

In some instances, a therapeutic regimen may comprise surgery. In some cases, a surgery may be invasive, non-invasive, or semi-invasive. In some cases, a therapeutic regimen may comprise a diet. In some cases, a surgery may comprise cryosurgery. In one case, a cryosurgery may comprise cooling the excess body fat to break down fat cells in the skin. In some cases, a surgery may also comprise a laser surgery. In some cases, a surgery may comprise dermabrasion. In some cases, dermabrasion may comprise using a wire brush or a diamond wheel with rough edges to remove and level a layer of skin. In some cases, a surgery may comprise liposuction, microlipoinjection, microneedling, micropigmentation, microwave thermolysis, neck lift, laser rejuvenation, body contouring, skin tightening, vein treatment or a combination thereof.

### Computer Systems

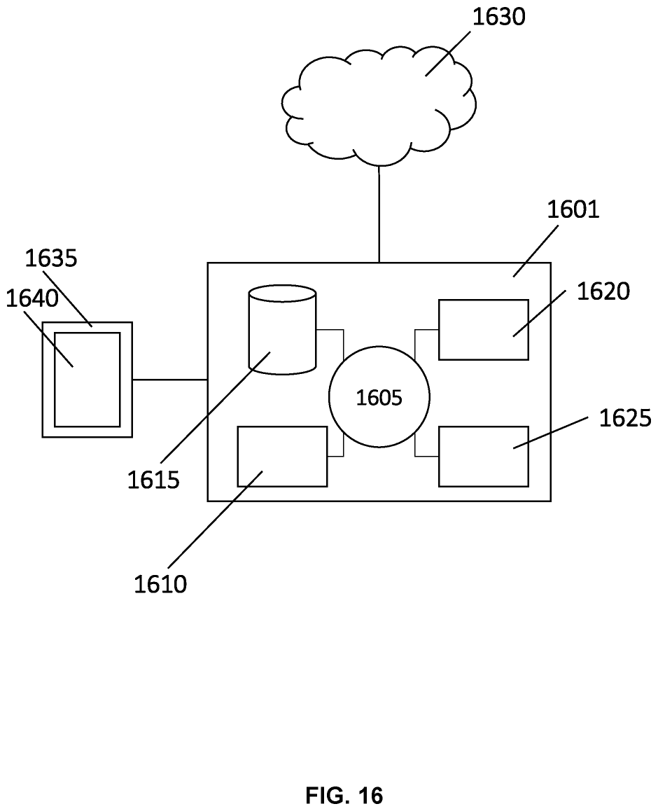

The present disclosure provides computer systems that are programmed to implement methods of the disclosure. FIG. 16 shows a computer system 1601 that is programmed or otherwise configured determine epidermal properties. The computer system 1601 can regulate various aspects of the determination of the epidermal properties of the present disclosure, such as, for example, sequencing the nucleic acid; determining the genome, epigenome, methylome, metagenome, or transcriptome; analysis of the nucleic acid; determination of the epidermal properties based at least on the nucleic acid thereof; or determination of therapeutic regimens. The computer system 1601 can be an electronic device of a user or a computer system that is remotely located with respect to the electronic device. The electronic device can be a mobile electronic device.

The computer system 1601 includes a central processing unit (CPU, also “processor” and “computer processor” herein) 1605, which can be a single core or multi core processor, or a plurality of processors for parallel processing. The computer system 1601 also includes memory or memory location 1601 (e.g., random-access memory, read-only memory, flash memory), electronic storage unit 1615 (e.g., hard disk), communication interface 1620 (e.g., network adapter) for communicating with one or more other systems, and peripheral devices 1625, such as cache, other memory, data storage and/or electronic display adapters. The memory 1601, storage unit 1615, interface 1620 and peripheral devices 1625 are in communication with the CPU 1605 through a communication bus (solid lines), such as a motherboard. The storage unit 1615 can be a data storage unit (or data repository) for storing data. The computer system 1601 can be operatively coupled to a computer network (“network”) 1630 with the aid of the communication interface 1620. The network 1630 can be the Internet, an internet and/or extranet, or an intranet and/or extranet that is in communication with the Internet. The network 1630 in some cases is a telecommunication and/or data network. The network 1630 can include one or more computer servers, which can enable distributed computing, such as cloud computing. The network 1630, in some cases with the aid of the computer system 1601, can implement a peer-to-peer network, which may enable devices coupled to the computer system 1601 to behave as a client or a server.

The CPU 1605 can execute a sequence of machine-readable instructions, which can be embodied in a program or software. The instructions may be stored in a memory location, such as the memory 1601. The instructions can be directed to the CPU 1605, which can subsequently program or otherwise configure the CPU 1605 to implement methods of the present disclosure. Examples of operations performed by the CPU 1605 can include fetch, decode, execute, and writeback.

The CPU 1605 can be part of a circuit, such as an integrated circuit. One or more other components of the system 1601 can be included in the circuit. In some cases, the circuit is an application specific integrated circuit (ASIC).

The storage unit 1615 can store files, such as drivers, libraries and saved programs. The storage unit 1615 can store user data, e.g., user preferences and user programs. The computer system 1601 in some cases can include one or more additional data storage units that are external to the computer system 1601, such as located on a remote server that is in communication with the computer system 1601 through an intranet or the Internet.

The computer system 1601 can communicate with one or more remote computer systems through the network 1630. For instance, the computer system 1601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1601 via the network 1630.

Methods as described herein can be implemented by way of machine (e.g., computer processor) executable code stored on an electronic storage location of the computer system 1601, such as, for example, on the memory 1601 or electronic storage unit 1615. The machine executable or machine readable code can be provided in the form of software. During use, the code can be executed by the processor 1605. In some cases, the code can be retrieved from the storage unit 1615 and stored on the memory 1601 for ready access by the processor 1605. In some situations, the electronic storage unit 1615 can be precluded, and machine-executable instructions are stored on memory 1601.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1601 can include or be in communication with an electronic display 1635 that comprises a user interface (UI) 1640 for providing, for example, sequencing reads of nucleic acids; sequencing analysis of nucleic acids; genomic, transcriptomic, epigenomic, methylomic, or metagenomic analysis; epidermal property determination; or therapeutic regimen determination. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1605. The algorithm can, for example, analyze or determine sequence reads; analyze or determine genome, transcriptome, epigenome, methylome, or metagenome; analyze or determine epidermal properties; or analyze or determine therapeutic regimens.

### EXAMPLES

These examples are provided for illustrative purposes only and not to limit the scope of the claims provided herein.

### Example 1: Determining Epidermal Properties from Epidermal Samples

Provided herein are schemes for determining epidermal properties of a subject.

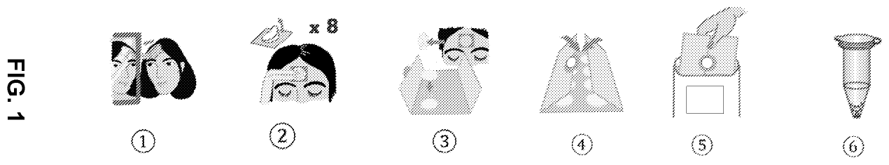

As shown in FIG. 1, to identify the epidermal properties of a human subject, the skin area (e.g., forehead) in which an epidermal sample is collected is cleaned (step #1). About 4-8 skin patches are applied to the forehead of the subject eight times (step #2). The skin patches are placed inside a liner (step #3). The liner is closed to enclose the skin patches (step #4). The liner enclosing the skin patches are saved inside a storing envelope (step #5) and sent for nucleic acid extraction (such as by the methods described in EXAMPLE 3 (step #6)). The DNA is then sequenced (such as by methods described in EXAMPLE 4). In step #6, the genome, epigenome (such as methylome), transcriptome, metagenome, or a combination thereof of the subject (such as using the methods described in EXAMPLE 4). A trained algorithm (such as any one of those described in methods of EXAMPLE 5) is used to identify the epidermal property of the subject.

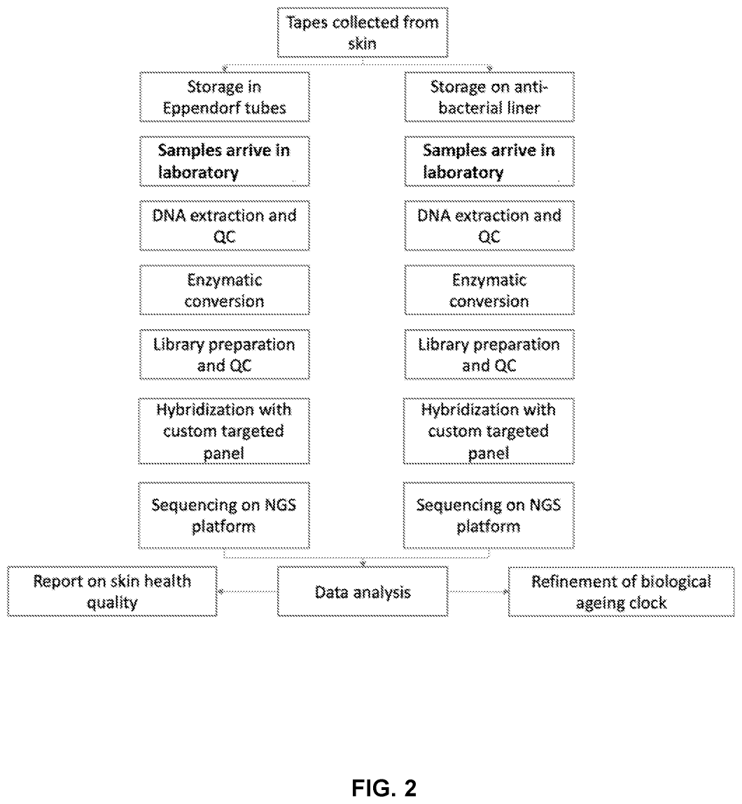

FIG. 2 shows an exemplary workflow of the processing and data analysis of the epidermal samples collected by the adhesive patch (tapes). The epidermal sample is divided and separately stored in Eppendorf tubes and anti-bacterial liner, respectively. The stored samples are sent to the laboratory for DNA extraction (such as, by the methods described in EXAMPLE 3). Once the extracted DNA passes the quality control (QC) test (such as, by the methods described in EXAMPLE 4), they are then processed via enzymatic conversion, sequencing library preparation, and the QC test. Such methods can comprise those outlined in EXAMPLE 4. A selected panel or list of sequences, such as, any one of the sites described in Table 11, is hybridized and enriched. The genome, epigenome (such as methylome), transcriptome, metagenome, or a combination thereof from the subject are analyzed (such as, using the methods described in EXAMPLE 4) via next generation sequencing (NGS). The identified sequence reads are analyzed to determine the epidermal quality of the subject, including but not limited to the biological age of the skin. The data analysis of the sequence reads can include those described in EXAMPLE 4. Determination of the epidermal properties can comprise methods described in EXAMPLEs 5-6.

### Example 2: Determining Therapeutic Regimens for Modifying Epidermal Properties

Provided herein are schemes for designing therapeutic regimens for modifying the epidermal properties of a subject.

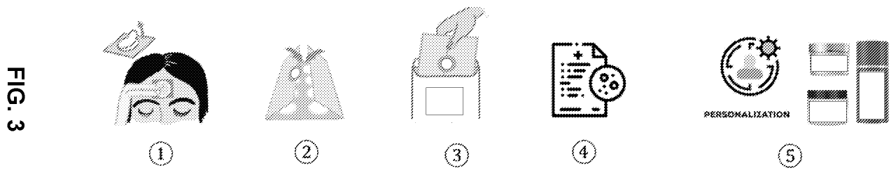

As shown in FIG. 3, skin patches are applied to the forehead of the subject eight times (step #1). The skin patches are then packaged (step #2) and sent for sequencing (step #3) using methods described in EXAMPLEs 3-4. In step #3, the genome, epigenome (such as methylome), transcriptome, metagenome, or a combination thereof of the subject using the methods described in EXAMPLE 4 (step #3). A trained algorithm (described in methods of EXAMPLE 5) is used to identify the epidermal property of the subject (step #4). Based on the epidermal property of the subject, a personalized therapeutic regimen specific for the subject is designed to improve or alleviate the skin property using methods described in EXAMPLE 6 (step #5).

### Example 3: DNA Extraction from Epidermal Samples

Provided herein is a protocol for extracting DNA from epidermal samples collected with skin patches.

### Tissue Lysis and Degradation

95 microliter (μL) elution buffer (10 mM Tris, pH 8.5, 0.1 mM EDTA), 95 μL Biofluid & Solid Tissue buffer (Biofluid & Solid Tissue buffer, Catalog #: D4081-3), and 10 μL Protein Kinase (20 mg Protein Kinase powder in 1140 μL Protein Kinase Storage Buffer for a concentration of 20 ng/μL) were added to the skin patch containing the epidermal sample in a 2 mL tube. The tube was vortexed briefly for maximal contact between the buffer solution and the patch/sample. The mixture was incubated at 500 rotation per minute (rpm) at 55° C. for one hour and 30 minutes. The mixture content of each of the eight tubes was mixed in one Eppendorf tube, which constituted a sample. The sample was then incubated at 1300 rpm at 55° C. for 30 minutes.

### DNA Extraction

The mixture for each sample was transferred to an Eppendorf tube. Quick DNA MagBinding Buffer (Zymo Research, catalog #: D6060) was added to the sample and mixed by pipetting. A volume ratio of 1:1 of sample: Quick DNA MagBinding Buffer was required. 50 μL MagBinding Beads (Zymo Research, catalog #: D6060) were then added to the mixture and mixed by pipetting five times. The sample tube was placed on a shaker and incubated at 1300 rotation per minute for 10 minutes at room temperature and then transferred to the magnetic stand until the beads had separated from the solution and formed a pellet. The supernatant was removed, and the sample tube was transferred off the magnetic stand.

700 μL Quick-DNA MagBinding Buffer (Zymo Research, catalog #: D6060) was used to resuspend the beads by pipetting five times. The sample tube was placed on the shaker and incubated at 1300 rpm for 5 minutes at room temperature. The supernatant was removed, and the sample tube was transferred off the magnetic stand.

700 μL DNA Pre-Wash Buffer (Zymo Research, catalog #: D6060) was used to resuspend the beads by pipetting 10 times. The sample tube was transferred to the magnetic stand until the beads had separated from the solution and formed a pellet. The supernatant was removed, and the sample tube was transferred off the magnetic stand.

The following steps were repeated two times: 900 μL DNA Wash Buffer was used to resuspend the beads by pipetting 10 times. The sample tube was transferred to a new microcentrifuge tube or 96-well plate. The new tube/well was transferred to the magnetic stand until the beads had separated from the solution and formed a pellet. The supernatant was removed, and the sample tube/well was transferred off the magnetic stand.

Between each wash, the sample was allocated into a new Eppendorf tube.

To dry the beads, the sample was transferred to a magnetic element and incubated for 10 mins. Alternatively, the beads could be air-dried for 20 mins.

The following steps were repeated two times: DNA Extraction Buffer was then added to the dried mixture. The mixture was placed on the shaker for five mins at room temperature. The sample tube was transferred to the magnetic stand until the beads had separated from the solution and formed a pellet.

The eluted DNA can be used immediately or stored at −20° C. Each sample can include eight subsequent skin patches collected from the same area on the forehead. On average, at least about 40 ng purified high molecular weight (HMW) DNA could be obtained per sample.

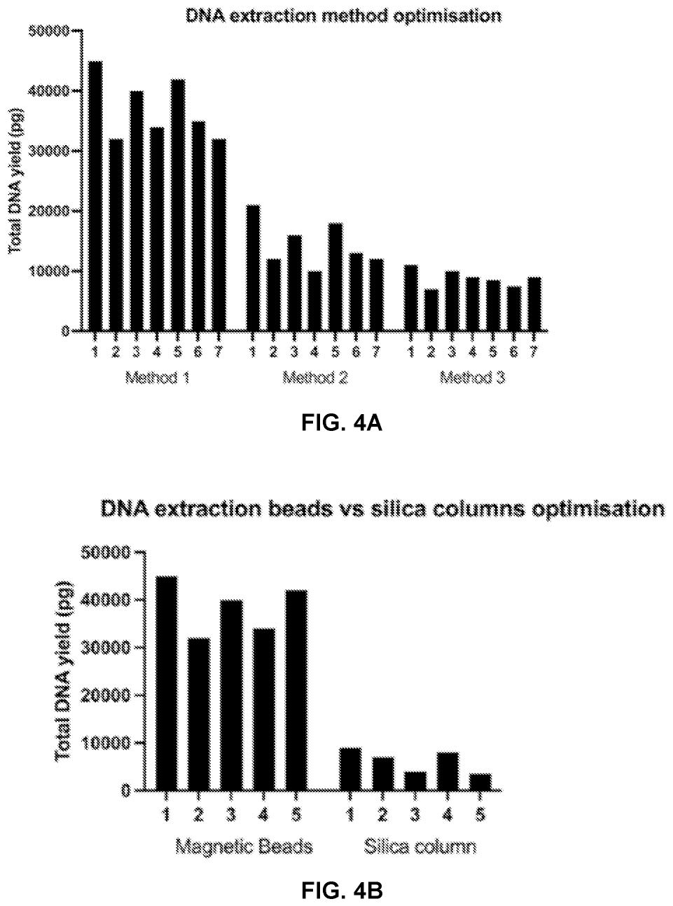

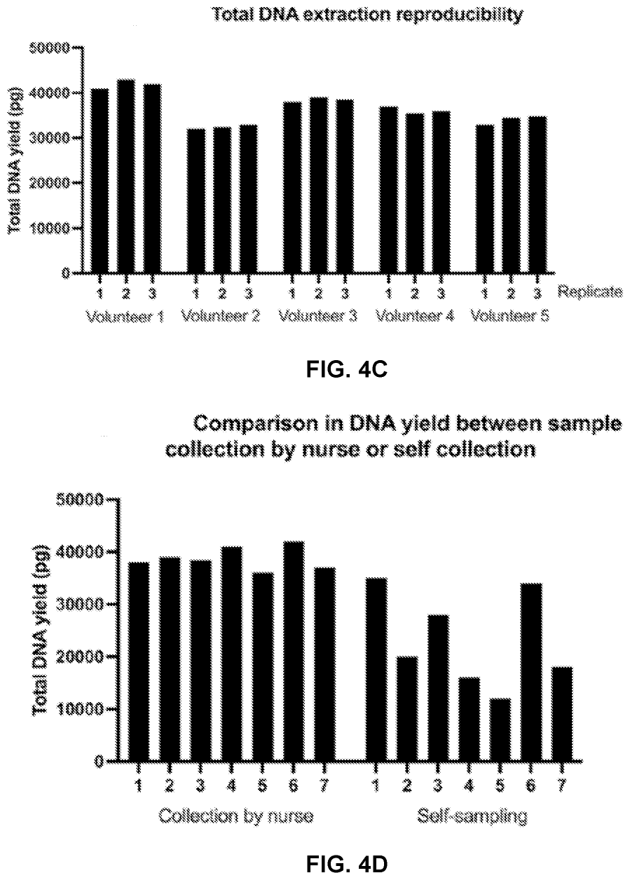

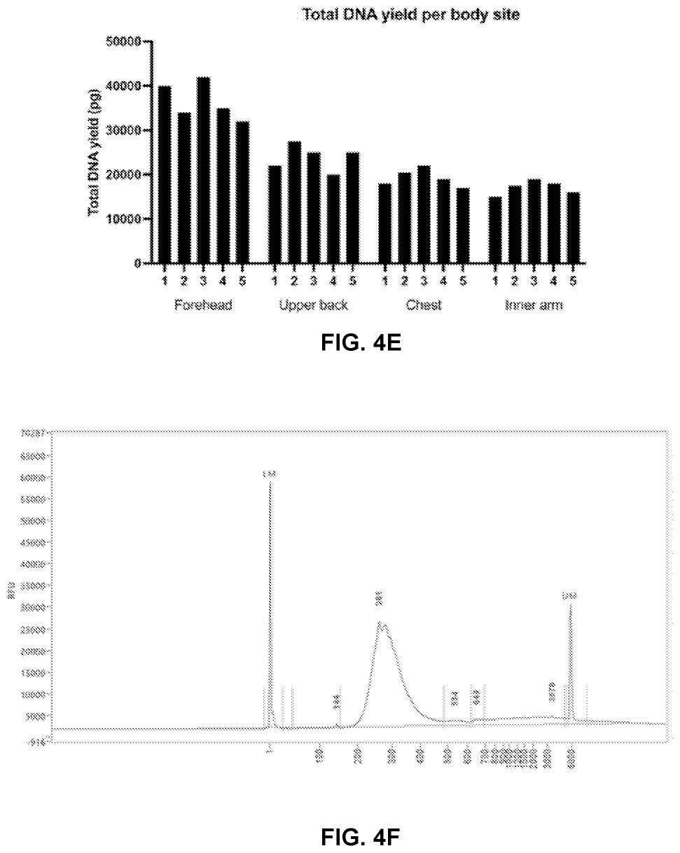

FIGS. 4A-F show the advantages of the DNA extraction method described herein in this EXAMPLE. As shown in FIG. 4A, the DNA extraction method described in this EXAMPLE (Method 1) consistently extracted more DNA, relative to those obtained by other methods (Method 2 and 3, which used extraction kit from Qiagen and ThermoFisher, respectively). Every replicate (labeled as 1-7 on the x-axis of FIG. 4A) of Method 1 yielded more DNA than any replicates of Methods 2 and 3. FIG. 4B shows that the DNA extraction method described in this EXAMPLE using magnetic beads to bind and elute DNA extracted at least 3 times more DNA than a similar method that uses silica column. FIG. 4C shows that the DNA extraction method described in this EXAMPLE is reproducible. The method extracted similar amounts of DNA from different replicates (labeled as 1-3 on the x-axis) from the same subject (volunteer). Further, the method also extracted similar amounts of DNA from different subjects. FIG. 4D shows that the DNA extraction method described in this EXAMPLE extracted sufficient amounts of DNA, whether the epidermal sample had been collected by a nurse or by a subject (self-sampling). FIG. 4E shows that the DNA extraction method described in this EXAMPLE extracted sufficient amounts of DNA from different body parts. FIG. 4F shows a quality control (QC) analysis of the DNA extracted by the method described in this EXAMPLE. Fragmentation of DNA was performed on a Covaris or Pixul instrument. The QC analysis was performed on a Fragment Analyser instrument. A fragment size of 240-290 bp was preferred for the sequencing of the epidermal sample, such as using the method described in EXAMPLE 4 or elsewhere in the disclosure. These results show that the DNA extraction method described in this EXAMPLE is reproducible. It can also extract sufficient amount of DNA for downstream analysis, such as sequencing, whether the epidermal sample is extracted by a professional (e.g., a nurse) or via self-sampling.

### Example 4: Sequencing Epidermal Sample

Provided herein are methods for sequencing the epidermal sample of a subject obtained by a skin patch and methods for analyzing sequence reads.

### Enzymatic Methyl Sequencing (EM-seq)

The methylated nucleotide/sequence of the DNA was identified by enzymatic methyl sequencing using the reagents of NEBNext® Enzymatic Methyl-seq kit (NEB #E7120S/L).

About 10-100 ng of sheared DNA was subjected to two sets of enzymatic conversion steps to differentiate unmethylated cytosines from 5mC and 5hmC. The libraries are then PCR amplified before subjected to sequencing.

The two enzymatic processes result in the deamination of the unmethylated cytosines to uracils. The first conversion step uses TET2 to convert 5mC to 5hmC, and subsequently to 5fC, and then 5caC; and E. coli's UDP-glucose:DNA beta-D-glucosyltransferase to convert 5hmc to 5ghmC. The second conversion step uses APOBEC3A to deaminate unmodified cytosines. However, 5caC and 5ghmC are resistant to deamination. During a PCR reaction of the converted DNA, 5caC and 5ghmC are converted to C while U is converted to T, Hence, in a sequencing reaction, unmethylated C is identified as T; methylated C is identified as C. By comparing these sequences to reference sequences (C/T and G/A converted genome), the methylation sequence/residue is identified.

To prepare DNA for enzymatic methyl sequencing, the following protocol was carried out.

### 1. DNA Preparation

Genomic DNA (10-100 ng) with control DNAs, CpG methylated pUC19 (lilac) and unmethylated lambda DNA (lilac) were combined. in 50 μL made up with 0.1×TE pH 8.0. The amount of control DNA added was dependent on the number of reads required. If checking library quality on a MiSeq® (2-4 M reads per library) prior to deep sequencing on NovaSeq®, HiSeq® or NextSeq® (100-500 M reads per library) then the amount of controls spiked to the sample DNA is higher than what is required for direct deep sequencing. Having higher amount of control DNA for samples that are sequenced on a MiSeq ensures that there are enough control reads to accurately call cytosine conversion. For libraries sequenced to a depth of 2-4 million paired end reads, approximately 5,000×76 base paired end reads of unmethylated lambda and 500×76 base paired end reads of CpG methylated pUC19 are needed to give enough reads for accurate conversion estimates. If these same libraries are sequenced to a higher depth of 200-400 M reads per library, then the number of reads associated with the controls would be in vast excess, 500,000 for unmethylated lambda and 50,000 for pUC19.

### 2. Shearing DNA

The combined 50 μL genomic DNA and control DNAs were fragmented to an average insert size of 240-290 bp (370-420 bp final Illumina library). The combined 50 μL genomic DNA and control DNAs can also be fragmented to an average insert size of 350-400 bp (470-520 bp final Illumina library) depending on the application. Fragmentation could be done using a preferred fragmentation device such as a Covaris instrument. Enzymatic fragmentation is not recommended as this step may result in the removal of methylation marks. The 50 μL of sheared DNA was transferred to a new PCR tube for End Prep.

The following components were mixed in a sterile nuclease-free PCR tube according to TABLE 1.

TABLE 1
Recipe for a PCR Reaction for Fragmented DNA

| Component | Volume |
|---|---|
| Fragmented DNA | 50 μL |
| (green) NEBNext Ultra II End Prep Reaction Buffer | 7 μL |
| (green) NEBNext Ultra II End Prep Enzyme Mix | 3 μL |
| Total Volume | 60 μL |

A 100 μL or 200 μL pipette was set to 50 μL and then the entire volume was pipetted up and down at least 10 times to mix thoroughly. A quick spin was performed to collect all liquid from the sides of the tube. It is important to mix well. The presence of a small amount of bubbles does not interfere with the performance. The PCR tube was then placed in a thermocycler with the heated lid set to at least 75° C. A PCR amplification program (30 minutes at 20° C., 30 minutes at 65° C., and hold at 4° C.) was run.

### 3. Ligation of EM-Seq Adaptor

On ice, the components listed in TABLE 2 was added directly to the 60 μL End Prep reaction mixture and mixed thoroughly.

TABLE 2
Recipe for Ligation Reaction of EM-seq Adaptor

| Component | Volume | |
|---|---|---|
| (red) NEBNext EM-seq Adaptor | 2.5 | μL |
| (red) NEBNext Ligation Enhancer | 1 | μL |
| (red) NEBNext Ultra II Ligation Master Mix | 30 | μL |
| Total Volume | 93.5 | μL |

Ligation Enhancer and Ligation Master Mix can be mixed ahead of time and is stable for at least 8 hours at 4° C. Adaptor and sample were premixed and the other ligation reagents were added.

A 100 μL or 200 μL pipette was set to 80 μL and then the entire volume was pipetted up and down 10 times to mix thoroughly. A quick spin was performed to collect all liquid from the sides of the tube. Adequate mixing of the ligation reaction, as incomplete mixing will result in reduced ligation efficiency. The presence of a small amount of bubbles does not interfere with performance.

The mixture was incubated at 20° C. for 15 minutes in a thermocycler with the heated lid off.

### 4. Clean-Up of Adaptor Ligated DNA

Sample Purification Beads were vortexed to resuspend. 90 μL of resuspended NEBNext Sample Purification Beads was added to each sample and mixed well by pipetting up and down at least 10 times. Samples were incubated on bench top for at least 5 minutes at room temperature. The tubes were placed against an appropriate magnetic stand to separate the beads from the supernatant. After 5 minutes or when the solution was clear, the supernatant was carefully removed and discarded without disturbing the beads that contained DNA targets. 200 μL of 80% freshly prepared ethanol was added to the tubes while in the magnetic stand and incubated at room temperature for 30 seconds. The supernatant was carefully removed and discarded the supernatant without disturbing the beads that contained the DNA targets. The was repeated once for a total of two washes. All visible liquid was removed after the second wash using a p10 pipette tip. The beads were air-dried for up to 2 minutes while the tubes were on the magnetic stand with the lid open. The samples were eluted when the beads were still dark brown and glossy looking, but when all visible liquid had been evaporated. The tubes were removed from the magnetic stand. The DNA targets were eluted from the beads by adding 17 μL of Elution Buffer (white). The samples were mixed well by pipetting up and down 10 times and incubated for at least 1 minute at room temperature. The sample could be quickly spun to collect the liquid from the sides of the tube before placing back on the magnetic stand. The tube was placed on the magnetic stand. After 3 minutes or whenever the solution became clear, 16 μL of the supernatant was transferred to a new PCR tube.

### 5. Oxidation of 5-Methylcytosines and 5-Hydroxymethylcytosines

TET2 Buffer was prepared. The TET2 Reaction Buffer Supplement is a powder and was centrifuged before use to ensure it was at the bottom of the tube. For E7120S/E7120G (24 reactions/G size), 100 μL of TET2 Reaction Buffer was added to one tube of TET2 Reaction Buffer Supplement and mixed well. For E7120L (96 reactions), 400 μL of TET2 Reaction Buffer to was added one tube of TET2 Reaction Buffer Supplement and mix well.

The components listed in TABLE 3 was added directly to the 28 μL EM-seq adaptor ligated DNA.

TABLE 3
Recipe for Oxidation Reaction Mix of 5-Methylcytosines
and 5-Hydroxymethylcytosines of DNA

| Component | Volume |
|---|---|
| (yellow) TET2 Reaction Buffer (TET2 Reaction Buffer | 10 μL |
| plus reconstituted TET2 Reaction Buffer Supplement) | |
| (yellow) Oxidation Supplement | 1 μL |
| (yellow) DTT | 1 μL |
| (yellow) Oxidation Enhancer | 1 μL |
| (yellow) TET2 | 4 μL |

The sample was mixed thoroughly by vortexing and centrifuged briefly. For multiple reactions, a master mix of the above reaction components could be prepared before addition to the sample DNA. 5mC/5hmC oxidation was initiated by the addition of the Fe(II) solution. 500 mM Fe(II) Solution (yellow) was diluted by adding 1 μL to 1,249 μL of water. The diluted Fe(II) Solution and EM-seq DNA were combined with Oxidation Enzymes according to TABLE 4.

TABLE 4
Recipe for Oxidation Mix, DNA, and Fe(II) Solution

| Component | Volume |
|---|---|
| EM-seq DNA (from Step 1.5.2) | 45 μL |
| Diluted Fe(II) Solution (from Step 1.5.3) | 5 μL |
| Total Volume | 50 μL |

The sample was mixed thoroughly by vortexing or by pipetting up and down at least 10 times and centrifuged briefly. The mixture was incubated at 37° C. for 1 hour in a thermocycler with the heated lid set to >45° C. or on. The sample was then transferred to ice and 1 μL of Stop Reagent (yellow) was added.

The sample was mixed thoroughly by vortexing or by pipetting up and down at least 10 times and centrifuge briefly. The mixture was incubated at 37° C. for 30 minutes then at 4° C. in a thermocycler with the heated lid set to >45° C. or on.

### 6. Clean-Up of TET2 Converted DNA

Sample Purification Beads was vortexed to resuspend. 90 μL of resuspended NEBNext Sample Purification Beads was added to each sample. The sample was mixed well by pipetting up and down at least 10 times. The samples were then incubated on bench top for at least 5 minutes at room temperature. The tubes were placed against an appropriate magnetic stand to separate the beads from the supernatant. After 5 minutes or when the solution is clear, the supernatant was carefully removed and discarded. 200 μL of 80% freshly prepared ethanol was added to each tube while in the magnetic stand. The sample was incubated at room temperature for 30 seconds. The supernatant was carefully removed and discarded. The wash was repeated once for a total of two washes. The beads were air-dried for up to 2 minutes while the tubes were on the magnetic stand with the lid open. The tubes were removed from the magnetic stand. The DNA target was eluted from the beads by adding 17 μL of Elution Buffer (white). The sample was mixed well by pipetting up and down 10 times. The mixture was incubated for at least 1 minute at room temperature. If necessary, the sample could be quickly spun to collect the liquid from the sides of the tube before placing back on the magnetic stand. The tube was placed on the magnetic stand. After 3 minutes or whenever the solution is clear, 16 μL of the supernatant was transferred to a new PCR tube.

### 7. Deamination of Cytosines

The DNA were denatured using either Formamide or 0.1 N sodium hydroxide. For using formamide, a thermocycler was pre-heated to 85° C. with the lid on. 4 μL Formamide was added to the 16 μL of oxidized DNA. The mixture was vortexed to mix or pipetted up and down at least 10 times and then centrifuged briefly. The mixture was incubated at 85° C. for 10 minutes in the pre-heated thermocycler with the heated lid on and immediately placed on ice afterwards. For sodium hydroxide, 0.1 N NaOH was freshly diluted. A thermocycler was pre-heated to 50° C. with the lid on. 4 μL 0.1 N NaOH was added to the 16 μL of oxidized DNA. The mixture was vortexed to mix or pipetted up and down at least 10 times and then centrifuged briefly. The mixture was incubated at 50° C. for 10 minutes in the pre-heated thermocycler with the heated lid on and immediately placed on ice afterwards.

The components listed in TABLE 5 was added to the 20 μL of denatured DNA.

TABLE 5
Recipe for a Cytosine Deamination Reaction of DNA

| Component | Volume | |
|---|---|---|
| Nuclease-free water | 68 | μL |
| (orange) APOBEC Reaction Buffer | 10 | μL |
| (orange) BSA | 1 | μL |
| (orange) APOBEC | 1 | μL |
| Total Volume | 100 | μL |

The mixture vortexed or by pipetted up and down at least 10 times and centrifuged briefly. The thoroughly mixed mixture was incubated at 37° C. for 3 hours then at 4° C. in a thermocycler with the heated lid set to ≥45° C. or on.

### 8. Clean-Up of Deaminated DNA

Sample Purification Beads were vortexed to resuspend. 100 μL of resuspended NEBNext Sample Purification Beads was added to each sample. The samples were mixed well by pipetting up and down at least 10 times. The samples were then placed on bench top for at least 5 minutes at room temperature. The tubes were placed against an appropriate magnetic stand to separate the beads from the supernatant. After 5 minutes or when the solution is clear, the supernatant was carefully removed and discarded. 200 μL of 80% freshly prepared ethanol was added to the tubes while in the magnetic stand. The mixture was incubated at room temperature for 30 seconds, with the supernatant carefully removed and discarded. The wash was repeated once for a total of two washes. All visible liquid was removed after the second wash using a p10 pipette tip. The beads were air-dried for up to 90 seconds while the tubes were on the magnetic stand with the lid open. The tubes were removed from the magnetic stand. The DNA target was eluted from the beads by adding 21 μL of Elution Buffer (white). The mixture was pipetted up and down 10 times and incubated for at least 1 minute at room temperature. The tube was placed on the magnetic stand. After 3 minutes (or whenever the solution is clear), 20 μL of the supernatant was transferred to a new PCR tube.

### 9. PCR Amplification of Deaminated DNA

The following components were mixed in a sterile nuclease-free PCR tube according to TABLE 6.

TABLE 6
Recipe for a PCR Reaction for Deaminated DNA

| Component | Volume |
|---|---|
| EM-seq Index Primer* | 5 μL |
| (blue) NEBNext Q5U Master Mix | 25 μL |
| Total Volume | 50 μL |

*EM-seq primers are supplied in tubes in NEB #E7120S or as a 96 Unique Dual Index Primers Pairs Plate in NEB #E7120L

The PCR mix was mixed thoroughly by vortexing or by pipetting up and down at least 10 times. The mixture was centrifuged briefly. The tube was placed in a thermocycler and perform PCR amplification using the following cycling conditions according to TABLE 7.

TABLE 7
PCR Cycles for Deaminated DNA PCR

| CYCLE STEP | TEMP | TIME | | CYCLES |
|---|---|---|---|---|
| Initial Denaturation | 98° C. | 30 | seconds | 1 |
| Denaturation | 98° C. | 10 | seconds | 4-8* |
| Annealing | 62° C. | 30 | seconds | |
| Extension | 65° C. | 60 | seconds | |
| Final Extension | 65° C. | 5 | minutes | 1 |

| | | | |
|---|---|---|---|
| Hold | 4° C. | ∞ | N/A |

Cycle Recommendations: 10 ng DNA input: 8 cycles; 50 ng DNA input: 5-6 cycles; 200 ng DNA input: 4 cycles

The number of cycles of amplification could be optimized to account for initial sample concentration.

### 10. Clean-Up of Amplified Libraries

Sample Purification Beads were vortexed to resuspend. 45 μL of resuspended NEBNext Sample Purification Beads was added to each sample. The mixture was mixed thoroughly by pipetting up and down at least 10 times. The samples were incubated on bench top for at least 5 minutes at room temperature. The tubes were placed against an appropriate magnetic stand to separate the beads from the supernatant. After 5 minutes or when the solution was clear, the supernatant was removed and discarded. 200 μL of 80% freshly prepared ethanol was added to the tubes while in the magnetic stand. The mixture was incubated at room temperature for 30 seconds. The supernatant was then carefully removed and discarded. The wash was repeated once for a total of two washes. The beads were air-dried for up to 2 minutes while the tubes were on the magnetic stand with the lid open. The samples were eluted when the beads were still dark brown and glossy looking, but when all visible liquid had been evaporated. The tubes were removed from the magnetic stand. The DNA target was eluted from the beads by adding 21 μL of Elution Buffer (white) or 21 μL of TE (10 mM Tris, 0.1 mM EDTA, pH 8.0) or low TE (for long term storage). The sample was mixed by pipetting up and down 10 times. The mixture was incubated for at least 1 minute at room temperature. The tube was placed on the magnetic stand. After 3 minutes (or whenever the solution is clear), 20 μL of the supernatant was transferred to a new PCR tube. The sample was then quantified using a Bioanalyzer or TapeStation to determine the size distribution and concentration of the libraries.

### 11. Quantification of Sequencing Reads by Enzymatic Methyl Sequencing

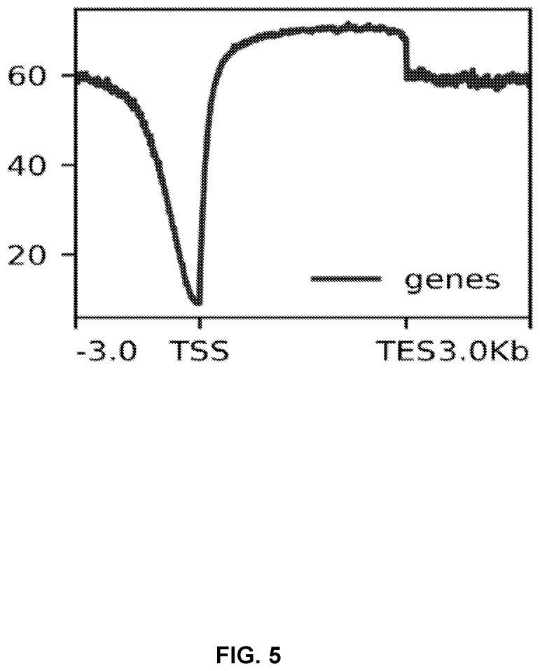

Enzymatic methyl sequencing generally does not cause damages to the DNA, in contrast to the bisulfide treatment used in bisulfide sequencing. Robust DNA methylation is also observed using enzymatic methyl sequencing using NextSeq 30X sequencing platform from epidermal sample obtained from skin patches. As shown in FIG. 5, the sequencing identifies a majority of DNA methylation occurred around the transcription start site (TSS), similar to those identified by other sequencing methods. The data suggest that enzymatic methyl sequencing is robust enough to detect methylation or epigenetic mark using epidermal sample identified from skin patches.

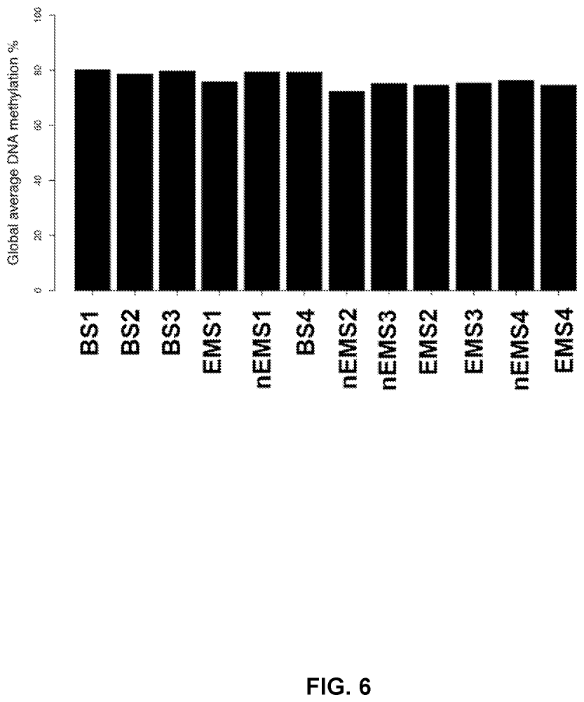

FIG. 6 shows a comparison of DNA methylation patterns between bisulfite conversion and enzymatic conversion derived from epidermal samples. The epidermal sample was collected via biopsy or non-invasively by applying eight adhesive skin patches on the forehead area of the subject. DNA extraction was performed as described in EXAMPLE 3. Epidermal samples labelled with “EMS” and “nEMS” were processed by enzymatic conversion as described by the method described in this EXAMPLE for methylation sequencing. Samples labelled with “BS” s were processed by bisulfite conversion for methylation sequencing. All samples were sequenced on an Illumina MiSeq instrument. EMS and BS were biopsy samples. nEMS was sample obtained non-invasively. Different replicates (#1-#5) were included for comparisons. Global average DNA methylation was calculated from the sequencing data. As shown in FIG. 6, there was no statistical difference between the two methods. Hence, the data suggest that the method described in this EXAMPLE generates a similar global DNA methylation profile, relative to that generated by the traditional bisulfite conversion method.

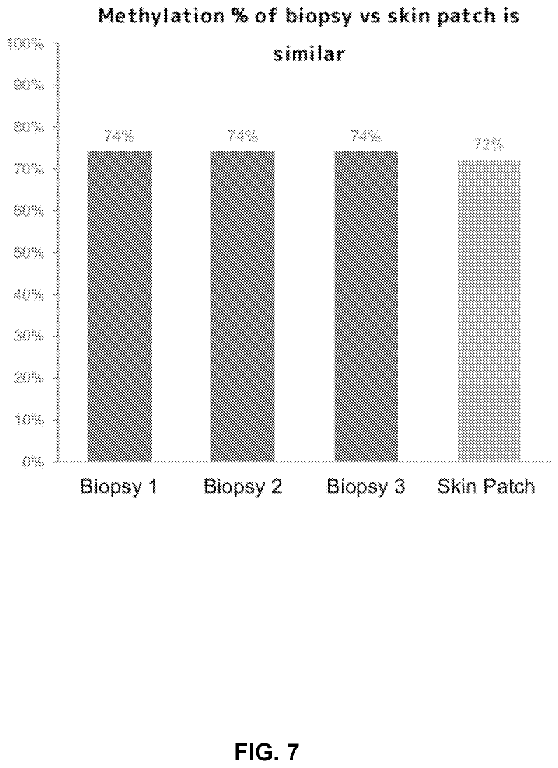

Furthermore, as shown in FIG. 7 and TABLE 8, when compared to regular punch biopsy, enzymatic methyl sequencing identifies a similar proportion of methylated DNA in the epidermal samples obtained from skin patches, suggesting that this sequencing method and the non-invasive skin patch are sufficient to identify the epidermal methylome of a subject.

TABLE 8
Summary of the Proportion of Methylated CpG Sites
in Epidermal Samples Obtained by Different Methods

| | Proportion of CpG Sites Methylated |
|---|---|
| Epidermal Sampling Methods | Identified by Sequencing |
| Biopsy 1 | 74% |
| Biopsy 2 | 74% |
| Biopsy 3 | 74% |
| Skin Patch | 72% |

### Sequence Analysis

To characterize a genome, the DNA sequence reads of an epidermal sample is compared to a reference human genome sequence. The mutation or variations of the DNA sequence relative to the reference sequence can be identified.

The methylome of the epidermal sample can be identified by enzymatic methyl sequencing.

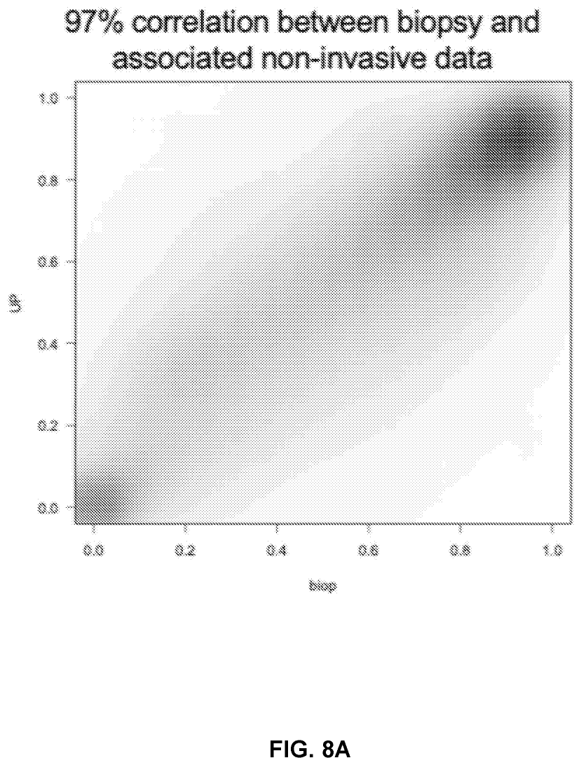

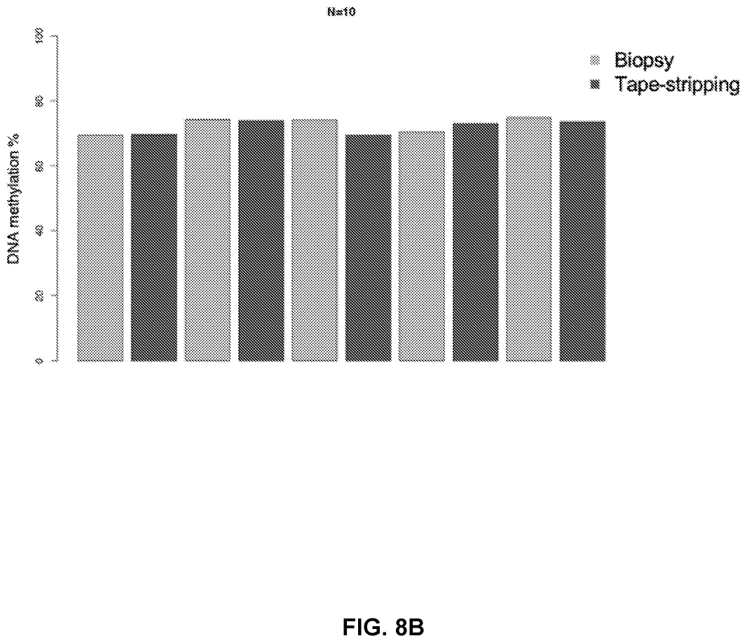

FIGS. 8A-8B show a comparison in the DNA methylation landscape between biopsy (invasive) and non-invasive sampling from the same individual. Invasive samples consisting of skin biopsies were collected from the upper back from five subjects. Non-invasive samples consisting of eight adhesive skin tapes were collected from an adjacent spot near the biopsy site. DNA extraction was performed as described in EXAMPLE 3. Approximatively 40 ng of the extracted DNA was processed as described in EXAMPLE 4. All samples were sequenced on an Illumina MiSeq instrument. All Global average DNA methylation was calculated from sequencing data. The DNA methylation landscape obtained using the non-invasive sampling method was comparable to that obtained using the invasive biopsy. There was 97% correlation in methylation data derived from the biopsy method compared to those derived from the tape-stripping method (x-axis indicates DNA methylation from upper back biopsy, y-axis indicates DNA methylation from upper back adhesive tape collection (FIG. 8A). There was no significant difference between the DNA methylation % derived from tape stripping or biopsy (FIG. 8B). Different bars represent different samples from the five subjects.

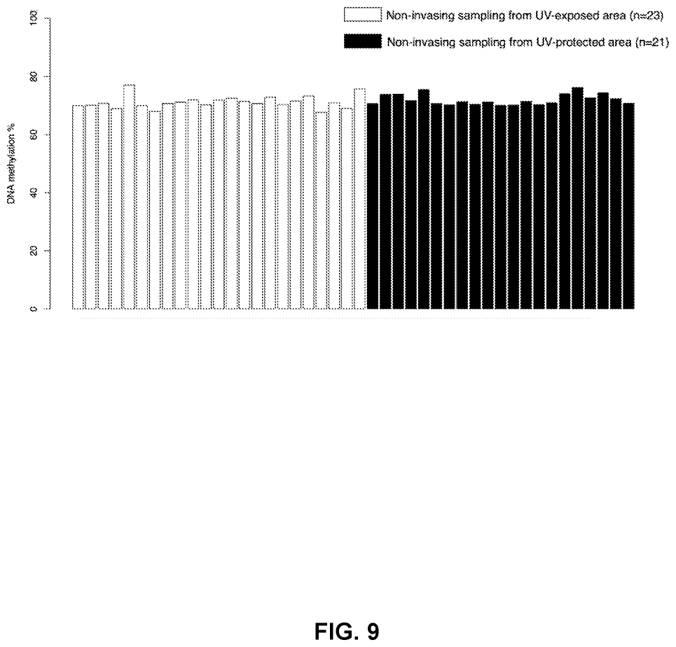

FIG. 9 shows the reproducibility of the DNA methylation patterns collected by non-invasive sampling from forehead and upper back from different subjects. Non-invasive samples consisting of eight adhesive skin tapes were collected from the same spot from two anatomical body sites: forehead and upper-back. DNA extraction was performed as described in EXAMPLE 3. Approximatively 40 ng of the extracted DNA was processed as described in EXAMPLE 4. All samples were sequenced on an Illumina MiSeq instrument. Global DNA methylation was calculated from the sequencing data. There was no statistical difference observed between sample replicates (represented by individual bars).

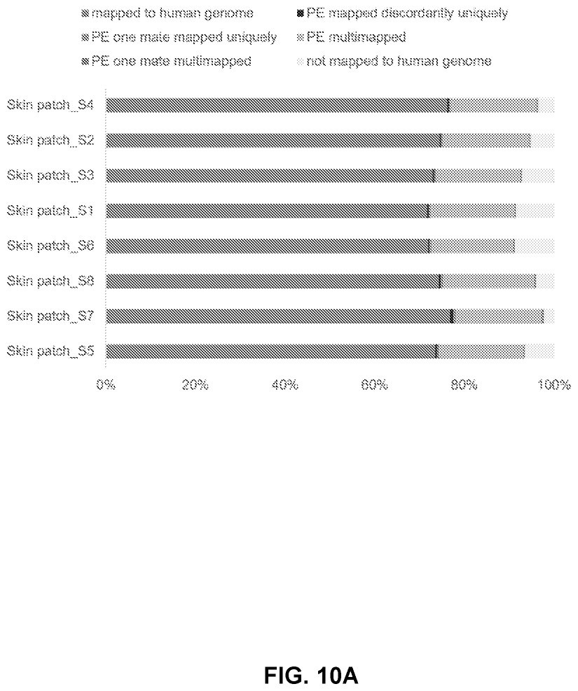

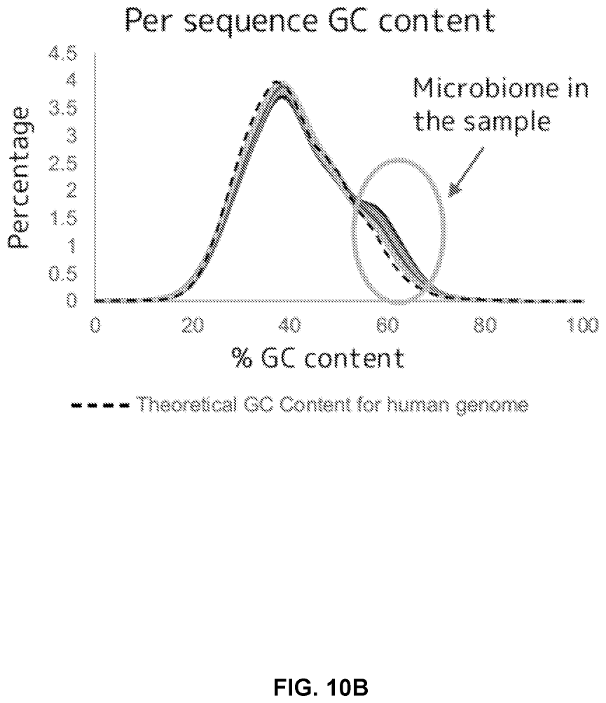

The metagenome of the epidermal sample can be identified by the identification of the sequences that are not mapping to the human genome. As shown in FIG. 10A and TABLES 9-10, mapping the sequence reads of the whole-genome sequencing of multiple epidermal samples obtained by skin patches identifies ˜90% the sequence reads, including pair-end (PE) mate sequence reads, that can be mapped individually or multiply to the human genome. About 10% of the sequence reads not mappable to the human genome represents the epidermal metagenome of the subject.

TABLE 9
Summary of the Total Numbers of Sequence Reads Mapped

| Skin | | PE mapped | PE one mate | | | PE neither | |
|---|---|---|---|---|---|---|---|
| Patch | PE mapped | discordantly | mapped | PE | PE one mate | mate | |
| Sample | uniquely | uniquely | uniquely | multimapped | multimapped | aligned | Total |
| 5 | 3208136 | 16375 | 20394.5 | 822436 | 10673 | 286711.5 | 4364726 |
| 7 | 4069911 | 42590 | 26483 | 1020007 | 17365 | 125252 | 5301608 |
| 8 | 3217594 | 20673 | 23645.5 | 887956 | 12286.5 | 175740 | 4337895 |
| 6 | 3702152 | 18892 | 22164 | 944855 | 11549.5 | 454157.5 | 5153770 |
| 1 | 2741322 | 11267 | 16695 | 717802 | 8107 | 327996 | 3823189 |
| 3 | 2922188 | 11864 | 16450.5 | 759810 | 7977 | 287517.5 | 4005807 |
| 2 | 2622681 | 10469 | 12956.5 | 682387 | 6320.5 | 188015 | 3522829 |
| 4 | 2494678 | 9546 | 13396.5 | 631877 | 6505 | 120006.5 | 3276009 |

TABLE 10
Summary of the Proportion of Sequence Reads Mapped

| Skin | | PE mapped | PE one mate | | | PE neither | |
|---|---|---|---|---|---|---|---|
| Patch | PE mapped | discordantly | mapped | PE | PE one mate | mate | |
| Sample | uniquely | uniquely | uniquely | multimapped | multimapped | aligned | Total |
| 5 | 74% | 0.38% | 0.47% | 18.84% | 0.24% | 6.57% | 74% |
| 7 | 77% | 0.80% | 0.50% | 19.24% | 0.33% | 2.36% | 77% |
| 8 | 74% | 0.48% | 0.55% | 20.47% | 0.28% | 4.05% | 74% |
| 6 | 72% | 0.37% | 0.43% | 18.33% | 0.22% | 8.81% | 72% |
| 1 | 72% | 0.29% | 0.44% | 18.77% | 0.21% | 8.58% | 72% |
| 3 | 73% | 0.30% | 0.41% | 18.97% | 0.20% | 7.18% | 73% |
| 2 | 74% | 0.30% | 0.37% | 19.37% | 0.18% | 5.34% | 74% |
| 4 | 76% | 0.29% | 0.41% | 19.29% | 0.20% | 3.66% | 76% |

Consistent with this analysis, as shown in FIG. 10B, a portion of the total sequences has an elevated GC content relative to that of a theoretical GC content distribution calculated using the reference human sequences, suggesting that these sequences are the epidermal metagenome of the subject. These potential metagenome sequences can then be characterized by comparing to the reference human microbiome sequence. Metagenomic reads are aligned to a database consisting of species associated with the skin microbiome. For example, the potential metagenome sequence may be compared to reference microbial 16S rDNA sequences.

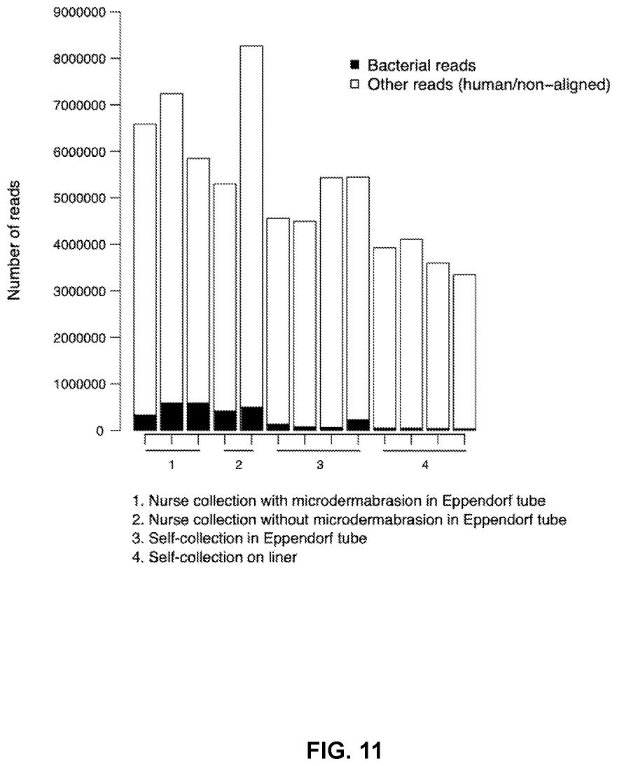

FIG. 11 shows a comparison of both human DNA and bacterial DNA collected by non-invasive sampling. Non-invasive samples consisting of eight adhesive skin tapes collected from the same spot on the forehead. DNA extraction was performed as described in EXAMPLE 3. Approximatively 40 ng of the extracted DNA was processed as described in EXAMPLE 4. All samples were sequenced on an Illumina MiSeq instrument. Total sequencing reads of human DNA and bacterial DNA were investigated. Different sampling conditions were assessed: nurse sample collection vs self-sampling; with microdermabrasion vs without microdermabrasion, and storage in Eppendorf tube vs storage on liner. There were more sequencing reads derived from samples collected by a trained professional (e.g., a nurse). However, there was no significant difference between treating the skin surface with microdermabrasion or not. In the self-collected samples, there were more bacterial reads identified in the samples stored in Eppendorf tubes compared to those stored on a liner.

### Example 5: Identifying Epidermal Properties

Provided herein are methods for identifying epidermal properties from sequence reads obtained from epidermal samples.

A trained algorithm is created using reference sequence data, genome, transcriptome, methylome, or metagenome with known epidermal properties. Epidermal sequence reads from a subject identified from methods described in EXAMPLE 1, 3, and 4 are analyzed using the trained algorithm to predict the epidermal properties associated with the sequence reads.

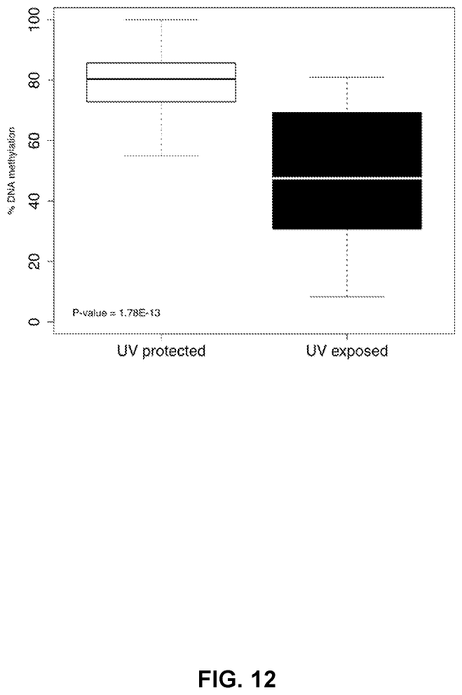

FIG. 12 shows methylation differences between UV-exposed and UV-protected epidermal samples collected non-invasively by applying eight adhesive skin tapes on the same spot from two anatomical body sites: forehead (UV exposed) and upper-back (UV protected). DNA extraction was performed as described in EXAMPLE 3. Approximatively 40 ng of the extracted DNA was processed as described in EXAMPLE 4. All samples were sequenced on an Illumina NovaSeq instrument. The sites that showed significant methylation changes between the upper back and forehead samples were identified by running a linear regression model using DNA methylation as the outcome, tissue type as independent variable and sex as covariate. A list of possible sites is shown in Table 11.

To identify a list of possible sites, FASTQ files (sequencing reads) were checked with FASTQC, trimmed with TrimGalore and aligned to the GRCh38 human reference genome using bwa-meth. The number of methylated and unmethylated counts at each CpG position was calculated using MethylDackel. Beta values were then calculated as the number of methylated reads divided by the total count of reads for each CpG site. For the purpose of downstream modelling, beta values were converted with logarithmic-like transformation to improve their distribution (resulting in M values).

The samples in the M value matrix were split into a 70% training set and 30% independent test set. Pearson correlation was computed in the training set. All CpGs with an absolute Pearson correlation <0.3 were removed from the analysis.

The dimensionality of the remaining CpGs was reduced by applying Singular value decomposition (SVD). This step allowed for the maximization the amount of information within those CpGs but represent them in a much smaller number of features.

A regression model that has a regularisation term was trained to improve its performance and select only relevant features. The model regressed the principal components against chronological age as the outcome variable. To generate predictions on the independent test data, the M values of CpGs were taken from the independent test. These have not yet been transformed by SVD. These M values were projected into the same principal component space as the training data to allow for making predictions on the new data.

The resulting predictions on the independent test data are calculated from the weights given to each of the principal components in the regularized regression model and the scores of the principal components of the independent test data.

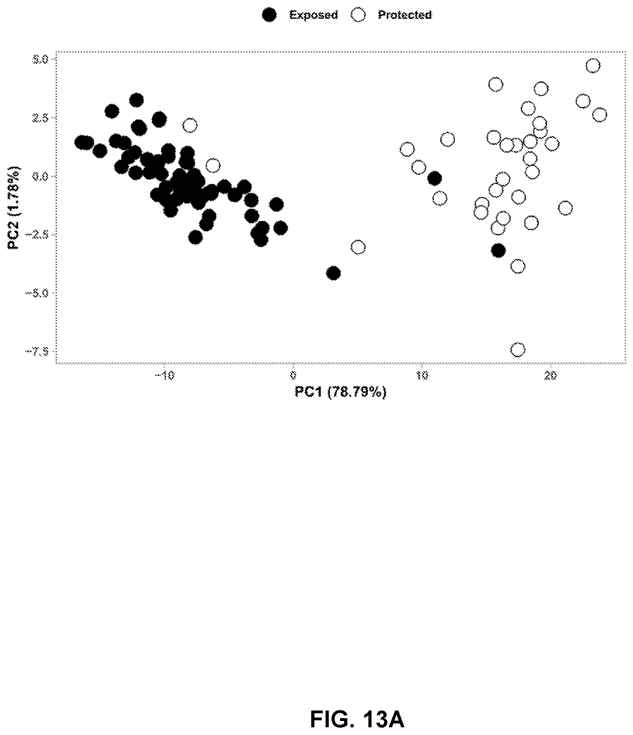

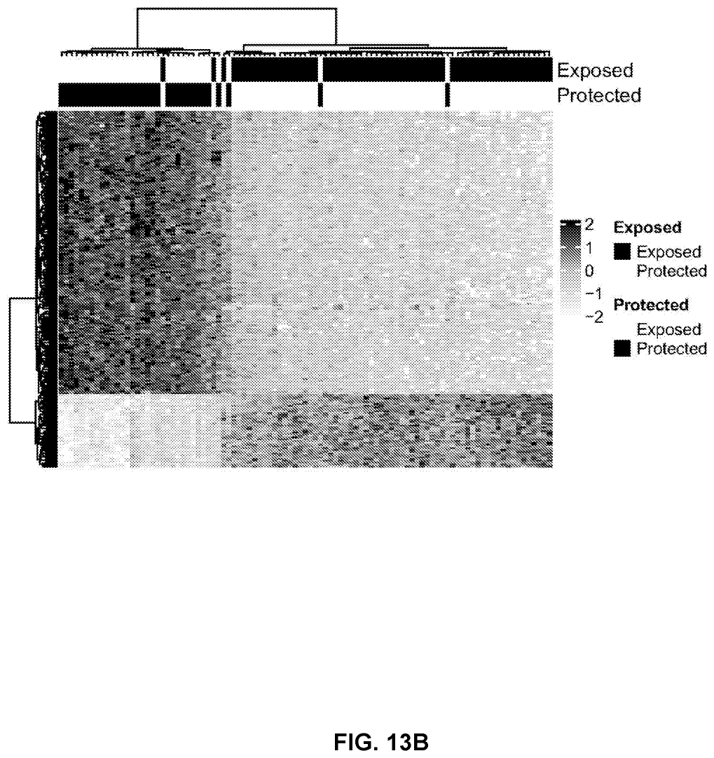

FIGS. 13A-13B show that the DNA methylation patterns change with the UV exposure in epidermal sample collected non-invasively. DNA methylation of the top 200 CpG sites identified through linear regression for differences in UV exposure. The CpG sites were derived from sequencing data from an Illumina NovaSeq instrument. Principal component analysis (FIG. 13A) shows a separation between UV-protected and UV-exposed samples based on methylation differences. Heatmap analysis (FIG. 13B) shows a separation between UV-protected and UV-exposed samples based on methylation differences. X-axis represents different samples. Y-axis represents different methylation sites. The black bars for each “exposed” and “protected” shows the distribution of epigenotypes observed in the 200 sites analyzed in the heatmap.

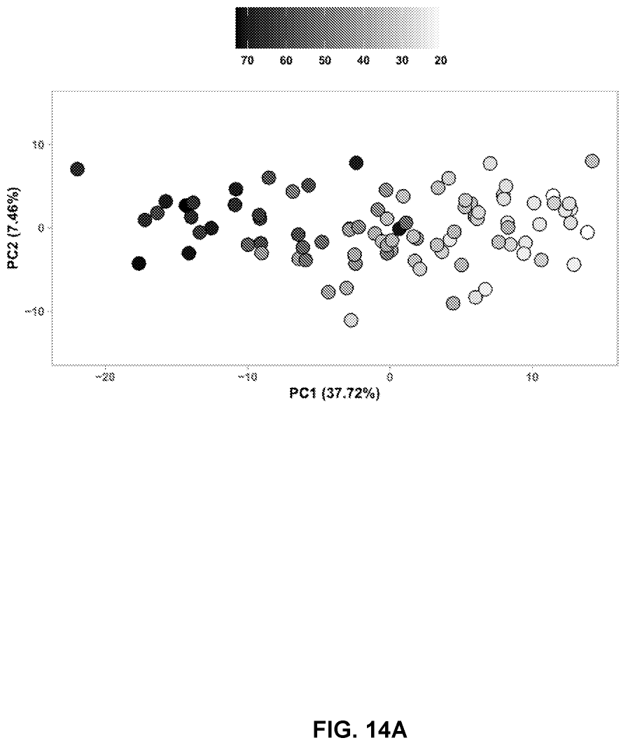

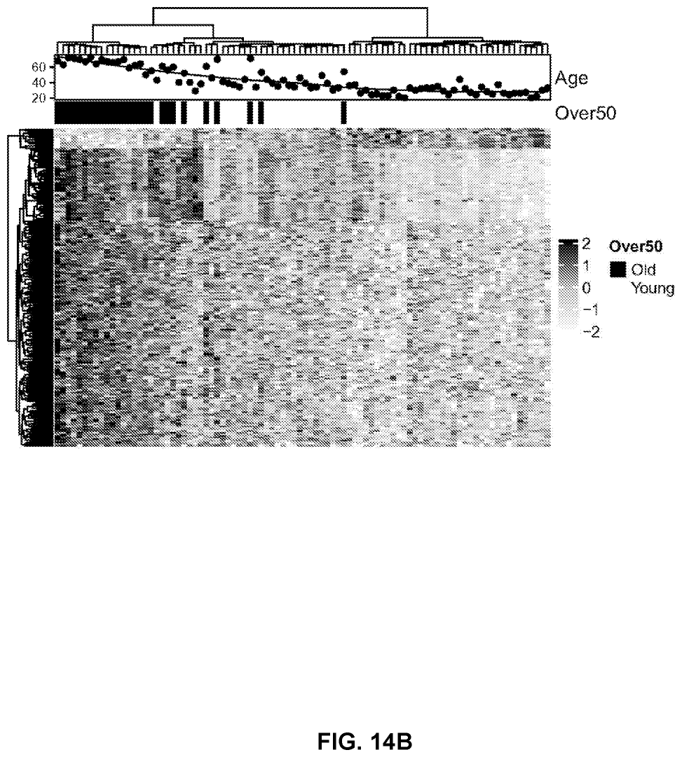

FIGS. 14A-14B show that the DNA methylation patterns change with ages of the subjects in epidermal sample collected non-invasively. DNA methylation of the top 200 CpG sites identified through linear regression for differences in age. The CpG sites were derived from sequencing data from an Illumina NovaSeq instrument. Principal component analysis (FIG. 14A) shows variations in age based on methylation differences. Heatmap analysis (FIG. 14B) shows variations in age based on methylation differences. X-axis represents different samples. Y-axis represents different methylation sites. The black bars for “over 50” shows the distribution of epigenotypes observed in the 200 sites in individuals aged 50 years or more analyzed in the heatmap.

### Example 6: Designing Personalized Epidermal Therapeutic Regimen

Provided herein are methods for designing personalized epidermal therapeutic regimen to improve or alleviate epidermal properties.

The skin patch allows collection of multi-omics data, including genomic (variant analysis), epigenomic (DNA methylation) and metagenomic data. Variant biomarkers provide a deterministic view on the individual genome i.e., sensitivity to cosmetic ingredients, predisposition to selected skin conditions or rate of skin regeneration. On the other hand, the epigenomic and metagenomic data offers a dynamic view on the individual's interaction with the environment i.e., biomarkers associated with ageing depending on environmental metadata or if an ingredient is compatible on the individual's skin. By combining the two types of biomarkers and feeding the data into the machine learning algorithm, one can predict rates of accelerated ageing, sensitivity to ingredients of the individual and predict therapeutic regimens from a curated database.

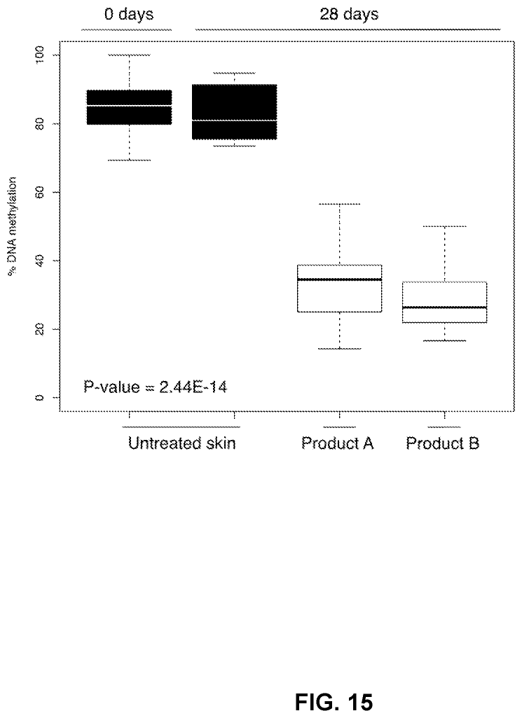

FIG. 15 shows the therapeutic regimen designed to improve epidermal properties. Epidermal samples were collected non-invasively by applying eight adhesive skin tapes on the same spot at the same locations on the forehead: untreated at T0 (day 0 of experiment), untreated at T28 (day 28 of experiment) and treated with either Product A or Product B at T28. Product A or Product B may comprise any therapeutic regimens described elsewhere in this disclosure. DNA extraction was performed as described in EXAMPLE 3. Approximatively 40 ng of the extracted DNA was processed as described in EXAMPLE 4. All samples were sequenced on an Illumina NovaSeq instrument. The sites that show significant changes with product application were identified by running an ANOVA between the four groups, using DNA methylation as the outcome, group as independent variable and age as a covariate. A list of possible sites is shown in Table 11.

Large methylation changes were observed in untreated skin relative to treated skins after 28 days of topical application. UV-damage rejuvenation effect was observed in the treated samples compared to untreated samples. This result is consistent with the epigenotype observed in UV exposed and UV protected samples described in FIG. 12 or EXAMPLE 5.

### Example 7: Correlation of Methylated Sites/Microorganisms and Epidermal Properties

Provided herein are methods for correlating methylation sites or metagenomes and epidermal properties.

The algorithm and method of EXAMPLE 5 are used to identify a list of genes that exhibit specific methylation pattern or level associated with a specific epidermal property. The algorithm and method of EXAMPLE 5 can also be used to identify a list of microorganisms associated with a specific epidermal property.

### Embodiments

1. A method for determining an epidermal property of a subject, comprising:
   (a) providing an epidermal sample of said subject that has been obtained non-invasively from said subject;
   (b) processing at least a portion of nucleic acids derived from said epidermal sample of said subject using at least an enzyme which is capable of modifying a base of a nucleotide of said at least said portion of said nucleic acids thereby producing modified nucleic acids; and
   (c) determining said epidermal property of said subject based at least partially on said modified nucleic acids.
2. The method of embodiment 1, wherein said modifying in (b) comprises converting said base into a different base.
3. The method of embodiment 1 or embodiment 2, wherein said enzyme comprises an enzyme of a Ten-Eleven Translocation (TET) family, an enzyme of a uridine diphosphate (UDP)-glucose: deoxyribonucleic acid (DNA) beta-D-glucosyltransferase family, or an enzyme of a deaminase family.
4. The method of embodiment 3, wherein said enzyme comprises said enzyme of said TET family.
5. The method of embodiment 4, wherein said enzyme of said TET family comprises TET1, TET2, TET3, or a functional equivalent derivative thereof.
6. The method of embodiment 5, wherein said enzyme of said TET family comprises said TET2 or a functional equivalent derivative thereof.
7 The method of embodiment 6, wherein said enzyme comprises said enzyme of said UDP-glucose:DNA beta-D-glucosyltransferase family.
8. The method of embodiment 7, wherein said enzyme of said UDP-glucose:DNA beta-D-glucosyltransferase family is a UDP-glucose:DNA beta-D-glucosyltransferase from E. coli.
9 The method of embodiment 8, wherein said enzyme comprises said enzyme of said deaminase family.
10. The method of embodiment 9, wherein said enzyme of said deaminase family comprises Apolipoprotein B mRNA Editing Catalytic Polypeptide-like 1 (APOBEC1), Apolipoprotein B mRNA Editing Catalytic Polypeptide-like 3A (APOBEC3A), Apolipoprotein B mRNA Editing Catalytic Polypeptide-like 3B (APOBEC3B), Apolipoprotein B mRNA Editing Catalytic Polypeptide-like 3C (APOBEC3C), Apolipoprotein B mRNA Editing Catalytic Polypeptide-like 3D (APOBEC3D), Apolipoprotein B mRNA Editing Catalytic Polypeptide-like 3E (APOBEC3E), Apolipoprotein B mRNA Editing Catalytic Polypeptide-like 3F (APOBEC3F), Apolipoprotein B mRNA Editing Catalytic Polypeptide-like 3H (APOBEC3H), Apolipoprotein B mRNA Editing Catalytic Polypeptide-like 3G (APOBEC3G), Activation-induced cytidine deaminase (AICDA), Cytidine deaminase (CDA), deoxycytidylate monophosphate (dCMP) deaminase (DCTD), or a functional equivalent derivative thereof.
11. The method of embodiment 10, wherein said enzyme of said deaminase family comprises said APOBEC3A or a functional equivalent derivative thereof.
12. The method of embodiment 11, wherein (b) comprises processing said at least said portion of nucleic acids derived from said epidermal sample of said subject using TET2 or a functional equivalent derivative thereof, UDP-glucose:DNA beta-D-glucosyltransferase from E. coli or a functional equivalent derivative thereof, and APOBEC3A or a functional equivalent derivative thereof.
13. The method of any one of embodiments 1-12, further comprising assaying at least a portion of said modified nucleic acids to identify a genome and an epigenome of said epidermal sample, and wherein (c) further comprises determining said epidermal property of said subject based at least partially on said genome and said epigenome of said epidermal sample.
14. The method of embodiment 13, wherein said epigenome comprises a methylome.
15. The method of any one of embodiments 1-14, further comprising obtaining said epidermal sample non-invasively from said subject.
16. The method of embodiment 15, wherein obtaining said epidermal sample non-invasively from said subject comprises applying an adhesive article to a skin area of said subject.
17. The method of embodiment 16, wherein said adhesive article comprises a skin patch.
18. The method of any one of embodiments 1-17, wherein said epidermal sample comprises cells from stratum corneum of said subject.
19. The method of embodiment 18, wherein said cells comprise primary cells.
20. The method of embodiment 19, wherein said primary cells comprise skin cells.
21. The method of embodiment 20, wherein said skin cells comprise keratinocytes.
22. The method of embodiment 21, wherein said keratinocytes comprise corneocytes.
23. The method of any one of embodiments 1-22, further comprising determining a methylation level of at least a portion of said modified nucleic acids, and wherein (c) further comprises determining said epidermal property of said subject based at least partially on said methylation level.
24. The method of embodiment 23, further comprising determining an epidermal property of a reference subject at least in part by determining a reference methylation level of at least a portion of nucleic acids derived from an epidermal sample of said reference subject, and wherein (c) further comprises determining said epidermal property of said subject based at least partially on said reference methylation level.
25. The method of embodiment 24, further comprising comparing said methylation level with said reference methylation level, and wherein (c) further comprises determining said epidermal property of said subject based at least partially on said comparing.
26. The method of embodiment 25, further comprising determining a hypermethylation or hypomethylation of said methylation level relative to said reference methylation level, and wherein (c) further comprises determining said epidermal property of said subject based at least partially on said hypermethylation or hypomethylation of said methylation level.
27. The method of embodiment 25 or embodiment 26, wherein a difference between said methylation level and said reference methylation level is indicative of a difference in said epidermal property of said subject and said epidermal property of said reference subject.
28. The method of any one of embodiments 1-27, further comprising, prior to (b), deriving said nucleic acids from said epidermal sample.
29. The method of embodiment 28, wherein said deriving comprises extracting said nucleic acids from said epidermal sample.
30. The method of embodiment 29, wherein deriving said nucleic acids from said epidermal sample further comprises treating at least a portion of said extracted nucleic acids.
31. The method of embodiment 30, wherein said treating comprises performing an amplification reaction, a reverse transcription reaction, a primer extension reaction, a ligation reaction, or a combination thereof.
32. The method of any one of embodiments 1-31, wherein said nucleic acids derived from said epidermal sample comprise deoxyribonucleic acid (DNA), ribonucleic acid (RNA), or a combination or variant thereof.
33. The method of any one of embodiments 1-32, wherein said nucleic acids derived from said epidermal sample comprise coding sequences or non-coding sequences.
34. The method of embodiment 33, wherein said nucleic acids derived from said epidermal sample comprise said coding sequences.
35. The method of embodiment 33 or embodiment 34, wherein said nucleic acids derived from said epidermal sample comprise said non-coding sequences.
36. The method of any one of embodiments 33-35, wherein said nucleic acids derived from said epidermal sample comprise said coding sequences and said non-coding sequences.
37. The method of any one of embodiments 1-36, further comprising sequencing at least a portion of said modified nucleic acids to produce sequencing reads, and wherein (c) further comprises determining said epidermal property of said subject based at least partially on said sequencing reads.
38. The method of embodiment 37, wherein said sequencing comprises whole-genome sequencing.
39. The method of embodiment 37 or embodiment 38, wherein said sequencing comprises whole-genome methylation sequencing.
40. The method of any one of embodiments 37-39, wherein said sequencing comprises next-generation sequencing.
41. The method of any one of embodiments 37-40, wherein said sequencing comprises chain termination sequencing, high-throughput sequencing, mass spectrophotometry sequencing, massively parallel signature sequencing, Maxam-Gilbert sequencing, nanopore sequencing, primer walking, pyrosequencing, Sanger sequencing, semiconductor sequencing, sequencing-by-hybridization, sequencing-by-ligation, sequencing-by-synthesis, single-molecule sequencing, or shotgun sequencing.
42. The method of any one of embodiments 37-41, wherein said sequencing comprises sequencing-by-synthesis.
43. The method of any one of embodiments 1-42, wherein said epidermal property comprises intrinsic epidermal age, extrinsic epidermal age, epidermal firmness and elasticity, moisture, regeneration capacity, antioxidant protection capacity, inflammatory control, extent of epidermal infection, extent of epidermal disease condition, extent of epidermal pigmentation, extent of ultraviolet (UV) exposure, extent of UV damage, or a combination thereof.
44. The method of any one of embodiments 1-43, wherein said epidermal property comprises a presence or an absence of a skin disease, disorder, or condition.
45. The method of embodiment 44, wherein said skin disease, disorder, or condition is selected from the group consisting of a bruise, a sebaceous cyst, a skin tag, actinic keratoses, allergic contact dermatitis, allergic purpura, an allergic reaction, an infection, basal cell carcinoma, dermatitis, Dermatitis herpetiformis, erythema multiforme, erythema nodosum, hidradenitis suppurative, hives, irritant contact dermatitis, Kaposi's sarcoma, keloid, lipoma, lupus erythematosus, melanoma, Pityriasis rosea, psoriasis, seborrhea, squamous cell carcinoma, xanthelasma, or a combination thereof.
46. The method of embodiment 44 or embodiment 45, further comprising (d) administering a therapeutic regimen to the subject based on said presence of said skin disease, disorder, or condition.
47. The method of embodiment 46, wherein said therapeutic regimen alleviates or improves said epidermal property.
48. The method of embodiment 46 or embodiment 47, wherein said therapeutic regimen comprises an inorganic compound, an organic compound, a drug, a peptide, a protein, a nucleic acid, or a combination thereof.
49. The method of any one of embodiments 46-48, wherein said administering comprises topical application of said therapeutic regimen.
50. The method of embodiment 49, wherein said topical application comprises a hydration mask.
51. The method of any one of embodiments 46-50, wherein said administering comprises an injection of said therapeutic regimen.
52. The method of embodiment 51, wherein said injection comprises onabotulinumtoxinA (Botox®), deoxycholic acid, calcium hydroxylapatite, hyaluronic acid, poly-1-lactic acid, polymethylmethacrylate, bovine collagen filler, or a combination thereof.
53. The method of any one of embodiments 46-52, wherein said administering comprises application of a chemical peel.
54. The method of embodiment 53, wherein chemical peel comprises a chemical solution that causes skin exfoliation and peeling.
55. The method of any one of embodiments 46-54, wherein said therapeutic regimen comprises surgery.
56. The method of embodiment 55, wherein said surgery comprises cryosurgery, laser surgery, dermabrasion, liposuction, microlipoinjection, microneedling, micropigmentation, microwave thermolysis, neck lift, laser rejuvenation, body contouring, skin tightening, vein treatment, or a combination thereof.
57. The method of any one of embodiments 46-56, further comprising providing a second epidermal sample from a skin area of said subject from which said epidermal sample has been obtained.
58. The method of embodiment 57, further comprising performing one or more additional iterations of (b) and (c) to monitor said epidermal property of said subject responsive to said administering.
59. A method for determining an epidermal property of a subject, comprising:
   (a) providing an epidermal sample of said subject that has been obtained non-invasively from said subject;
   (b) sequencing at least a portion of nucleic acids derived from said epidermal sample of said subject to determine a transcriptome and a genome of said epidermal sample; and
   (c) determining said epidermal property of said subject based at least partially on said transcriptome and said genome of said epidermal sample.
60. The method of embodiment 59, wherein said genome comprises an epigenome.
61 The method of embodiment 60, wherein said epigenome comprises a methylome.
62 The method of any one of embodiments 59-61, further comprising obtaining said epidermal sample non-invasively from said subject.
63. The method of embodiment 62, wherein obtaining said epidermal sample non-invasively from said subject comprises applying an adhesive article to a skin area of said subject.
64. The method of embodiment 63, wherein said adhesive article comprises a skin patch.
65. The method of any one of embodiments 59-64, wherein (b) further comprises determining a metagenome of said epidermal sample, and wherein (c) further comprises determining said epidermal property of said subject based at least partially on said metagenome of said epidermal sample.
66. The method of embodiment 65, wherein said metagenome comprises a bacterium, a virus, an archaeon, a fungus, a protozoon, or a combination thereof.
67. The method of embodiment 66, wherein said metagenome comprises at least two of said bacterium, said virus, said archaea, said fungus, and said protozoon.
68. The method of embodiment 67, wherein said metagenome comprises at least three of said bacterium, said virus, said archaea, said fungus, or said protozoon.
69. The method of embodiment 68, wherein said metagenome comprises said bacterium, said virus, said archaea, said fungus, and said protozoon.
70. The method of any one of embodiments 59-69, wherein said epidermal sample comprises cells from stratum corneum of said subject.
71. The method of embodiment 70, wherein said cells comprise primary cells.
72. The method of embodiment 71, wherein said primary cells comprise skin cells.
73. The method of embodiment 72, wherein said skin cells comprise keratinocytes.
74. The method of embodiment 73, wherein said keratinocytes comprise corneocytes.
75. The method of any one of embodiments 59-74, further comprising, prior to (b), deriving said nucleic acids from said epidermal sample.
76. The method of embodiment 75, wherein said deriving comprises extracting said nucleic acids from said epidermal sample.
77. The method of embodiment 76, wherein said comprises treating at least a portion of said extracted nucleic acids.
78. The method of embodiment 77, wherein said treating comprises performing an amplification reaction, a reverse transcription reaction, a primer extension reaction, a ligation reaction, or a combination thereof.
79. The method of any one of embodiments 59-78, wherein said extracted nucleic acids or said nucleic acids derived from said epidermal sample comprise deoxyribonucleic acid (DNA), ribonucleic acid (RNA), or a combination or variant thereof.
80. The method of any one of embodiments 59-79, wherein said extracted nucleic acids or said nucleic acids derived from said epidermal sample comprise coding sequences or non-coding sequences.
81. The method of embodiment 80, wherein said extracted nucleic acids or said nucleic acids derived from said epidermal sample comprise said coding sequences.
82. The method of embodiment 80 or embodiment 81, wherein said extracted nucleic acids or said nucleic acids derived from said epidermal sample comprise said non-coding sequences.
83. The method of any one of embodiments 80-82, wherein said extracted nucleic acids or said nucleic acids derived from said epidermal sample comprise said coding sequences and said non-coding sequences.
84. The method of any one of embodiments 59-83, wherein said sequencing comprises whole-genome sequencing.
85. The method of any one of embodiments 59-84, wherein said sequencing comprises whole-genome methylation sequencing.
86. The method of any one of embodiments 59-85, wherein said sequencing comprises next-generation sequencing.
87. The method of any one of embodiments 59-86, wherein said sequencing comprises chain termination sequencing, high-throughput sequencing, mass spectrophotometry sequencing, massively parallel signature sequencing, Maxam-Gilbert sequencing, nanopore sequencing, primer walking, pyrosequencing, Sanger sequencing, semiconductor sequencing, sequencing-by-hybridization, sequencing-by-ligation, sequencing-by-synthesis, single-molecule sequencing, or shotgun sequencing.
88. The method of any one of embodiments 59-87, wherein said sequencing comprises sequencing-by-synthesis.
89. The method of any one of embodiments 59-88, wherein said epidermal property comprises intrinsic epidermal age, extrinsic epidermal age, epidermal firmness and elasticity, moisture, regeneration capacity, antioxidant protection capacity, inflammatory control, extent of epidermal infection, extent of epidermal disease condition, extent of epidermal pigmentation, extent of ultraviolet (UV) exposure, extent of UV damage, or a combination thereof.
90. The method of any one of embodiments 59-89, wherein said epidermal property comprises a presence or an absence of a skin disease, disorder, or condition.
91. The method of embodiment 90, wherein said skin disease, disorder, or condition is selected from the group consisting of a bruise, a sebaceous cyst, a skin tag, actinic keratoses, allergic contact dermatitis, allergic purpura, an allergic reaction, an infection, basal cell carcinoma, dermatitis, Dermatitis herpetiformis, erythema multiforme, erythema nodosum, hidradenitis suppurative, hives, irritant contact dermatitis, Kaposi's sarcoma, keloid, lipoma, lupus erythematosus, melanoma, Pityriasis rosea, psoriasis, seborrhea, squamous cell carcinoma, xanthelasma, or a combination thereof.
92. The method of embodiment 90 or embodiment 91, further comprising (d) administering a therapeutic regimen to said subject based on said presence of said skin disease, disorder, or condition.
93. The method of embodiment 92, wherein said therapeutic regimen alleviates or improves said epidermal property.
94 The method of embodiment 92 or embodiment 93, wherein said therapeutic regimen comprises an inorganic compound, an organic compound, a drug, a peptide, a protein, a nucleic acid, or a combination thereof.
95. The method of any one of embodiments 92-94, wherein said administering comprises topical application of said therapeutic regimen.
96. The method of embodiment 95, wherein said topical application comprises a hydration mask.
97. The method of any one of embodiments 92-96, wherein said administering comprises an injection of said therapeutic regimen.
98. The method of embodiment 97, wherein said injection comprises onabotulinumtoxinA (Botox®), deoxycholic acid, calcium hydroxylapatite, hyaluronic acid, poly-1-lactic acid, polymethylmethacrylate, bovine collagen filler, or a combination thereof.
99. The method of any one of embodiments 92-98, wherein said administering comprises application of a chemical peel.
100. The method of embodiment 99, wherein chemical peel comprises a chemical solution that causes skin exfoliation and peeling.
101. The method of any one of embodiments 92-100, wherein said therapeutic regimen comprises surgery.
102. The method of embodiment 101, wherein said surgery comprises cryosurgery, laser surgery, dermabrasion, liposuction, microlipoinjection, microneedling, micropigmentation, microwave thermolysis, neck lift, laser rejuvenation, body contouring, skin tightening, vein treatment, or a combination thereof.
103. The method of any one of embodiments 92-102, further comprising providing a second epidermal sample from a skin area of said subject from which said epidermal sample has been obtained.
104. The method of embodiment 103, further comprising performing one or more additional iterations of (b) and (c) to monitor said epidermal property of said subject responsive to said administering.
105. A method for determining an epidermal property of a subject, comprising:
   (a) providing an epidermal sample of said subject;
   (b) assaying nucleic acid molecules derived from said epidermal sample to identify:
      (i) a first set of nucleic acid sequences from said subject; and
      (ii) a second set of nucleic acid sequences from a source different from said subject;
   (c) using a trained algorithm to process said first set of nucleic acid sequences and said second set of nucleic acid sequences to determine a likelihood of said epidermal sample being positive or negative for said epidermal property.
106. The method of embodiment 105, wherein said source different from said subject comprises a species different from said subject.
107. The method of embodiment 106, wherein said species comprises a bacterium, a virus, an archaeon, a fungus, a protozoon, or a combination thereof.
108. The method of any one of embodiments 105-107, wherein said epidermal sample is obtained in an invasive, non-invasive, or semi-invasive manner.
109. The method of any one of embodiments 105-108, wherein said epidermal sample is obtained in a non-invasive manner.
110. The method of embodiment 109, wherein said non-invasive manner comprises applying an adhesive article to a skin area of said subject.
111. The method of embodiment 110, wherein said adhesive article comprises a skin patch.
112. The method of any one of embodiments 105-111, wherein said epidermal sample is obtained in an invasive manner.
113. The method of embodiment 112, wherein said invasive manner comprises a biopsy.
114. The method of embodiment 113, wherein said biopsy comprises a punch biopsy.
115. The method of any one of embodiments 105-114, wherein said first set of nucleic acid sequences comprises a transcriptome, a genome, or a combination thereof.
116. The method of embodiment 115, wherein said first set of nucleic acid sequences comprises said genome.
117. The method of embodiment 116, wherein said genome comprises an epigenome.
118. The method of embodiment 117, wherein said epigenome comprises a methylome.
119. The method of any one of embodiments 105-118, wherein said second set of nucleic acid sequences of said nucleic acids comprises a metagenome.
120. The method of any one of embodiments 105-119, wherein said epidermal sample comprises cells from stratum corneum of said subject.
121. The method of embodiment 120, wherein said cells comprise primary cells.
122. The method of embodiment 121, wherein said primary cells comprise skin cells.
123. The method of embodiment 122, wherein said skin cells comprise keratinocytes.
124. The method of embodiment 123, wherein said keratinocytes comprise corneocytes.
125. The method of any one of embodiments 105-124, further comprising, prior to (b), extracting said nucleic acid molecules from said epidermal sample.
126. The method of any one of embodiments 105-125, further comprising, prior to (b), processing said nucleic acid molecules extracted from said epidermal sample to form said nucleic acid molecules derived from said epidermal sample.
127. The method of embodiment 126, wherein said processing comprises an amplification reaction, a reverse transcription reaction, or a combination thereof.
128. The method of embodiment 127, wherein said processing comprises said amplification reaction.
129. The method of embodiment 127 or embodiment 128, wherein said processing comprises said reverse transcription reaction.
130. The method of any one of embodiments 105-129, wherein said nucleic acid molecules derived from said epidermal sample comprise deoxyribonucleic acid (DNA), ribonucleic acid (RNA), or a combination or variant thereof.
131. The method of embodiment 130, wherein said nucleic acid molecules derived from said epidermal sample comprise DNA.
132. The method of embodiment 130 or embodiment 131, wherein said nucleic acid molecules derived from said epidermal sample comprise RNA.
133. The method of any one of embodiments 130-132, wherein said nucleic acid molecules derived from said epidermal sample comprise DNA and RNA.
134. The method of any one of embodiments 105-133, wherein said first set of nucleic acid sequences comprises coding sequences and/or non-coding sequences.
135. The method of embodiment 134, wherein said first set of nucleic acid sequences comprises said coding sequences.
136. The method of embodiment 134 or embodiment 135, wherein said first set of nucleic acid sequences comprises said non-coding sequences.
137. The method of any one of embodiments 134-136, wherein said first set of nucleic acid sequences comprises said coding sequences and said non-coding sequences.
138. The method of any one of embodiments 105-137, wherein said assaying of (b) comprises sequencing.
139. The method of any one of embodiments 105-138, wherein said assaying of (b) comprises whole-genome sequencing.
140. The method of any one of embodiments 105-139, wherein said assaying of (b) comprises whole-genome methylation sequencing.
141. The method of any one of embodiments 105-140, wherein said assaying of (b) comprises next-generation sequencing.
142. The method of any one of embodiments 105-141, wherein said assaying of (b) comprises chain termination sequencing, high-throughput sequencing, mass spectrophotometry sequencing, massively parallel signature sequencing, Maxam-Gilbert sequencing, nanopore sequencing, primer walking, pyrosequencing, Sanger sequencing, semiconductor sequencing, sequencing-by-hybridization, sequencing-by-ligation, sequencing-by-synthesis, single-molecule sequencing, or shotgun sequencing.
143. The method of any one of embodiments 105-142, wherein said assaying of (b) comprises sequencing-by-synthesis.
144. The method of any one of embodiments 105-143, wherein said epidermal property comprises intrinsic epidermal age, extrinsic epidermal age, epidermal firmness and elasticity, moisture, regeneration capacity, antioxidant protection capacity, inflammatory control, extent of epidermal infection, extent of epidermal disease condition, extent of epidermal pigmentation, extent of UV exposure, extent of UV damage, or a combination thereof.
145. The method of any one of embodiments 105-144, wherein (c) further comprises determining a likelihood of occurrence or non-occurrence of said epidermal property in said subject using said trained algorithm.
146. The method of embodiment 145, wherein said trained algorithm comprises supervised learning, non-supervised learning, semi-supervised learning or any combination thereof.
147. The method of any one of embodiments 105-146, further comprising (d) administering a therapeutic regimen to said subject.
148. The method of embodiment 147, wherein said therapeutic regimen alleviates or improves said epidermal property.
149. The method of any one of embodiments 105-148, further comprising providing an additional epidermal sample of said subject.
150. The method of embodiment 149, wherein said additional epidermal sample is obtained from a skin area of said subject from which said epidermal sample is obtained.
151. The method of embodiment 149 or embodiment 150, further comprising repeating steps (b)-(c) one or more times to determine the epidermal property of said subject subsequent to said subject being administered to said therapeutic regimen.
152. A method for determining an epidermal property of a subject, comprising:
   (a) obtaining an epidermal sample non-invasively from said subject;
   (b) processing at least a portion of nucleic acids derived from said epidermal sample; and
   (c) determining said epidermal property of said subject based at least partially on said portion of said nucleic acids processed in (b), wherein $ said portion of said nucleic acids comprises a sequence of a genome of said subject defined in Table 11 or a functional equivalent thereof.
153. The method of embodiment 152, wherein (b) comprises processing said portion of nucleic acids derived from said epidermal sample using at least an enzyme which is capable of modifying a base of a nucleotide of said portion of said nucleic acids 154. The method of embodiment 153, wherein said modifying said base comprises converting said base into a different base.
155. The method of embodiment 153 or embodiment 154, wherein said enzyme comprises an enzyme of a Ten-Eleven Translocation (TET) family, an enzyme of a UDP-glucose:DNA beta-D-glucosyltransferase family, or an enzyme of a deaminase family.
156. The method of embodiment 155, wherein said enzyme of said TET family comprises TET1, TET2, TET3, or a functional equivalent derivative thereof.
157. The method of embodiment 156, wherein said enzyme of said TET family comprises said TET2 or a functional equivalent derivative thereof.
158. The method of any one of embodiments 155-157, wherein said enzyme of said UDP-glucose:DNA beta-D-glucosyltransferase family is a UDP-glucose:DNA beta-D-glucosyltransferase from E. coli.
159. The method of any one of embodiments 155-158, wherein said enzyme of said deaminase family comprises Apolipoprotein B mRNA Editing Catalytic Polypeptide-like 1 (APOBEC1), Apolipoprotein B mRNA Editing Catalytic Polypeptide-like 3A (APOBEC3A), Apolipoprotein B mRNA Editing Catalytic Polypeptide-like 3B (APOBEC3B), Apolipoprotein B mRNA Editing Catalytic Polypeptide-like 3C (APOBEC3C), Apolipoprotein B mRNA Editing Catalytic Polypeptide-like 3D (APOBEC3D), Apolipoprotein B mRNA Editing Catalytic Polypeptide-like 3E (APOBEC3E), Apolipoprotein B mRNA Editing Catalytic Polypeptide-like 3F (APOBEC3F), Apolipoprotein B mRNA Editing Catalytic Polypeptide-like 3H (APOBEC3H), Apolipoprotein B mRNA Editing Catalytic Polypeptide-like 3G (APOBEC3G), Activation-induced cytidine deaminase (AICDA), Cytidine deaminase (CDA), deoxycytidylate monophosphate (dCMP) deaminase (DCTD), or a functional equivalent derivative thereof.
160. The method of any one of embodiments 155-159, wherein said enzyme of said deaminase family comprises said APOBEC3A or a functional equivalent derivative thereof.
161. The method of any one of embodiments 155-160, wherein (b) comprises processing at least said portion of nucleic acids derived from said epidermal sample of said subject using said TET2 or any functional equivalent derivative thereof, said UDP-glucose:DNA beta-D-glucosyltransferase from E. coli or any functional equivalent derivative thereof, and said APOBEC3A or any functional equivalent derivative thereof.
162. The method of any one of embodiments 152-161, further comprising, prior to (c), assaying said at least said portion of said nucleic acids to identify a genome and an epigenome of said epidermal sample.
163. The method of embodiment 162, wherein said epigenome comprises a methylome.
164. The method of any one of embodiments 152-163, wherein obtaining said epidermal sample non-invasively from said subject comprises applying an adhesive article to a skin area of said subject.
165. The method of embodiment 164, wherein said adhesive article comprises a skin patch.
166. The method of any one of embodiments 152-165, wherein said epidermal sample comprises cells from stratum corneum of said subject.
167. The method of any one of embodiments 152-166, wherein (c) comprises determining a methylation level of said portion of said nucleic acid processed in (b).
168. The method of embodiment 167, further comprising determining an epidermal property of a reference subject, wherein said determining said epidermal property of said reference subject comprises determining a methylation level of said portion of said nucleic acid derived from an epidermal sample of said reference subject.
169. The method of embodiment 168, further comprising comparing said methylation level of said portion of said nucleic acid derived from said epidermal sample of said subject with said methylation level of said portion of said nucleic acid derived from said epidermal sample of said reference subject.
170. The method of embodiment 169, wherein a difference in said methylation level of said portion of said nucleic acid derived from said epidermal sample of said subject and said methylation level of said portion of said nucleic acid derived from said epidermal sample of said reference subject represents a difference in said epidermal property of said subject and said epidermal property of said reference subject.
171. The method of any one of embodiments 152-170, further comprising, prior to (b), extracting said nucleic acids from said epidermal sample.
172. The method of embodiment 171, further comprising, prior to (b), treating said nucleic acids extracted from said epidermal sample to form said nucleic acids derived from said epidermal sample.
173. The method of embodiment 172, wherein said treating comprises an amplification reaction, a reverse transcription reaction, or a combination thereof.
174. The method of any one of embodiments 152-173, wherein said nucleic acids derived from said epidermal sample comprise deoxyribonucleic acid (DNA), ribonucleic acid (RNA), or a combination or variant thereof.
175. The method of any one of embodiments 152-174, wherein said nucleic acids derived from said epidermal sample comprise coding sequences or non-coding sequences.
176. The method of embodiment 175, wherein said nucleic acids derived from said epidermal sample comprise coding sequences.
177. The method of embodiment 175 or embodiment 176, wherein said nucleic acids derived from said epidermal sample comprise non-coding sequences.
178. The method of any one of embodiments 175-177, wherein said nucleic acids derived from said epidermal sample comprise coding sequences and non-coding sequences.
179. The method of any one of embodiments 152-178, further comprising, prior to (c), sequencing said nucleic acids derived from said epidermal sample.
180. The method of embodiment 179, wherein said sequencing comprises whole-genome sequencing.
181. The method of embodiment 179 or embodiment 180, wherein said sequencing comprises whole-genome methylation sequencing.
182. The method of any one of embodiments 179-181, wherein said sequencing comprises next-generation sequencing.
183. The method of any one of embodiments 179-182, wherein said sequencing comprises chain termination sequencing, high-throughput sequencing, mass spectrophotometry sequencing, massively parallel signature sequencing, Maxam-Gilbert sequencing, nanopore sequencing, primer walking, pyrosequencing, Sanger sequencing, semiconductor sequencing, sequencing-by-hybridization, sequencing-by-ligation, sequencing-by-synthesis, single-molecule sequencing, or shotgun sequencing.
184. The method of any one of embodiments 179-183, wherein said sequencing comprises sequencing-by-synthesis.
185. The method of any one of embodiments 152-184, wherein said epidermal property comprises intrinsic epidermal age, extrinsic epidermal age, epidermal firmness and elasticity, moisture, regeneration capacity, antioxidant protection capacity, inflammatory control, extent of epidermal infection, extent of epidermal disease condition, extent of epidermal pigmentation, extent of UV exposure, extent of UV damage, or a combination thereof.
186. The method of any one of embodiments 152-185, further comprising (d) administering a therapeutic regimen to the subject.
187. The method of embodiment 186, wherein said therapeutic regimen alleviates or improves said epidermal property.
188. The method of any one of embodiments 152-187, further comprising obtaining an additional epidermal sample from a skin area of said subject from which said epidermal sample is obtained.
189. The method of embodiment 188, further comprising repeating steps (b)-(c) one or more times to determine the epidermal property of said subject subsequent to said subject being administered to said therapeutic regimen.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for determining an epidermal property of a subject, comprising:
(a) providing an epidermal sample of said subject that has been obtained non-invasively from said subject;
(b) processing at least a portion of nucleic acids derived from said epidermal sample of said subject using at least an enzyme which is capable of modifying a base of a nucleotide of said at least said portion of said nucleic acids thereby producing modified nucleic acids; and
(c) determining said epidermal property of said subject based at least partially on said modified nucleic acids.

2. The method of claim 1, wherein said modifying in (b) comprises converting said base into a different base.

3. The method of claim 1, wherein said enzyme comprises an enzyme of a Ten-Eleven Translocation (TET) family, an enzyme of a uridine diphosphate (UDP)-glucose:deoxyribonucleic acid (DNA) beta-D-glucosyltransferase family, or an enzyme of a deaminase family.

4-189. (canceled)

190. The method of claim 1, wherein (b) comprises processing said at least said portion of nucleic acids derived from said epidermal sample of said subject using TET2 or a functional equivalent derivative thereof, UDP-glucose:DNA beta-D-glucosyltransferase from E. coli or a functional equivalent derivative thereof, and APOBEC3A or a functional equivalent derivative thereof.

191. The method of claim 1, further comprising assaying at least a portion of said modified nucleic acids to identify a genome and an epigenome of said epidermal sample, and wherein (c) further comprises determining said epidermal property of said subject based at least partially on said genome and said epigenome of said epidermal sample.

192. The method of claim 1, further comprising obtaining said epidermal sample non-invasively from said subject.

193. The method of claim 192, further comprising applying an adhesive article comprising a skin tape to a skin area of said subject.

194. The method of claim 1, wherein said epidermal sample comprises cells from at least one of a stratum corneum and a stratum granulosum of said subject.

195. The method of claim 1, further comprising determining a methylation level of at least a portion of said modified nucleic acids, and wherein (c) further comprises determining said epidermal property of said subject based at least partially on said methylation level.

196. The method of claim 195, wherein the determining further comprises determining an epidermal property of a reference subject at least in part by determining a reference methylation level of at least a portion of nucleic acids derived from an epidermal sample of said reference subject, and wherein (c) further comprises determining said epidermal property of said subject based at least partially on said reference methylation level.

197. The method of claim 193, further comprising comparing said methylation level with said reference methylation level, and wherein (c) further comprises determining said epidermal property of said subject based at least partially on said comparing.

198. The method of claim 194, further comprising determining a hypermethylation or hypomethylation of said methylation level relative to said reference methylation level, and wherein (c) further comprises determining said epidermal property of said subject based at least partially on said hypermethylation or hypomethylation of said methylation level.

199. The method of claim 195, wherein a difference between said methylation level and said reference methylation level is indicative of a difference in said epidermal property of said subject and said epidermal property of said reference subject.

200. The method of claim 1, further comprising, prior to (b), deriving said nucleic acids from said epidermal sample.

201. The method of claim 1, wherein said nucleic acids derived from said epidermal sample comprise deoxyribonucleic acid (DNA), ribonucleic acid (RNA), or a combination or variant thereof.

202. The method of claim 1, further comprising sequencing at least a portion of said modified nucleic acids to produce sequencing reads, and wherein (c) further comprises determining said epidermal property of said subject based at least partially on said sequencing reads.

203. The method of claim 202, wherein the sequencing comprises whole-genome sequencing, more preferably whole-genome methylation sequencing, or next-generation sequencing, chain termination sequencing, high-throughput sequencing, mass spectrophotometry sequencing, massively parallel signature sequencing, Maxam-Gilbert sequencing, nanopore sequencing, primer walking, pyrosequencing, Sanger sequencing, semiconductor sequencing, sequencing-by-hybridization, sequencing-by-ligation, sequencing-by-synthesis, single-molecule sequencing, shotgun sequencing or using a solid support comprising a bead.

204. The method of claim 1, wherein said epidermal property comprises intrinsic epidermal age, extrinsic epidermal age, epidermal firmness and elasticity, moisture, regeneration capacity, antioxidant protection capacity, inflammatory control, extent of epidermal infection, extent of epidermal disease condition, extent of epidermal pigmentation, extent of ultraviolet (UV) exposure, extent of UV damage, or a combination thereof.

205. The method of claim 1, wherein said epidermal property comprises a presence or an absence of a skin disease, disorder, or condition.

206. The method of claim 1, further comprising (d) administering a therapeutic regimen to the subject based on said presence of said skin disease, disorder, or condition.
